SEQUENCE LISTING (22 sequences)

SEQ ID NO: 1 - DNA, 411 bp, Mus musculus
atggaatggagctgggtctttctcttcttcctgtcagtaactacaggtgtccactcccaggttcagctgcagcagtctgacgctgagttggtgaaacctggggcttcagtgaagatatcctgcaaggcttctggctacaccttcactgaccatgctattcactgggtgaagcagaagcctgaacagggcctggaatggattggatatatttctcccggaaatgatgttattaagttcaatgagaagttcaagggcaaggccacactgactgcagacaaatcctccagcactgcctacatgcagctcaacagcctgacatctgaggattctgcagtgtatttctgtaagagatcagaggggggggtttttgactactggggccaaggcaccactctcacagtctcctcagag

SEQ ID NO: 2 - DNA, 399 bp, Mus musculus
atggaatcacagactcaggtcttcctctccctgctgctctgggtatctggtacctgtgggaacattatgatgacacagtcgccatcatctctggctgtgtctgcaggagaaaaggtcactatgagctgtaagtccagtcaaagtgttttatacagttcaaatcagaagaacttcttggcctggtaccagcagaaaccaggacagtctcctaaactgctgatctactgggcatccactagggaatctggtgtccctgatcgcttcacaggcagtggatctgggacagattttactcttaccatcagcagtgtacaagctgaagacctggcagtttattactgtcatcaatacctctcctcgtacacgttcggaggggggaccaagctggaaataaaacgg

SEQ ID NO: 3 - protein, 474 aa, artificial sequence
  Chimeric mus musculus/homo sapiens
MEWSWVFLFFLSVTTGVHSQVQLQQSDAELVKPGASVKISCKASGYTFTDHAIHWVKQKPEQGLEWIGYISPGNDVIKFNEKFKGKATLTADKSSSTAYMQLNSLTSEDSAVYFCKRSEGGVFDYWGQGTTLTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPGTEPKSADKTHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 239 aa, Mus musculus
MESQTQVFLSLLLWVSGTCGNIMMTQSPSSLAVSAGEKVTMSCKSSQSVLYSSNQKNFLAWYQQKPGQSPKLLIYWASTRESGVPDRFTGSGSGTDFTLTISSVQAEDLAVYYCHQYLSSYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 447 aa, artificial sequence
  Chimeric mus musculus/homo sapiens
QVQLQQSDAELVKPGASVKISCKASGYTFTDHAIHWVKQKPEQGLEWIGYISPGNDVIKFNEKFKGKATLTADKSSSTAYMQLNSLTSEDSAVYFCKRSEGGVFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 216 aa, artificial sequence
  Chimeric mus musculus/homo sapiens
NIMMTQSPSSLAVSAGEKVTMSCKSSQSVLYSSNQKNFLAWYQQKPGQSPKLLIYWASTRESGVPDRFTGSGSGTDFTLTISSVQAEDLAVYYCHQYLSSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 7 - protein, 116 aa, Mus musculus
QVQLQQSDAELVKPGASVKISCKASGYTFTDHAIHWVKQKPEQGLEWIGYISPGNDVIKFNEKFKGKATLTADKSSSTAYMQLNSLTSEDSAVYFCKRSEGGVFDYWGQGTTLTVS

SEQ ID NO: 8 - protein, 112 aa, Mus musculus
NIMMTQSPSSLAVSAGEKVTMSCKSSQSVLYSSNQKNFLAWYQQKPGQSPKLLIYWASTRESGVPDRFTGSGSGTDFTLTISSVQAEDLAVYYCHQYLSSYTFGGGTKLEIK

SEQ ID NO: 9 - protein, 5 aa, Mus musculus
DHAIH

SEQ ID NO: 10 - protein, 18 aa, Mus musculus
YISPGNDVIKFNEKFKGK

SEQ ID NO: 11 - protein, 10 aa, Mus musculus
KRSEGGVFDY

SEQ ID NO: 12 - protein, 17 aa, Mus musculus
KSSQSVLYSSNQKNFLA

SEQ ID NO: 13 - protein, 7 aa, Mus musculus
WASTRES

SEQ ID NO: 14 - protein, 8 aa, Mus musculus
HQYLSSYT

SEQ ID NO: 15 - protein, 331 aa, Homo sapiens
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 104 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 17 - DNA, 23 bp, Artificial
  PCR primer
gtgaaggtcggtgtgaacggatt

SEQ ID NO: 18 - DNA, 23 bp, Artificial
  PCR primer
ctaagcagttggtggtgcaggat

SEQ ID NO: 19 - DNA, 29 bp, Artificial
  PCR primer
aagctagcatggaatcacagactcaggct

SEQ ID NO: 20 - DNA, 32 bp, Artificial
  PCR primer
aagcggccgcctaacactcatttctgttgaag

SEQ ID NO: 21 - DNA, 30 bp, Artificial
  PCR primer
aagaattcatggaatggagctgggtctttc

SEQ ID NO: 22 - DNA, 29 bp, Artificial
  PCR primer
aaggtacctggaatgggcacatgcagatc

(19) United States
(12) Patent Application Publication
(10) Pub. No.: US 2010/0124555 A1
(43) Pub. Date: May 20, 2010

(54) Compositions and Methods for Treating Pancreatic Tumors

(75) Inventors: LYDIE CRESCENCE, MARSEILLE (FR); LAURENT GAUTHIER, MARSEILLE (FR); DOMINIQUE LOMBARDO, MARSEILLE (FR); ERIC MAS, MARSEILLE (FR); BENJAMIN ROSSI, MARSEILLE (FR)

Correspondence Address: SALIWANCHIK LLOYD & SALIWANCHIK;A PROFESSIONAL ASSOCIATION, PO Box 142950, GAINESVILLE, FL, 32614, US

(73) Assignees: UNIVERSITE DE LA MEDITERRANEE, MARSEILLE CEDEX 07 (FR); INSERM (INSTITUT NATIONAL DE LA SANTE ET DE LA RECHERCHE MEDICALE), PARIS CEDEX 13 (FR); INNATE PHARMA S.A., MARSEILLE (FR)

(21) Appl. No.: 12/612,874

(22) Filed: Nov. 5, 2009

Related U.S. Application Data
(63) Continuation-in-part of application No. PCT/EP2008/057112, filed on Jun. 6, 2008.
(60) Provisional application No. 60/942,777, filed on Jun. 8, 2007.

(51) Int. Cl. A61K 39/395; G01N 33/53; A61P 35/00; C07K 16/00; C12N 5/00

(52) U.S. Cl. 424/174.1; 435/7.21; 435/7.23; 530/389.7; 530/387.3; 530/391.7; 530/391.3; 435/375

(57) ABSTRACT

The present invention relates to a method for producing an antigen-binding compound suitable for use in the treatment of cancer, the antigen-binding compounds and their uses.

### CROSS-REFERENCE TO RELATED APPLICATIONS

This application is a continuation-in-part of PCT International Application No. PCT/EP2008/057112, filed Jun. 6, 2008, which claims the benefit of U.S. Provisional Application Ser. No. 60/942,777, filed Jun. 8, 2007, the disclosures of which are hereby incorporated by reference in their entirety, including all figures, tables and amino acid or nucleic acid sequences.

### FIELD OF THE INVENTION

The invention relates to glycopeptides derived from pancreatic structures, antibodies and applications thereof in diagnostics and therapeutics, methods of obtaining antigen binding compounds, as well as treatment regimens using the antibodies, optionally further in combination with other therapeutic agents.

### BACKGROUND

Cancer of the exocrine pancreas, which accounts for over 20% of digestive tract cancers, is one of the most aggressive forms of cancer. In France, for example, 4,000 new cases are diagnosed each year. Further, its frequency is rising markedly in many regions of the world. Survival rates do not exceed 20% at 1 year and 3% at 5 years, and mean survival is 3 to 4 months after diagnosis. This low survival rate stems from numerous causes, including the fact that the deep anatomic location of the tumor, the absence of sensitive and specific early biological markers, and its asymptomatic nature result in a diagnosis that virtually always occurs late. In addition, pancreatic cancer progresses very rapidly, mainly through the formation of peritoneal and hepatic metastases. Currently there are insufficient therapeutic options for the treatment of pancreatic cancer.

Two distinct approaches have been explored for the treatment of pancreatic cancers: chemoradiation and immunotherapy. Chemoradiation clinical trials include gemcitabine added to a standard chemoradiation regimen; this has been shown to slightly improve the overall survival of patients. The addition of erlotinib to gemcitabine only provides a modest improvement. In general, chemoradiation treatments, although showing some positive results, still represent a poor therapeutic option. Although less efficient than the gemcitabine treatment regimen, cisplatin has also been evaluated for the treatment of patients. Current treatments however only enable a modest improvement in survival at one year compared to untreated patients (18% for gemcitabine, the most efficacious treatment evaluated to date).

Immunotherapy clinical trials have included direct vaccination using whole pancreatic cancer cells, soluble VEGF or EGFR, peptides from MUC1, gastrin, and mesothelin. Indirect immunotherapy approaches have also been tried, e.g., by associating antibodies to VEGF (Bevacizumab) or EGFR (Cetuximab) with drugs such as gemcitabine, tyrosine kinase inhibitors (Erlotinib), microtubule destabilizing agents (Taxotere), or cyclophosphamide (Cytoxan). Such trials are in progress.

Although many monoclonal antibodies have been generated against malignant pancreatic epithelial cells in an effort to identify useful diagnostic and therapeutic markers (e.g., Span-1, Du-Pan-2, CA19-9, CAR-3, CA242, and CO-TL1), few are truly specific for pancreatic tumor cells and their reactivity often depends on the genotype of patients (see, e.g., Kawa et al. (1994) Pancreas; 9:692-7). As such, most markers of this type have failed to offer substantial benefit for the effective diagnosis or treatment of exocrine pancreatic cancer.

In previous studies, it has been shown that human pancreatic tumoral cells overexpress the Feto-Acinar Pancreatic Protein (FAPP), an oncofetal form of the Bile Salt-Dependent Lipase (BSDL) that is associated with a glycosylation change that leads to the specific expression of several glycotopes such as the 16D10 and J28 glycotopes. It has also been shown that there is, at the surface membrane of pancreatic tumoral SOJ-6 cells, a 32 kDa peptide associated with the glycotope that is recognized by monoclonal antibody mAb16D10.

There is thus a great need in the art for new approaches and tools for the treatment of exocrine pancreatic and other forms of cancer. The present invention addresses these and other needs.

### SUMMARY OF THE INVENTION

The present invention results, inter alia, from the surprising discovery that antigen-binding compounds that bind BSDL or FAPP polypeptides are able to induce apoptosis and/or slow the proliferation of tumor cells expressing a BSDL or FAPP polypeptide (it will be appreciated that, as used throughout herein, the phrase “BSDL or FAPP” is not exclusive, i.e. it can also mean “BSDL and/or FAPP” or “BSDL and FAPP”), leading to the death of the cells or halting their growth and proliferation. When apoptosis is triggered by antibody binding to BSDL or FAPP, the resulting programmed cell death is mediated by at least caspase-3, caspase-9, caspase-8 activation and/or poly-ADP ribose polymerase (PARP) cleavage. Further, a decrease of the anti-apoptotic protein Bcl-2 is associated with an increase in the Bax protein, indicating that the caspase activation is controlled by the Bcl-2 family of proteins. As such, the compounds of the invention are particularly useful for inducing the apoptosis of cancer cells, or for treating patients with cancer comprising cancer cells, that express BSDL or FAPP and that are susceptible to apoptosis (e.g., they express caspase-3, -9, -8, PARP, etc). When the compounds of the invention inhibit cell proliferation, it occurs at least by blocking cells at G1/S by, e.g., increasing p53 activity and decreasing cyclin D1 levels, e.g., by activating GSK-3β. Accordingly, the compounds of the invention are particularly useful for halting the proliferation of cancer cells, or for treating patients with cancer comprising cancer cells, that express BSDL or FAPP and that are susceptible to p53 or GSK-3β-mediated G1/S cell cycle arrest.

Importantly, the compounds of the invention are able to directly target tumor cells, particularly BSDL- or FAPP-expressing pancreatic tumor cells, and cause their death via apoptosis and/or halt their proliferation. Significantly, as these effects depend solely on the interaction of the compound with the BSDL or FAPP polypeptide, they can occur even with “naked” compounds (particularly antibodies), i.e. compounds that have not been modified or derivatized with toxic compounds. Further, when the compounds are antibodies, they can effectively target tumor cells even without relying on immune cell mediated killing of the tumor cells (ADCC) (although it should be emphasized that ADCC can also take place in many contexts, further enhancing the efficacy of the treatment). Accordingly, the present compounds are particularly useful for patients with a compromised immune system, e.g., patients with AIDS, patients taking chemotherapy, or patients taking immunosuppressive drug regimens.

Although the compounds of the invention can be any type of molecular entity (e.g. polypeptide, small molecule) that can specifically bind to BSDL- or FAPP-expressing cells and thereby induce their apoptosis or inhibit their growth and proliferation, preferred compounds of the invention are antibodies. Particularly preferred antibodies are bivalent IgG antibodies, as they can typically not only directly decrease target cell number by apoptosis or by inhibiting cell proliferation, but also comprise Fc tails and have sufficient binding affinity to induce the killing of the cells through ADCC. Further, it has been discovered that certain anti-BSDL or anti-FAPP antibodies, particularly multimeric antibodies such as IgM antibodies, tend to be rapidly internalized by BSDL- or FAPP-expressing cells and are thus ineffective at inducing ADCC. Accordingly, by selecting the proper antibodies (bivalent IgG antibodies that target BSDL or FAPP, most preferably the FAPP epitope recognized by antibody 16D10), it is possible to target BSDL- or FAPP-expressing tumor cells through two independent mechanisms (apoptosis induction/cell cycle inhibition and ADCC). Together, these discoveries therefore provide unexpected ways to produce particularly efficacious antigen-binding compounds, most preferably antibodies, that have, inter alia, desired pro-apoptotic or anti-cell proliferation properties as well as, typically, ADCC-inducing effects. Methods of producing and using such antigen-binding compounds, as well as exemplary antigen-binding compounds, are described.

The invention provides methods of using the antigen-binding compounds; for example, the invention provides a method for inducing cell death or inhibiting cell proliferation, comprising exposing a cell, such as a cancer cell which expresses a BSDL or FAPP polypeptide, to an antigen-binding compound that binds a BSDL or FAPP polypeptide in an amount effective to induce cell death or inhibit cell proliferation. It will be appreciated that for the purposes of the present invention, “cell proliferation” can refer to any aspect of the growth or proliferation of cells, e.g., cell growth, cell division, or any aspect of the cell cycle. The cell may be in cell culture or in a mammal, e.g. a mammal suffering from cancer. The invention also provides a method for inducing apoptosis or inhibiting the proliferation of a cell which expresses a BSDL or FAPP polypeptide, comprising exposing the cell to an antigen-binding compound (e.g. exogenous antibody) that binds a BSDL or FAPP polypeptide as described herein in an amount effective to induce apoptosis or inhibit the proliferation of the cell. Thus, the invention provides a method for treating a mammal suffering from a condition characterized by the expression of a BSDL or FAPP polypeptide, e.g., pancreatic cancer, comprising administering a pharmaceutically effective amount of an antigen-binding compound disclosed herein to the mammal. In preferred embodiments, the compound is an antibody, e.g. a bivalent IgG antibody, that is not substantially internalized by BSDL- or FAPP-expressing cells and is thus effective at inducing ADCC of the cells. In preferred embodiments, the antibodies have a half-life of binding to the cell surface of BSDL- or FAPP-expressing cells, e.g., SOJ-6 cells, of at least 40, 60, 80, 100, 120, or more minutes. In other preferred embodiments, the antibodies have a binding affinity to BSDL- or FAPP-epitopes, preferably the epitope specifically recognized by 16D10, of 50, 40, 30, 20, 10, 5, 1, or less nanomolar. The present invention provides methods for producing antigen-binding compounds, particularly antibodies, that specifically bind a BSDL or FAPP polypeptide and that are useful for the treatment of pancreatic cancer. The antigen-binding compounds produced using the present methods are capable of specifically targeting pancreatic tumor cells or any other cells expressing a BSDL or FAPP polypeptide, particularly an epitope on a BSDL and/or FAPP polypeptide recognized by a antibody 16D10. The antigen-binding compound can limit the pathological effects of cell proliferation by inducing apoptosis or inhibiting the proliferation of the cells, as well as optionally by also neutralizing the effects of the expanded cells by virtue of binding alone, by targeting them for destruction by the immune system (e.g., via ADCC), and/or by killing the cells directly by contacting them with a cytotoxic agent such as a radioisotope, toxin, or drug. Methods of using the antigen-binding compounds for the treatment of a BSDL or FAPP polypeptide-expressing cancer (or other conditions associated with the expression of BSDL or FAPP) are also provided. In preferred embodiments, the antibodies have a half-life of binding to the cell surface of BSDL- or FAPP-expressing cells, e.g., SOJ-6 cells, of at least 40, 60, 80, 100, 120, or more minutes. In other preferred embodiments, the antibodies have a binding affinity to BSDL- or FAPP-epitopes, such as the epitope specifically recognized by 16D10, of 50, 40, 30, 20, 10, 5, 1, or less nanomolar. In other embodiments, the antibody is an antibody other than 16D10.

In another embodiment, as the antigen-binding compounds are able to induce the death of tumor cells and/or arrest their growth, the compounds that bind a BSDL and/or FAPP polypeptide can be used in established tumors in order to reduce or limit the volume of such tumors, for example a pancreatic cancer, for example in a tumor which is or which is not able to be resected or debulked surgically, or in a pancreatic cancer where the tumor is established or has spread, for example where the pancreatic cancer is classified as at least a Stage I cancer and/or where the size of the tumor in the pancreas is 2 cm or less in any direction, or where the pancreatic cancer is classified as at least a Stage 2 cancer and/or where the size of the tumor in the pancreas is more than 2 cm in any direction, where the pancreatic cancer is classified as a Stage 2 cancer and/or the cancer has started to grow into nearby tissues around the pancreas, but not inside the nearby lymph nodes, where the pancreatic cancer is classified as a Stage 3 cancer and/or may have grown into the tissues surrounding the pancreas, or where the pancreatic cancer is classified as a Stage 4 cancer and/or has grown into nearby organs. The ability to kill or halt the growth of tumor cells in tumors that have progressed beyond in situ carcinoma is significant in pancreatic cancers, since such cancers are often diagnosed at an advanced stage of development.

Significantly, in certain embodiments, since antigen-binding compounds that bind a BSDL or FAPP polypeptide, particularly in the case when antibodies are used, will not depend exclusively on immune cell mediated cell killing (e.g. ADCC), it is expected that antigen-binding compounds that bind a BSDL or FAPP polypeptide can be used effectively in patients having a deficient or suppressed immune system, and/or in combination with additional anti-tumor agents, particularly therapeutic agents which are known to have adverse impacts on the immune system. For example, immunocompromised patients (e.g., with HIV infection), patients taking immunosuppressive drugs (e.g., subsequent to transplantation or as treatment for autoimmune disorders), or patients taking chemotherapeutic agents are particularly good candidates for treatment with such compounds.

Additionally, since antigen-binding compounds of the invention that bind a BSDL or FAPP polypeptide and have a pro-apoptotic or anti-proliferative effect can eradicate or stop the growth of pancreatic tumor cells, it may be desirable to combine the antigen-binding compounds disclosed herein with other anti-proliferative and/or pro-apoptotic agents in the in vitro and in vivo methods provided herein, such that the respective pro-apoptotic or anti-cell proliferation activities are enhanced, and also so that the cells can be, e.g., first subjected to growth arrest and then eradicated by the pro-apoptotic compounds.

Accordingly, the present invention provides an antigen-binding compound which specifically binds to a BSDL or FAPP polypeptide and which is capable of inducing apoptosis or inhibiting the proliferation of a pancreatic tumor cell. Preferably the antigen-binding compound binds to the same epitope on a BSDL or FAPP polypeptide as antibody 16D10. In one embodiment, the antigen-binding compound competes for binding with antibody 16D10 to a BSDL or FAPP polypeptide (e.g. to an isolated glycopeptide or to a cell expressing it). In one embodiment, the compound is an antibody other than antibody 16D10.

In one embodiment of the methods of the invention, the BSDL or FAPP polypeptide recognized by the antigen-binding compound is a C-terminal peptide of BDSL. In another embodiment, the antigen-binding compound specifically binds a BSDL or FAPP polypeptide comprising or consisting of one or multiple repeated C-terminal peptide sequences of 11 amino acids, comprising a generally invariant part with 7 amino acids having the sequence Ala Pro Pro Val Pro Pro Thr and a glycosylation site. Said generally invariant part is optionally flanked on either side by a glycine often substituted by a glutamic acid and contains the amino acids Asp and Ser on the N-terminal side.

In one embodiment of the methods of the invention, the antigen-binding compound is administered to a subject together with a chemotherapeutic agent. Optionally, the apoptotic effect observed is higher than what would be observed with each drug alone. Optionally, particularly when lower doses of chemotherapeutic agents are used due the combined use of antigen binding compound and chemotherapeutic agent, the growth of tumors cells is arrested, and/or the cell death of tumor cells is increased specifically, e.g. by reducing the effects on (inhibition of proliferation or death) healthy cells. In one embodiment, the chemotherapeutic agent is a tyrosine kinase inhibitor, an alkylating agent or a platinum-based chemotherapy drug. In one embodiment, the chemotherapeutic agent is cisplatin. In one embodiment, the chemotherapeutic agent is a nucleoside analogue, as pyrimidine analogue. In one embodiment, the chemotherapeutic agent is gemcitabine.

In a preferred embodiment, the antigen-binding compound is “naked” and is not functionalized with a radioactive isotope, toxic peptide or toxic small molecule (e.g. a “naked” antibody). In another embodiment, the antigen-binding compound is a cytotoxic antigen-binding compound and comprises an element selected from the group consisting of a radioactive isotope, toxic peptide, and toxic small molecule. In another embodiment, the antigen-binding compound is an antibody that is human, humanized or chimeric. In another embodiment, the radioactive isotope, toxic peptide, or toxic small molecule is directly attached to the antigen-binding compound. In another embodiment, the antigen-binding compound is chemically linked to a chemotherapeutic drug, to form an immunoconjugate, which target selectively the antigen in the diseased cells (see, e.g. U.S. Pat. No. 5,475,092 or U.S. Pat. No. 6,340,701). The antibody-chemotherapeutic agent complexes permit the full measure of the cytotoxic action of the chemotherapeutic agent to be applied in a targeted fashion against unwanted cells only, therefore, avoiding side effects due to damage to non-targeted healthy cells. This invention permits the chemotherapeutic agents to be target site-directed. In another embodiment, the antigen-binding compound is an antibody, e.g., a bivalent chimeric or humanized antibody. In one such embodiment, the antibody comprises the variable (antigen-binding) domains of antibody 16D10. In a preferred embodiment, the antibody comprises an Fc tail. In other preferred embodiments, the antibodies have a half-life of binding to the cell surface of BSDL- or FAPP-expressing cells, e.g., SOJ-6 cells, of at least 40, 60, 80, 100, 120, or more minutes. In other preferred embodiments, the antibodies have a binding affinity to BSDL- or FAPP-epitopes, preferably the epitope specifically recognized by 16D10, of 50, 40, 30, 20, 10, 5, 1, or less nanomolar. In another preferred embodiment, the antibodies are not substantially internalized by BSDL- or FAPP-expressing cells, e.g., SOJ-6 cells, and as such are capable of inducing cell mediated killing (ADCC) of target (BSDL- or FAPP-expressing) cells. In another preferred embodiment, the antibody is hypofucosylated.

Accordingly, the present invention provides a method of treating a patient with pancreatic cancer, the method comprising administering to the patient a pharmaceutically effective amount of an antigen-binding compound according to the invention that specifically binds to a BSDL or FAPP polypeptide, optionally further in combination with a chemotherapeutic agent. The present invention also provides a method of treating a patient, the method comprising a) assessing the pancreatic cancer within the patient, and b) if the cancer is determined to be at a stage where killing of cancer cells, inducing the apoptosis of cancer cells, or inhibiting the growth or proliferation of cancer cells is needed, for example where the cancer is established, surgically treatable, non-surgically treatable, progressed beyond in situ carcinoma, and/or having a diameter of at least 2 cm and/or classified at least Stage 1, administering an antigen-binding compound (e.g., antibody) to the patient that specifically binds a BSDL or FAPP polypeptide and that is capable of inducing the apoptosis of or inhibiting the growth or proliferation of a pancreatic tumor cell, optionally further in combination with a chemotherapeutic agent. In one embodiment, the compound is an antibody, e.g., a bivalent IgG antibody (preferably comprising, in this and other embodiments, an Fc tail), that is not substantially internalized by BSDL- or FAPP-expressing cells and that is capable of inducing cell mediated killing (ADCC) of the cells.

In one embodiment, the invention provides a method of treating a patient having a cancer, a tumor, a pancreatic cancer, comprising administering to said patient an effective dose of an antigen-binding compound of the invention together with a chemotherapeutic agent. In one embodiment, the chemotherapeutic agent is cisplatin. In one embodiment, the chemotherapeutic agent is gemcitabine.

In one embodiment, the invention provides a method of producing an antigen-binding compound suitable for use in the treatment of cancer, said method comprising: i) providing an antigen-binding compound that specifically binds to a BSDL or FAPP polypeptide, ii) testing the ability of the antigen-binding compound for pro-apoptotic or anti-cell proliferation activity; iii) selecting the antigen-binding compound If it is determined that the antigen-binding compound has pro-apoptotic or anti-cell proliferation activity; and optionally iv) producing a quantity of the selected antigen-binding compound. In one embodiment, the compound selected in step iii) is an antibody and is made suitable for human administration prior to step iv), for example by humanizing or chimerizing it. Optionally, a plurality of antigen-binding compounds are provided in step i), and they are each tested in step ii) for their ability to induce apoptosis or inhibit the proliferation of a cell expressing a BSDL or FAPP polypeptide. Typically, step ii) will involve standard assays in which cells, e.g. BSDL- or FAPP-expressing cells, preferably tumor cells such as SOJ-6 cells or cells taken from a patient with pancreatic cancer, will be contacted with the compound and the proliferation or survival of the cells will be assessed, often in conjunction with an analysis of the activity of known apoptosis or cell growth/cycle regulatory genes. In one embodiment, the method further comprises a step of assessing whether a chemotherapeutic compound enhances the pro-apoptotic or anti-cell proliferation activity of the antigen-binding compound. Optionally, the latter step comprises conducting assays in which cells, e.g. BSDL- or FAPP-expressing cells, preferably tumor cells such as SOJ-6 cells or cells taken from a patient with pancreatic cancer, are contacted with the chemotherapeutic compound and the antigen-binding compound, and the proliferation or survival of the cells is assessed. A determination that the chemotherapeutic compound enhances the pro-apoptotic or anti-cell proliferation activity of the antigen-binding compound indicates that the chemotherapeutic compound is suitable for use in combination with the antigen-binding compound.

In another embodiment, the invention provides a method of producing an antibody suitable for use in the treatment of cancer, said method comprising: i) providing an antibody that specifically binds to a BSDL or FAPP polypeptide, ii) testing the antibody for pro-apoptotic or anti-cell proliferation activity; iii) testing the antibody for the ability to induce immune cell mediated killing (ADCC) of cells, e.g., tumor cells, expressing BSDL or FAPP; iv) selecting the antibody if it is determined that the antigen-binding compound has pro-apoptotic or anti-cell proliferation activity and is capable of inducing ADCC of cells, e.g., tumor cells, expressing BSDL or FAPP; and optionally v) producing a quantity of the selected antigen-binding compound. In one embodiment, the antibody selected in step iv) is made suitable for human administration prior to step v), for example by humanizing or chimerizing it. Optionally, a plurality of antigen-binding compounds are provided in step i), and they are each tested in step ii) for their ability to induce apoptosis or inhibit the proliferation of cells expressing a BSDL or FAPP polypeptide. In preferred embodiments, the antibody is IgG. Additionally, the antibody is preferably bivalent. In another preferred embodiment, the antibody does not cross-react with non-tumor tissues selected from the group consisting of tonsils, salivary gland, peripheral nerve, lymph node, eye, bone marrow, ovary, oviduct, parathyroid, prostate, spleen, kidney, adrenals, testis, thymus, ureters, uterus, and bladder.

In another embodiment, the invention provides a method of producing an antibody suitable for use in the treatment of cancer, said method comprising: i) providing an antibody that specifically binds to a BSDL or FAPP polypeptide, ii) testing the antibody for pro-apoptotic or anti-cell proliferation activity; iii) testing the internalization of the antibody by cells, e.g., tumor cells, expressing BSDL or FAPP; iv) selecting the antibody if it is determined that the antigen-binding compound has pro-apoptotic or anti-cell proliferation activity and is not substantially internalized by cells, e.g., tumor cells, expressing BSDL or FAPP; and optionally v) producing a quantity of the selected antigen-binding compound. In one embodiment, the antibody selected in step iv) is made suitable for human administration prior to step v), for example by humanizing or chimerizing it. Optionally, a plurality of antigen-binding compounds are provided in step i), and they are each tested in step ii) for their ability to induce apoptosis or inhibit the proliferation of cells expressing a BSDL or FAPP polypeptide. In preferred embodiments, the antibody is of IgG isotype. Additionally, the antibody is preferably bivalent (and comprises an Fc tail). In another preferred embodiment, the antibody does not cross-react with non tumor tissues selected from the group consisting of tonsils, salivary gland, peripheral nerve, lymph node, eye, bone marrow, ovary, oviduct, parathyroid, prostate, spleen, kidney, adrenals, testis, thymus, ureters, uterus, and bladder. In preferred embodiments, the antibodies have a half-life of binding to the cell surface of BSDL- or FAPP-expressing cells, e.g., SOJ-6 cells, of at least 40, 60, 80, 100, 120, or more minutes. In other preferred embodiments, the antibodies have a binding affinity to BSDL- or FAPP-epitopes, preferably the epitope specifically recognized by 16D10, of 50, 40, 30, 20, 10, 5, 1, or less nanomolar. In other preferred embodiments, the antibody is hypofucosylated.

In another embodiment, the invention provides a method of producing an antigen-binding compound suitable for use in the treatment of cancer, said method comprising: i) producing a quantity of an antigen-binding compound that specifically binds to a BSDL or FAPP polypeptide, ii) testing a sample from said quantity of antigen-binding compound for pro-apoptotic or anti-cell proliferation activity; iii) selecting the quantity for use as a medicament and/or in the manufacture of a medicament if it is determined that the antigen-binding compound has pro-apoptotic or anti-cell proliferation activity; and optionally iv) preparing the quantity for administration to a human, optionally formulating a quantity of the selected antigen-binding compound with a pharmaceutically acceptable carrier.

In another embodiment, the invention provides a method of producing an antigen-binding compound suitable for use in the treatment of cancer, said method comprising: i) providing a plurality of antigen-binding compounds that specifically bind to a BSDL or FAPP polypeptide; ii) testing the ability of each of the antigen-binding compounds for pro-apoptotic or anti-cell proliferation activity; iii) selecting an antigen-binding compound capable of inducing apoptosis or inhibiting the proliferation of said cell; and iv) optionally, making the antigen-binding compound suitable for human administration; and/or optionally v) producing a quantity of the selected antigen-binding compound. In one embodiment, the method comprises an additional step in which the compound is an antibody, and the internalization of the antibody by cells expressing BSDL or FAPP is assessed, wherein a finding that the antibody selected in step iii) is not substantially internalized by cells expressing BSDL or FAPP confirms its suitability for use in the treatment of cancer. In another embodiment, the method comprises an additional step in which the compound is an antibody, and the ability of the antibody to induce the cell-meditated killing (ADCC) of cells, e.g., tumor cells, expressing BSDL or FAPP is assessed, wherein a finding that the antibody selected in step iii) is able to induce ADCC of cells, e.g., tumor cells, expressing BSDL or FAPP confirms its suitability for use in the treatment of cancer.

In another embodiment, the invention provides a method of producing an antigen-binding compound, comprising: i) providing an antigen-binding compound that specifically binds to tumor cells expressing a BSDL or FAPP polypeptide taken from one or more patients with pancreatic cancer; ii) testing the antigen-binding compound for pro-apoptotic or anti-cell proliferation activity towards tumor cells taken from one or more patients with pancreatic cancer; iii) if the antigen-binding compound induces apoptosis or inhibits the proliferation of a substantial number of tumor cells taken from one or more of the patients, making the antigen-binding compound suitable for human administration; and iv) optionally producing a quantity of the human-suitable antigen-binding compound. In one embodiment, the method comprises an additional step in which the compound is an antibody, and the internalization of the antibody by cells expressing BSDL or FAPP is assessed, wherein a finding that the antibody used in step iii) is not substantially internalized by cells expressing BSDL or FAPP confirms its suitability for use in the treatment of pancreatic cancer. In another embodiment, the method comprises an additional step in which the compound is an antibody, and the ability of the antibody to induce the cell-meditated killing (ADCC) of tumor cells expressing BSDL or FAPP is assessed, wherein a finding that the antibody used in step iii) is able to induce ADCC of tumor cells expressing BSDL or FAPP confirms its suitability for use in the treatment of pancreatic cancer.

In one embodiment of any of the methods of the invention, the method may comprise a step of immunizing a non-human mammal (e.g. a mouse, rat, rabbit, mouse transgenic for human Ig genes, etc.) with a BSDL or FAPP polypeptide prior to step i). In another embodiment, the method comprises a step of generating a library of antigen-binding compound (e.g. via phage display methods and the like) and selecting an antigen-binding compound that binds BSDL or FAPP polypeptide prior to step i).

In one embodiment of any of the methods of the invention, the antigen-binding compound or antibody of step i) and/or step ii) does not comprise a cytotoxic agent such as a radioactive isotope, a toxic polypeptide, or a toxic small molecule.

Testing the ability of each of the antigen-binding compound or antibodies to induce apoptosis of a cell or to inhibit its proliferation can be carried out according to any of a variety of available methods. For example, said testing may comprise without limitation detecting death of a target cell (e.g. tumor cell, SOJ-6 cell, cell expressing a BSDL or FAPP polypeptide), detecting nuclear fragmentation, detecting activity (e.g. caspase activation) or increases and/or decreases in levels of protein involved in apoptosis. Similarly, testing for compounds affecting the growth or proliferation of cells can be carried out according to any of a variety of available methods, e.g., counting cell number, density, DNA replication, mitotic index, measuring levels of proteins or other molecules involved in cell growth or proliferation, or any other measure of cell growth or proliferation.

In one embodiment of any of the methods of the invention, testing for pro-apoptotic or anti-cell proliferation activity comprises determining whether an antigen-binding compound induces apoptosis or inhibits the growth or proliferation of a cell expressing a BSDL or FAPP polypeptide. Optionally, the cell expresses a BSDL or FAPP polypeptide in a lipid raft. Optionally, the cell is made to express a BSDL or FAPP polypeptide. Optionally, the cell is a tumor cell line. Optionally, the cell is a pancreatic cancer cell, optionally a SOJ-6 cell. Optionally, the cell is a tumor cell taken from one or more patients with cancer, e.g., exocrine pancreatic cancer.

In one embodiment of any of the methods of the invention, determining whether an antigen-binding compound induces apoptosis or inhibits the proliferation of a cell expressing a BSDL or FAPP polypeptide can be carried out in the absence of immune effector cells, particularly NK cells.

In one embodiment of any of the methods of the invention, testing for pro-apoptotic or anti-cell growth or proliferation activity comprises determining whether an antigen-binding compound modulates the activity or level of an apoptotic or cell proliferation regulatory protein or marker in a cell expressing a BSDL or FAPP polypeptide. Preferably, for apoptosis the regulatory protein is a caspase or a Bcl-2 family member. For cell growth or proliferation, the regulatory protein or marker can be, e.g., PCNA, Ki-67, cyclin such as cyclin D (e.g., cyclin D1, E2F, Rb, p53, MCM6, GSK-3β, Bcl10, or BrdU incorporation.

In one embodiment of any of the methods of the invention, making the antigen-binding compound suitable for administration to a human comprises making an anti-BSDL or FAPP antibody chimeric, human, or humanized. Making the compound suitable for administration to a human can also comprise formulating the compound with a pharmaceutically acceptable carrier.

In one embodiment of any of the methods of the invention, producing a quantity of antigen-binding compound comprises culturing a cell expressing the antigen-binding compound in a suitable medium and recovering the antigen-binding compound. Optionally, the cell is a recombinant host cell made to express the antigen-binding compound. In one embodiment, the compound is a monoclonal antibody and the cell is a hybridoma.

In one embodiment of any of the methods of the invention, the antigen-binding compound, particularly the antigen-binding compound produced by the method does not comprise a cytotoxic agent such as a radioactive isotope, a toxic polypeptide, or a toxic small molecule. In one embodiment, the antigen-binding compound is an antibody that specifically binds a BSDL or FAPP polypeptide. In one embodiment of any of the methods of the invention, the antigen-binding compound competes for binding with antibody 16D10 to a BSDL or FAPP polypeptide. In one embodiment of any of the methods of the invention, the compound is an antibody other than 16D10. In another embodiment of any of the methods of the invention the compound is a chimeric, human, or humanized version of antibody 16D10.

In one embodiment of any of the methods of the invention, the antigen-binding compound, preferably an antibody, has an Fc receptor binding portion, preferably a heavy chain constant region of an IgG isotype, optionally of a human IgG isotype. In a preferred embodiment, the antibody is an IgG1 antibody. The invention also encompasses fragments and derivatives of antibodies having substantially the same antigen specificity and activity (e.g., which can bind to the same antigens as the parent antibody). Such fragments include, without limitation, Fab fragments, Fab′2 fragments, CDR and ScFv. When the compound is an antibody, the antibody will typically be, for example, chimeric, humanized or human. In one preferred embodiment, the antibody is a recombinant chimeric antibody. In one such embodiment, the domains Cu2, Cu3, and Cu4 of the mouse heavy chain of an anti BSDL or FAPP antibody, e.g., 16D10, is replaced by a human IgG, e.g. IgG1. In another preferred embodiment, the antibody is a chimeric antibody in which the constant regions of a mouse anti-BSDL or FAPP antibody, e.g., 16D10, are replaced by human IgG1 constant regions for both heavy and light chains.

In certain embodiments, the compounds of the invention are multimeric (i.e. cross-linked) IgG antibodies. In preferred embodiments, the antibodies are tetrameric (two heavy and two light chains) and are thus bivalent. In particularly preferred embodiments, the antibodies are capable of inducing apoptosis or inhibiting the proliferation of tumor cells expressing BSDL or FAPP. In particularly preferred embodiments, the antibodies are capable of inducing apoptosis or inhibiting the proliferation of tumor cells expressing BSDL or FAPP, and are also not substantially internalized by cells expressing BSDL or FAPP. In other particularly preferred embodiments, the antibodies are capable of inducing apoptosis or inhibiting the proliferation of tumor cells expressing BSDL or FAPP, and are also able to induce the cell mediated killing (ADCC) of cells expressing BSDL or FAPP. In other particularly preferred embodiments, the antibodies do not cross react with tissues selected from the group consisting of tonsils, salivary gland, peripheral nerve, eyes, bone marrow, ovaries, oviducts parathyroid gland, prostate, spleen, kidney, adrenal glands, testes, thymus, ureters, uterus, and bladder. In other preferred embodiments, the antibodies have a half-life of binding to the surface of BSDL- or FAPP-expressing cells, e.g., SOJ-6 cells, of at least 40, 50, 60, 70, 80, 100, 120, 150, 200, or more minutes. In other preferred embodiments, the antibodies have a binding affinity to a BSDL or FAPP epitope (e.g., the epitope recognized by 16D10) of 50, 40, 30, 20, 10, 5, 1, or less nanomolar.

In another embodiment, the invention encompasses an antigen-binding compound produced according to any of the methods of the invention.

The invention also encompasses pharmaceutical formulations comprising any of the antigen binding compounds and in particular any of the antibodies of the invention and a pharmaceutically acceptable carrier are also provided, as are kits. Kits may for example comprise the compound and instructions for its use, e.g., in the treatment of pancreatic cancer. Kits may comprise the compound and a carrier; kits may comprise the compound in a manufactured (e.g. glass, plastic or other) container. Cells expressing the antibodies, e.g., hybridomas, are also encompassed. In another embodiment, the kit comprises an antigen-binding compound and a chemotherapeutic agent, and instructions for combined use.

In one embodiment, the antigen-binding compound or antibody of the invention competes for binding with antibody 16D10 to a BSDL or FAPP polypeptide. The invention also encompasses fragments and derivatives of the antibodies having substantially the same antigen specificity and activity as antibody 16D10 (e.g., which can bind to the same antigens as the parent antibody). Such fragments include, without limitation, Fab fragments, Fab′2 fragments, CDR and ScFv.

In one embodiment, the composition and/or methods of the inventions specifically exclude the antibody 16D10, particularly the IgM antibody 16D10 produced by the cell deposited with the Collection Nationale de Culture de Microorganismes (CNCM) in Paris on 16 Mar. 2004 under the number I-3188.

Accordingly, in another embodiment, the invention provides an antibody, preferably an isolated antibody, which binds to a BSDL or FAPP polypeptide and which is capable of inducing apoptosis or inhibiting the proliferation of a cell which expresses a BSDL or FAPP polypeptide, wherein the antibody competes for binding with antibody 16D10 to a BSDL or FAPP polypeptide, and wherein the antibody is not 16D10.

In another embodiment, the invention provides a bivalent antibody comprised of two heavy chains and two light chains, wherein the heavy chains comprise an IgG heavy chain constant region capable of binding to an Fc receptor, and wherein the antibody: (a) is capable of inducing apoptosis or inhibiting the proliferation of cells expressing a BSDL or FAPP polypeptide; (b) is capable of inducing cell-mediated killing (ADCC) of BSDL- or FAPP-expressing cells; and (c) competes for binding with antibody 16D10 to a BSDL or FAPP polypeptide.

In another embodiment, the invention provides a bivalent antibody comprising: (a) a heavy chain comprising a variable region comprising one or more CDRs derived from the amino acid sequence of SEQ ID NO: 7 fused to a human IgG chain constant region; and (b) a light chain comprising a variable region comprising one or more CDRs derived from the amino acid sequence of SEQ ID NO: 8, optionally fused to human kappa chain constant region.

Optionally, any of the antibodies herein can further be characterized by not being substantially internalized by BSDL or FAPP-expressing cells. In another embodiment, any of the antibodies herein can further be characterized by also being capable of inducing the cell mediated killing (ADCC) of BSDL- or FAPP-expressing cells. any of the antibodies herein can further be characterized as not comprising a cytotoxic agent such as a radioactive isotope, a toxic polypeptide, or a toxic small molecule. Any of the antibodies herein can further be characterized by being capable of inducing apoptosis or inhibiting the proliferation of a pancreatic tumor cell. any of the antibodies herein can further be characterized by being capable of modulating the activity or level of an apoptotic regulatory protein in a cell expressing a BSDL or FAPP polypeptide. In another embodiment, any of the antibodies herein can further be characterized by being capable of modulating the activity or level of a caspase or a Bcl-2 family member. In another embodiment, the antibody modulates the activity or level of a cell proliferation or growth regulatory protein in a cell expressing a BSDL or FAPP polypeptide. In another embodiment, the antibody modulates the activity or level of a cell proliferation or growth regulatory protein in a BSDL- or FAPP-expressing cell selected from the group consisting of GSK-3β, cyclin D1, and p53. In another embodiment, any of the antibodies herein can further be characterized as having a heavy chain constant region of an IgG isotype, optionally of a human IgG or IgG1 isotype. In another embodiment, any of the antibodies herein can further be characterized by being tetrameric. In another embodiment, any of the antibodies herein can further be characterized as being bivalent. In another embodiment, any of the antibodies herein can further be characterized as being a chimeric, human or humanized antibody. In another embodiment, any of the antibodies herein can further be characterized as being hypofucosylated.

In one embodiment of any of the herein-described antibodies, the antibody binds to the surface of BSDL- or FAPP-expressing cells with a half-life of at least 40, 60, 80, 100, 120, 180, 240, or more minutes. In another embodiment, the antibody binds to the BSDL or FAPP epitope with a binding affinity of at least 50, 40, 30, 20, 10, 5, or 1 nanomolar.

The invention also encompasses a pharmaceutical composition comprising any of the herein-described antigen-binding compounds or antibodies, and a pharmaceutically acceptable carrier. In another aspect, the invention encompasses a kit comprising an antigen-binding compound or an antibody of the invention, and instructions for using said antigen-binding compound or antibody in the treatment or diagnosis of a pancreatic or FAPP or BSDL expressing pathology, e.g. pancreatic cancer. In another embodiment, cells, e.g., hybridomas, are also provided.

In other aspects, provided is a method of inducing the apoptosis or inhibiting the proliferation of a cancer cell, and/or of treating a patient or individual with a cancer, the method comprising: a) determining if the cancer or cancer cell is suitable for treatment with a pro-apoptotic or anti-cell proliferation agent, and b) in the case of a positive determination that the cancer or cancer cell is suitable for treatment with a pro-apoptotic or anti-cell proliferation agent, contacting the cancer cell with an effective amount of any of the antigen-binding compounds of the invention. In yet another aspect, the invention provides a method of inducing the apoptosis or inhibiting the proliferation of a cancer cell, and/or of treating a patient with a cancer, the method comprising: a) determining if the cancer or cancer cell expresses a BSDL or FAPP polypeptide, and b) in the case of a positive determination that the cancer or cancer cell expresses a BSDL and/or FAPP polypeptide, contacting the cancer cell with an effective amount of an antigen-binding compound as disclosed herein. Optionally, in these methods, the step of contacting the cancer cell comprises administering to the patient a pharmaceutically effective amount of an antigen-binding compound of the invention. Preferably, the pharmaceutically effective amount is an amount effective to induce apoptosis or inhibit the proliferation of cancer cell(s) in the patient. Also optionally in these methods, the compound is an antibody, and the methods involve an additional step in which the internalization of the antibody by BSDL- or FAPP-expressing cells is assessed, or in which the ability of the antibody to induce cell-mediated killing (ADCC) of BSDL- or FAPP-expressing cells is assessed, wherein a determination that the antibody is either not substantially internalized or is capable of inducing cell-mediated killing of BSDL- and/or FAPP-expressing cells indicates that the antibody is suitable for use in step b). In certain embodiments, the contacting is carried out in the absence or relative paucity of immune effector cells, e.g., NK cells, for example when such methods are carried out in vitro or when they are carried out in patients with deficient immune systems (e.g., due to conditions such as AIDS, to conditions that decrease NK cell levels, to the administration of chemotherapeutic agents, or to the use of immunosuppressive agents, for example in conjunction with a transplantation procedure or treatment of autoimmune disorders).

In another aspect, the invention provides a method of decreasing tumor volume in a patient, comprising administering to the patient a pharmaceutically effective amount of an antigen-binding compound of the invention.

In another aspect, the invention provides a method of inducing the apoptosis of or inhibiting the proliferation of a BSDL or FAPP polypeptide-expressing cell, optionally of a tumor cell, comprising bringing said cell into contact with an antigen-binding compound of the invention in an amount effective to induce apoptosis or inhibit the proliferation of the cell. Optionally, said bringing into contact is in the absence or relative paucity of immune effector cells, e.g., NK cells, and/or is carried out in vitro. Optionally the method further comprises determining whether the antigen-binding compound is capable of inducing apoptosis or inhibiting the proliferation of the cell. Optionally, the compound is an antibody that is not substantially internalized by BSDL- or FAPP-expressing cells and/or is capable of inducing the cell-mediated killing (ADCC) of BSDL- or FAPP-expressing cells, and said bringing into contact is in the presence of immune effector (e.g., NK) cells.

### DESCRIPTION OF THE FIGURES

FIG. 1 demonstrates the ability of mAb16D10 to stimulate apoptotic cellular death of SOJ-6 cells (compared to RPMI and mouse IgM; the y-axis represents the number of apoptotic cells/cm2).

FIG. 2 shows apoptosis induction by 16D10 as measured with CaspAce FITC-VAD-fmk on Pancreatic SOJ-6 cells pre-treated with or without caspase inhibitors (caspase 9: LEHD-fmk, caspase8: Z-IEDT-fmk, caspase3: Z-DEVED-fink, and caspase mix: Z-VAD-fmk), and then treated with mAb16D10; mAb16D10 stimulates apoptosis through caspase-3, caspase-8, and caspase-9.

FIG. 3 shows apoptosis of SOJ-6 cells induced by mAb16D10 as observed by DAPI staining; RPMI induced no apoptosis on cells, Cisplatin induced a low level of apoptosis, and antibody 16D10 induced significant levels of apoptosis.

FIG. 4 shows the results on a gel, demonstrating that treatment of cells with 16D10 induces a decrease of the anti-apoptotic protein Bcl-2 associated with an increase of Bax protein, indicating that the caspase activation is controlled by the Bcl-2 family of proteins. The experiment also demonstrated that 16D10 induced apoptosis is mediated via caspases 8 and 9, and poly-ADP ribose polymerase (PARP) cleavage. The leftmost lane represents SOJ-6 cells in RPMI, the middle lane represents SOJ-6 cells incubated with antibody 16D10, and the rightmost lane represents SOJ-6 cells incubated with cisplatin.

FIG. 5 shows the results of an MTT assay involving treatment of SOJ-6 pancreatic tumor cells with increasing concentrations of polyclonal antibody pAbL64 which recognizes human BDSL/FAPP. pAbL64 is unable to cause a decrease in growth or number of cells (x-axis is mAb concentration and y-axis is % growth of cells).

FIG. 6 shows the results of an MTT assay involving the treatment of SOJ-6 pancreatic tumor cells with increasing concentrations of polyclonal antibody J28 which recognizes human BDSL/FAPP but which has been demonstrated by the inventors to bind a different epitope on BDSL/FAPP from antibody 16D10. J28 is unable to cause a decrease in growth or number of cells (x-axis is mAb concentration and y-axis is % growth of cells).

FIG. 7 shows the results of an MTT assay involving the treatment of SOJ-6 or PANC-1 pancreatic tumor cells with increasing concentrations of polyclonal antibody 16D10 (IgM) which recognizes human BDSL/FAPP. FIG. 7 shows that 16D10 is unable to cause a decrease in growth or number of PANC-1 cells which do not express 16D10 antigen but does cause a decrease in SOJ-6 cells which do express FAPP (x-axis is mAb concentration and y-axis is % growth of cells).

FIG. 8 shows the results of an MTT assay involving the treatment of SOJ-6 or PANC-1 pancreatic tumor cells with increasing concentrations of a control IgM antibody showing that control IgM antibody is unable to cause a decrease in the growth or number of either PANC-1 or SOJ-6 cells (x-axis is mAb concentration and y-axis is % growth of cells).

FIG. 9 shows the results of an MTT assay involving the treatment of SOJ-6 pancreatic tumor cells with increasing concentrations of either antibody 16D10 or control IgM antibody, demonstrating that 16D10 causes a decrease in cells while the control IgM antibody does not (x-axis is mAb concentration and y-axis is % growth of cells).

FIG. 10 shows the results of an MTT assay involving the treatment of SOJ-6 pancreatic tumor cells with increasing concentrations of either antibody 16D10 or a control IgM antibody, and methyl-b-cyclodextrin (MBCD) at various concentrations with or without antibody 16D10; MBCD when used in combination with 16D10 decreases or abolishes the cell growth inhibiting activity of antibody 16D10. This data indicate that the ability of mAb16D10 to stimulate apoptotic cellular death is dependent of the localization of the 16D10 antigen in membrane lipid RAFT microdomains.

FIG. 11: mAb16D10 arrests cell cycle progression in G1/S phase and regulates the expression of p53, cyclin D1, and GSK-3β. Equal amounts of cell lysates (50 μg) were loaded on SDS-PAGE, transferred to nitrocellulose, and probed with specific antibodies (p53, cyclin D1, phospho-GSK-3β and GSK-3β after treatment with mAb16D10. β-actin was used as an internal control. Each experiment was carried out in triplicate.

FIGS. 12A-12B: Disorganization of membrane rafts structure decreases the mAb16D10 effect. SOJ-6 cells were seeded at 8000 cells/well and grown overnight. The culture medium was replaced by fresh medium containing methyl-β-cyclodextrin (MβCD) or Filipin (FIG. 12A) or metabolic inhibitors of glycosphingolipid biosynthesis (FIG. 12B) for 6 h and was then replaced by fresh medium with inactivated FBS containing with antibodies. Cell viability was determined by MTT assays. Results are represented as mean±SD of three independent experiments.

FIG. 13: Effect of mAb16D10 treatment on E-cadherin/β-catenin complex in SOJ-6 and PANC-1 cells. SOJ-6 cells were treated with or without mAb16D10 at 25 μg/ml for 24 h. Equal amounts of cell lysates (50 μg) were resolved by SDS-PAGE, transferred to nitrocellulose, and probed with specific antibodies (anti-phospho-β-catenin, anti-β-catenin, anti-E-cadherin and anti-β-actin).

FIG. 14 shows the results of flow cytometry demonstrating that antibody 16D10 was found to bind antigen found on SOJ-6 cells. The x-axis shows fluorescent intensity and the y-axis shows counts.

FIG. 15 shows the results of flow cytometry demonstrating that antibody 16D10 did not bind antigen found on PANC-1 cells. The x-axis shows fluorescent intensity and the y-axis shows counts.

FIG. 16 shows the strategy used in the production of a bivalent 16D10 chimeric antibody in HEK293T cells.

FIG. 17 shows the 16D10 VH and VL cloning strategy, including the VH, CH1, IgG1-Fc, and VL and Ck sequences.

FIG. 18 shows the effects of 16D10 and Rec16D10 treatment on SOJ-6 cell proliferation.

FIG. 19 shows the strategy used to test Rec16D10 mediated NK cell activation.

FIG. 20 shows the induction of CD107 mobilization by Reel 6D10 on NK cells.

FIG. 21 shows the induction of IFN-γ secretion by Rec16D10 on NK cells.

FIG. 22 shows the results of a Tissue Cross-Reaction Study using Rec16D10 and other antibodies.

FIG. 23 shows apoptosis of SOJ-6 cells induced by recombinant chimeric IgG1 16D10 antibody, as observed by Annexin V and V/PI staining; cells by themselves underwent no or low apoptosis, and each of tunicamycin, IgM antibody 16D10 and IgG1 antibody 16D10 induced significant levels of apoptosis.

FIG. 24 shows the sequence of the VH-16D10-HuIgG1 and VL16D10-HuIgL Kappa, respectively. The CDRs 1, 2 and 3 are shown in bold for each sequences. The variable region sequences are underlined for each sequence, with the remaining sequences corresponding to constant region sequences of the human IgG1 and kappa type, respectively.

FIG. 25A shows that the proliferation (represented as percentage in ordinate) of pancreatic cancer cells (SOJ-6 cells, black histograms) is impaired when treated with gemcitabine, compared to untreated cells. The pancreatic cancer cell line PANC-1 (light histograms) is relatively resistant to treatment. FIG. 25B shows that the proliferation (represented as percentage in ordinate) of pancreatic cancer cells (SOJ-6 cells, black histograms) is impaired when treated with cisplatin, compared to untreated cells. The pancreatic cancer cell line PANC-1 (light histograms) is relatively resistant to treatment.

FIG. 26A shows that the proliferation (represented as percentage in ordinate) of SOJ-6 pancreatic cancer cells (SOJ-6 cells, black histograms; PANC-1, light histograms) is impaired when treated first with gemcitabine or cisplatin, followed by antibody 16D10, compared to cells treated with gemcitabine or cisplatin alone. FIG. 26B shows that the proliferation (represented as percentage in ordinate) of SOJ-6 pancreatic cancer cells (SOJ-6 cells, black histograms; PANC-1, light histograms) is impaired when treated first with antibody 16D10, followed by gemcitabine or cisplatin, compared to cells treated with antibody 16D10 alone.

FIG. 27 shows that apoptosis (represented as percentage of apoptotic cells per cm² in ordinate) is clearly enhanced with antibody 16D10 and the combination of antibody 16D10 and chemotherapeutic agents gemcitabine or cisplatin (SOJ-6 cells in black histograms; PANC-1 cells in light histograms).

### DETAILED DESCRIPTION OF THE INVENTION

### Definitions

As used herein, the following terms have the meanings ascribed to them unless specified otherwise.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). As such, tetramers, e.g., IgG tetramers, are “bivalent” as they have two antigen recognition sites. Such bivalent tetramers, particularly IgG tetramers, are preferred in the present invention as they are capable of conveying both anti-proliferation/pro-apoptotic activity and of inducing ADCC of target cells (so long as the antibodies comprise an Fc tail and are thus capable of binding to Fc receptors). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM are the preferred classes of antibodies employed in this invention, with IgG being particularly preferred, because they are the most common antibodies in the physiological situation and because they are most easily made in a laboratory setting. Further, it has been discovered that multimeric antibodies such as IgM antibodies are more rapidly internalized than tetrameric forms such as IgG tetramers, and as such are less effective at inducing immune cell mediated targeting (via ADCC) of tumor cells. IgG tetramers are also more specific, i.e. have less non-specific binding, than multimeric IgM antibodies. Preferably the antibodies of this invention are monoclonal antibodies. Particularly preferred are humanized, chimeric, human, or otherwise-human-suitable antibodies. “Antibodies” also includes any fragment or derivative of any of the herein described antibodies.

The term “specifically binds to” means that an antigen-binding compound or antibody can bind preferably in a competitive binding assay to the binding partner, e.g. a BSDL or FAPP polypeptide, as assessed using either recombinant forms of the proteins, epitopes therein, or native proteins present on the surface of relevant target cells (e.g. tumor cells, SOJ-6 cells, etc.). Competitive binding assays and other methods for determining specific binding are further described below and are well known in the art.

A “human-suitable” antibody refers to any antibody, derivatized antibody, or antibody fragment that can be safely used in humans for, e.g. the therapeutic methods described herein. Human-suitable antibodies include all types of humanized, chimeric, or fully human antibodies, or any antibodies in which at least a portion of the antibodies is derived from humans or otherwise modified so as to avoid the immune response that is generally provoked when native non-human antibodies are used.

As used herein, the terms “conjoint”, “in combination” or “combination therapy”, used interchangeably, refer to the situation where two or more agents (e.g. an antigen-binding compound of the invention and a chemotherapeutic agent) affect the treatment or prevention of the same disease. The use of the terms “conjoint”, “in combination” or “combination therapy” do not restrict the order in which the agents are administered to a subject with the disease. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject with a disease.

As used herein, the term “synergistic” or “synergy” refers to a combination of therapeutic agents which is more effective than the additive effects of any two or more single agents. For example, a synergistic effect of a combination of therapeutic agents permits the use of lower dosages of one or more of the agents and/or less frequent administration of said therapies to a subject with cancer. The ability to utilize lower dosages of therapeutic agents and/or to administer said therapies less frequently reduces the toxicity associated with the administration of said therapies to a subject without reducing the efficacy of said therapies in the prevention or treatment of cancer. In addition, a synergistic effect can result in improved efficacy of therapies in the prevention or treatment of cancer. Finally, synergistic effect of a combination of therapies may avoid or reduce adverse or unwanted side effects associated with the use of any single therapy.

As used herein, the term “therapeutically effective amount” refers to that amount of a therapeutic agent which is sufficient to reduce or ameliorate the severity, duration and/or progression of a disease or one or more symptoms thereof. For example, when referring to cancer, a therapeutically effective amount may refer to that amount which is sufficient to destroy, modify, control or remove primary, regional or metastatic cancer tissue, ameliorate cancer or one or more symptoms thereof, or prevent the advancement of cancer, cause regression of cancer, or enhance or improve the therapeutic effect(s) of another therapeutic agent. A therapeutically effective amount, when referring to cancer, may refer to the amount of a therapeutic agent sufficient to delay or minimize the spread of cancer. A therapeutically effective amount may also refer to the amount of a therapeutic agent that provides a therapeutic benefit in the treatment or management of cancer. Further, a therapeutically effective amount with respect to a therapeutic agent of the invention means that amount of therapeutic agent alone, or in combination with other therapies, that provides a therapeutic benefit in the treatment or management of cancer.

“Toxic” or “cytotoxic” peptides or small molecules encompass any compound that can slow down, halt, or reverse the proliferation of cells, decrease their activity in any detectable way, or directly or indirectly kill them. Preferably, toxic or cytotoxic compounds work by directly killing the cells, by provoking apoptosis or otherwise. As used herein, a toxic “peptide” can include any peptide, polypeptide, or derivative of such, including peptide- or polypeptide-derivatives with unnatural amino acids or modified linkages. A toxic “small molecule” can includes any toxic compound or element, preferably with a size of less than 10 kD, 5 kD, 1 kD, 750 D, 600 D, 500 D, 400 D, 300 D, or smaller.

By “immunogenic fragment”, it is herein meant any polypeptidic or peptidic fragment which is capable of eliciting an immune response such as (i) the generation of antibodies binding said fragment and/or binding any form of the molecule comprising said fragment, including the membrane-bound receptor and mutants derived therefrom, (ii) the stimulation of a T-cell response involving T-cells reacting to the bi-molecular complex comprising any MHC molecule and a peptide derived from said fragment, (iii) the binding of transfected vehicles such as bacteriophages or bacteria expressing genes encoding mammalian immunoglobulins. Alternatively, an immunogenic fragment also refers to any construction capable of eliciting an immune response as defined above, such as a peptidic fragment conjugated to a carrier protein by covalent coupling, a chimeric recombinant polypeptide construct comprising said peptidic fragment in its amino acid sequence, and specifically includes cells transfected with a cDNA whose sequence comprises a portion encoding said fragment.

For the purposes of the present invention, a “humanized” antibody refers to an antibody in which the constant and variable framework region of one or more human immunoglobulins is fused with the binding region, e.g. the CDR, of an animal immunoglobulin. Such humanized antibodies are designed to maintain the binding specificity of the non-human antibody from which the binding regions are derived, but to avoid an immune reaction against the non-human antibody.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “human” antibody is an antibody obtained from transgenic mice or other animals that has been “engineered” to produce specific human antibodies in response to antigenic challenge (see, e.g., Green et al. (1994) Nature Genet. 7:13; Lonberg et al. (1994) Nature 368:856; Taylor et al. (1994) Int Immun 6:579, the entire teachings of which are herein incorporated by reference).

A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated herein in their entirety by reference).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The term “biological sample” as used herein includes but is not limited to a biological fluid (for example serum, lymph, blood), cell sample or tissue sample (for example bone marrow or pancreatic biopsy).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

### General Methodology for Producing Antigen-Binding Compounds

The term “antigen-binding compound” refers to a molecule, preferably a proteinaceous molecule, that specifically binds to an antigen, e.g., a BSDL or FAPP polypeptide (or a glycovariant or other variant or derivative thereof as defined herein) with a greater affinity than other compounds do and/or with specificity or selectivity over non-BSDL or FAPP polypeptides. An antigen-binding compound may be a protein, peptide, nucleic acid, carbohydrate, lipid, or small molecular weight compound which binds preferentially to a BSDL or FAPP polypeptide. In a preferred embodiment, the specific binding agent according to the present invention is an antibody, such as a polyclonal antibody, a monoclonal antibody (mAb), a chimeric antibody, a CDR-grafted antibody, a multi-specific antibody, a bi-specific antibody, a catalytic antibody, a humanized antibody, a human antibody, a “naked” antibody, as well as fragments, variants or derivatives thereof, either alone or in combination with other amino acid sequences, provided by known techniques.

Antigen-binding compounds that specifically bind to a BSDL or FAPP polypeptide can be obtained using any suitable method. While their binding to a BSDL or FAPP polypeptide will generally be tested prior to assessing their ability to induce apoptosis or inhibit cell proliferation (e.g. directly killing cells, signalling via apoptotic regulatory pathways, nuclear fragmentation, inhibiting cell growth, inhibiting the cell cycle), it will be appreciated that testing can be carried out in any suitable order, for example as a function of convenience depending on the nature of the assays and antigen-binding compound involved. Compounds of the invention can be identified using any suitable means, for example using high throughput screening to screen large numbers of molecules for BSDL or FAPP binding activity or for pro-apoptotic or anti-cell proliferation activity. Alternatively, smaller numbers or even individual molecules can be prepared and tested, e.g., a small group of compounds related to or derivatives of known compounds having desired properties.

### Testing the Compounds for Activity

Once an antigen-binding compound is obtained it will generally be assessed for its ability to interact with, affect the activity of, and/or induce apoptosis or inhibit the proliferation of target cells. Assessing the antigen-binding compound's ability to induce apoptosis or inhibit the proliferation of target cells can be carried out at any suitable stage of the method, and examples are provided herein. This assessment of the ability to induce apoptosis or inhibit proliferation can be useful at one or more of the various steps involved in the identification, production and/or development of an antibody (or other compound) destined for therapeutic use. For example, pro-apoptotic or anti-cell growth/proliferation activity may be assessed in the context of a screening method to identify candidate antigen-binding compounds, or in methods where an antigen-binding compound is selected and made human suitable (e.g. made chimeric or humanized in the case of an antibody), where a cell expressing the antigen-binding compound (e.g. a host cell expressing a recombinant antigen-binding compound) has been obtained and is assessed for its ability to produce functional antibodies (or other compounds), and/or where a quantity of antigen-binding compound has been produced and is to be assessed for activity (e.g. to test batches or lots of product). Generally the antigen-binding compound will be known to specifically bind to a BSDL or FAPP polypeptide. The step may involve testing a plurality (e.g., a very large number using high throughput screening methods or a smaller number) of antigen-binding compounds for their pro-apoptotic or anti-cell proliferation activity, or testing a single compound (e.g. when a single antibody that binds to a BSDL and/or FAPP polypeptide is provided).

Thus, in addition to binding to a BSDL or FAPP polypeptide, the ability of the antigen-binding compound to induce the apoptosis or inhibit the proliferation of target cells can be assessed. In one embodiment, cells expressing a BSDL and/or FAPP polypeptide are introduced into plates, e.g., 96-well plates, and exposed to various amounts of the relevant compound (e.g. antibodies). By adding a vital dye, i.e. one taken up by intact cells, such as AlamarBlue (BioSource International, Camarillo, Calif.), and washing to remove excess dye, the number of viable cells can be measured by virtue of the optical density (the more cells killed or inhibited by the antibody, the lower the optical density). (See, e.g., Connolly et al. (2001) J Pharm Exp Ther 298:25-33, the disclosure of which is herein incorporated by reference in its entirety). Another example is the use of a stain to detect nuclear fragmentation; DAPI (4′,6-diamidino-2-phenylindole) may be used to bind DNA, and fragmentation can then be visualized by detecting fluoresence. To measure cell proliferation or growth, any suitable method such as determining cell number or density, determining the mitotic index, or any other method to determine the number of cells or their position in the cell cycle can be used. Any other suitable in vitro apoptosis assay, assay to measure cell proliferation or survival, or assay to detect cellular activity can equally be used, as can in vivo assays, e.g. administering the antibodies to animal models, e.g., mice, containing target cells, and detecting the effect of the antibody administration on the survival or activity of the target cells over time.

Assays that can be used to determine whether an antigen-binding compound has pro-apoptotic activity also include assays that determine the compound's effect on components of the cellular apoptotic machinery. For example, as provided in the Examples herein, assays to detect increases or decreases in proteins involved in apoptosis can be used. In one example, a cell (e.g. a SOJ-6 cell or other BDSL and/or FAPP-expressing cell) is exposed to antigen-binding compound, and the level or activity of pro-apoptotic and/or anti-apoptotic proteins is measured, for example Bcl-2 protein family members (e.g. Bcl-2, Bax, Bac, Bad, etc.), or caspases (e.g. caspases 3, 7, 8 and/or 9). A cell which does not express a 16D10 antigen can optionally be used as a control (e.g. PANC-1 cells). Any antigen-binding compound, preferably a human-suitable antibody, that can detectably stop or reverse tumor growth or kill or stop the proliferation of tumor cells, in vitro or in vivo, can be used in the present methods. Preferably, the antigen-binding compound is capable of killing or stopping the proliferation (e.g., preventing an increase in the number of target cells in vitro or in vivo), and most preferably the antigen-binding compound can induce the death of such target cells, leading to a decrease in the total number of such cells. In certain embodiments, the antibody is capable of producing a 10%, 20%, 30%, 40%, 50%, 60%, 70%, 80%, 90%, 95%, 96%, 97%, 98%, 99%, or 100% decrease in the number of target cells or in the proliferation of the target cells. Target cells may be, for example, BDSL or FAPP-expressing cells, cancer cells that express the BDSL or FAPP epitope recognized by 16D10, pancreatic cancer cells, and/or SOJ-6 cells.

In one preferred embodiment, therefore, the present invention provides a method for producing an antigen-binding compound suitable for use in the treatment of a BSDL or FAPP polypeptide-expressing proliferative disorder such as pancreatic cancer, the method comprising the following steps: a) providing a plurality of antigen-binding compounds that specifically bind to a BSDL or FAPP polypeptide; b) testing the ability of the antigen-binding compounds to bind to directly induce apoptosis or inhibit the proliferation of a substantial number of target cells; c) selecting and/or producing an antigen-binding compound from said plurality that is capable of directly inducing apoptosis or inhibiting the proliferation of a target cell. In any of the present methods, a “substantial number” can mean e.g., 30%, 40%, 50%, preferably 60%, 70%, 80%, 90% or a higher percentage of the cells.

Once an antigen-binding compound is obtained it will generally be assessed for its ability to induce ADCC. Testing antibody-dependent cellular cytotoxicity (ADCC) typically involves assessing a cell-mediated cytotoxic reaction in which a FAPP/BSDL-expressing target cell (e.g. a SOJ-6 cell or other BDSL or FAPP-expressing cell) with bound anti-FAPP/BSDL antibody is recognized by an effector cell bearing Fc receptors and is subsequently lysed without requiring the involvement of complement. A cell which does not express a 16D10 antigen can optionally be used as a control (e.g. PANC-1 cells). An exemplary ADCC assay is described in the Examples section herein. Ability to induce ADCC can be tested as the with our without also testing whether the antigen-binding compound has the ability to induce the apoptosis or inhibit the proliferation of target cells. Where an antigen-binding compound is tested for both its ability to (a) induce both ADCC and (b) induce the apoptosis or inhibit the proliferation of target cells, the assays of (a) and (b) can be carried out in any order.

In one preferred embodiment, the present invention provides a method for producing an antigen-binding compound suitable for use in the treatment of a BSDL or FAPP polypeptide-expressing proliferative disorder such as pancreatic cancer, the method comprising the following steps: a) providing a plurality of antigen-binding compounds that specifically bind to a BSDL or FAPP polypeptide; b) testing the ability of the antigen-binding compounds to bind to induce ADCC of a substantial number of target cells; c) selecting and/or producing an antigen-binding compound from said plurality that is capable of directly inducing ADCC of a target cell. In any of the present methods, a “substantial number” can mean e.g., 30%, 40%, 50%, preferably 60%, 70%, 80%, 90% or a higher percentage of the cells.

The antibodies or other compounds of the invention will also typically be assessed not simply with respect to their specificity for BSDL or FAPP antigens, but also their specificity for cancer cells, e.g., pancreatic cancer cells. Standard methods can be used to test the cross-reactivity of the compound or antibody in different cells or tissues, including in vivo methods in animals (e.g., in situ immunostaining) and in vitro methods using isolated cells or cell lines (e.g., western blotting). In a preferred embodiment, the antibodies of the invention do not cross-react with non tumor tissues selected from the group consisting of tonsils, salivary gland, peripheral nerve, lymph node, eye, bone marrow, ovary, oviduct, parathyroid, prostate, spleen, kidney, adrenals, testis, thymus, ureters, uterus, and bladder.

Producing BSDL and/or FAPP Polypeptides

As described herein, in certain embodiments, obtaining antigen-binding compounds (e.g. immunization of a mouse) and/or assessing antigen-binding compounds (e.g. assessing binding to a BSDL or FAPP polypeptide) may involve the use of a BSDL or FAPP polypeptide. BSDL or FAPP polypeptides can be prepared in any suitable manner known in the art. BSDL or FAPP polypeptides and exemplary methods for preparing them are provided, e.g., in WO2005/095594, the entire disclosure of which is incorporated herein by reference. The BSDL or FAPP polypeptide may be a full length BSDL or FAPP polypeptide or a portion thereof. The BSDL or FAPP polypeptides may optionally be joined to another element including but not limited to a second polypeptide, a tag, polymer, or any other suitable molecule. The BSDL or FAPP polypeptides will generally be glycopeptides. In one example, the BSDL or FAPP polypeptide comprises or consists of a glycopeptide comprising or derived from the repeated C-terminal sequences of BSDL, a digestive lipolytic enzyme present in normal pancreatic secretions. In another example, the BSDL or FAPP polypeptide comprises or consists of a glycopeptide comprising or derived from the repeated C-terminal sequences of FAPP (an oncofetal form of BSDL) which is a specific marker of pancreatic pathologies. In certain embodiments, the BSDL or FAPP polypeptide comprises a repeated C-terminal peptide sequences of 11 amino acids, comprising a generally invariant part with 7 amino acids having the sequence Ala Pro Pro Val Pro Pro Thr and a glycosylation site. Said generally invariant part is flanked on either side by a glycine often substituted by a glutamic acid and contains the amino acids Asp and Ser on the N-terminal side. As shown in WO2005/095594, such polypeptides having a glycopeptide structure can be prepared by expression and secretion by a host cell, for example from Chinese hamster ovary (CHO) cells, comprising a gene construct including a DNA molecule coding for one or more repeated sequences of the C-terminal peptide, particularly recombinant of BSDL, for example all or part of the 16 repeated sequences and also comprising a gene construct such as a DNA molecule coding for at least one enzyme with glycosyl-transferase activity, in particular selected in the group consisting of Core 2 β(1-6) N-acetylglucosaminyltransferase, fucosyltransferase FUT3 which has α(1-3) and α(1-4) fucosyltransferase activity, or fucosyltransferase FUT7 which only has α(1-3) fucosyltransferase activity, constituted said specific markers of pancreatic cancer. In one example, WO2005/095594 provides a preferably recombinant, possibly isolated or purified, glycopeptide comprising from 1 to 40 repeated C-terminal polypeptides, composed of 11 amino acids, of BSDL or FAPP, said polypeptides being glycosylated and carrying glycosylated epitopes, optionally giving rise to a specific immunological reaction with induced antibodies in a patient with type I diabetes, and either purified from biological fluids of human or animal origin or recombinant. Recombinant polypeptides can be produced by expression in a conventional host cell comprising an enzymatic machinery necessary for priming a glycosylation, said host cell being genetically modified so as to comprise a gene coding for said polypeptide and a gene coding for one or more enzymes selected from glycosyltransferases and in particular from Corel β(1-6) N-acetylglucosaminyltransferase (abbreviated C2GnT), α(1-3) galactosyltransferase, fucosyltransferase 3 (abbreviated FUT3) and fucosyltransferase 7 (abbreviated FUT7).

### Producing Monoclonal Antibodies Specific for BSDL or FAPP Polypeptides

The present invention involves the production, identification and/or use of antibodies, antibody fragments, or antibody derivatives that are suitable for use in humans and that target a BSDL or FAPP polypeptide. The antibodies of this invention may be produced by any of a variety of techniques known in the art. Typically, they are produced by immunization of a non-human animal, preferably a mouse, with an immunogen comprising a BSDL or FAPP polypeptide. The a BSDL or FAPP polypeptide may comprise entire cells or cell membranes, an isolated BSDL or FAPP polypeptide, or a fragment or derivative of a BSDL or FAPP polypeptide, typically an immunogenic fragment, i.e., a portion of the polypeptide comprising an epitope exposed on the surface of cells expressing the polypeptide. Such fragments typically contain at least 7 consecutive amino acids of the mature polypeptide sequence, even more preferably at least 10 consecutive amino acids thereof. It will be appreciated that any other BSDL or FAPP protein that is sometimes or always present on the surface of all or a fraction of tumor cells, in some or all patients, can be used for the generation of antibodies. In one example, the immunogen is a SOJ-6 cell. In preferred embodiments, the BSDL or FAPP polypeptide used to generate antibodies is a human glycopeptide.

The present antibodies can be full length antibodies or antibody fragments or derivatives. Examples of antibody fragments include Fab, Fab′, Fab′-SH, F(ab′)₂, and Fv fragments; diabodies; single-chain Fv (scFv) molecules; single chain polypeptides containing only one light chain variable domain, or a fragment thereof that contains the three CDRs of the light chain variable domain, without an associated heavy chain moiety; single chain polypeptides containing only one heavy chain variable region, or a fragment thereof containing the three CDRs of the heavy chain variable region, without an associated light chain moiety; and multispecific antibodies formed from antibody fragments. Such fragments and derivatives and methods of preparing them are well known in the art. For example, pepsin can be used to digest an antibody below the disulfide linkages in the hinge region to produce F(ab)′₂, a dimer of Fab which itself is a light chain joined to V_{H}-C_{HI} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology.

In preferred embodiments, the antibodies of the invention are IgG, e.g., IgG1, antibodies, and are tetrameric (bivalent). Such bivalent IgG antibodies are preferred because they are relatively simple to prepare and use, and they combine various properties that allow them to maximally target BSDL/FAPP-expressing tumor cells. In particular, they have sufficient binding affinity (superior to, e.g., monovalent forms; generally having binding affinities at the nanomolar level, e.g., 10-1 nanomolar) to BSDL/FAPP-expressing tumor cells that they can effectively induce apoptosis or inhibit the proliferation of the cells. In addition, as they contain Fc tails, they can effectively induce immune cell mediated killing (ADCC) of the target cells (although it will be appreciated that this feature is not necessary for their efficacy due to the ability to directly target BSDL- or FAPP-expressing cells). Further, bivalent anti-FAPP/BSDL IgG antibodies (in contrast to multimeric, e.g., IgM, forms) are not substantially internalized by target cells, enhancing their ADCC-mediating properties. Finally, as the bivalent anti-BSDL/FAPP antibodies of the invention effectively combine all of these desired features, they can be used “naked,” i.e. without attached moities such as cytotoxic peptides or radioisotopes (although such modified forms, which would introduce yet another mechanism for killing BSDL/FAPP-expressing target cells, can also be used and thus fall within the scope of the present invention).

The preparation of monoclonal or polyclonal antibodies is well known in the art, and any of a large number of available techniques can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to desired polypeptides, e.g., a BSDL or FAPP polypeptide. Also, transgenic mice, or other organisms such as other mammals, may be used to express humanized, chimeric, or similarly modified antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). In one embodiment, the method comprises selecting, from a library or repertoire, a monoclonal antibody or a fragment or derivative thereof that cross reacts with a BSDL or FAPP polypeptide. For example, the repertoire may be any (recombinant) repertoire of antibodies or fragments thereof, optionally displayed by any suitable structure (e.g., phage, bacteria, synthetic complex, etc.).

The step of immunizing a non-human mammal with an antigen may be carried out in any manner well known in the art for (see, for example, E. Harlow and D. Lane, Antibodies: A Laboratory Manual., Cold Spring Harbor Laboratory Press, Cold Spring Harbor, N.Y. (1988)). Generally, the immunogen is suspended or dissolved in a buffer, optionally with an adjuvant, such as complete Freund's adjuvant. Methods for determining the amount of immunogen, types of buffers and amounts of adjuvant are well known to those of skill in the art and are not limiting in any way on the present invention.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be utilized as long as they result in the production of B cells expressing an antibody directed to the antigen used in immunization.

In another embodiment, lymphocytes from a non-immunized non-human mammal are isolated, grown in vitro, and then exposed to the immunogen in cell culture. The lymphocytes are then harvested and the fusion step described below is carried out.

For monoclonal antibodies, which are preferred for the purposes of the present invention, the next step is the isolation of cells, e.g., lymphocytes, splenocytes, or B cells, from the immunized non-human mammal and the subsequent fusion of those splenocytes, or B cells, or lymphocytes, with an immortalized cell in order to form an antibody-producing hybridoma. Accordingly, the term “preparing antibodies from an immunized animal,” as used herein, includes obtaining B-cells/splenocytes/lymphocytes from an immunized animal and using those cells to produce a hybridoma that expresses antibodies, as well as obtaining antibodies directly from the serum of an immunized animal. The isolation of splenocytes, e.g., from a non-human mammal is well-known in the art and, e.g., involves removing the spleen from an anesthetized non-human mammal, cutting it into small pieces and squeezing the splenocytes from the splenic capsule and through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the antibody-producing cells are fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, Calif. U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

The hybridomas can be grown on a feeder layer of macrophages. The macrophages are preferably from littermates of the non-human mammal used to isolate splenocytes and are typically primed with incomplete Freund's adjuvant or the like several days before plating the hybridomas. Fusion methods are described, e.g., in (Goding, “Monoclonal Antibodies: Principles and Practice,” pp. 59-103 (Academic Press, 1986)), the disclosure of which is herein incorporated by reference.

The cells are allowed to grow in the selection media for sufficient time for colony formation and antibody production. This is usually between 7 and 14 days. The hybridoma colonies are then assayed for the production of antibodies that specifically recognize the desired substrate, e.g. a BSDL and/or FAPP polypeptide. The assay is typically a colorimetric ELISA-type assay, although any assay may be employed that can be adapted to the wells that the hybridomas are grown in. Other assays include immunoprecipitation and radioimmunoassay. The wells positive for the desired antibody production are examined to determine if one or more distinct colonies are present. If more than one colony is present, the cells may be re-cloned and grown to ensure that only a single cell has given rise to the colony producing the desired antibody. Positive wells with a single apparent colony are typically recloned and re-assayed to ensure that only one monoclonal antibody is being detected and produced.

Hybridomas or hybridoma colonies can then also be assayed for the production of antibodies capable of inducing apoptosis or inhibiting the cell cycle. This assay can generally be done at any stage of the process so long as an antibody can be obtained and assessed in an in vitro assay. Most preferably, however, once an antibody that specifically recognizes a BSDL and/or FAPP polypeptide is identified, it can be tested for its ability to induce apoptosis or inhibit the growth or proliferation of a cell (e.g. a tumor cell, a SOJ-6 cell, any cell expressing at its surface a BSDL and/or FAPP polypeptide, etc.). Antibodies can also be tested for their ability to induce ADCC (e.g., by virtue of NK cell activation; see Examples).

Hybridomas that are confirmed to be producing a monoclonal antibody of this invention are then grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing the monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Amersham Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

In preferred embodiments, the DNA encoding an antibody that binds a determinant present on a BSDL or FAPP polypeptide is isolated from the hybridoma, placed in an appropriate expression vector for transfection into an appropriate host. The host is then used for the recombinant production of the antibody, variants thereof, active fragments thereof, or humanized or chimeric antibodies comprising the antigen recognition portion of the antibody. Depending on the particular embodiment, the antibodies produced by the host cell can optionally be assessed for their ability to induce apoptosis or inhibit the proliferation of a cell which expresses a BSDL or FAPP polypeptide, or to induce ADCC (e.g., NK cell activation) in the presence of NK cells and (BSDL- or FAPP-expressing) target cells.

DNA encoding the monoclonal antibodies of the invention can be readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant expression in bacteria of DNA encoding the antibody is well known in the art (see, for example, Skerra et al. (1993) Curr. Op. Immunol. 5:256; and Pluckthun (1992) Immunol. Revs. 130:151. Antibodies may also be produced by selection of combinatorial libraries of immunoglobulins, as disclosed for instance in Ward et al. (1989) Nature 341:544.

In a specific embodiment, the antibody binds essentially the same epitope or determinant as the monoclonal antibody 16D10 (see, e.g., WO2005/095594, the entire disclosure of which is herein incorporated by reference). Cells producing the IgM antibody 16D10 were deposited with the Collection Nationale de Culture de Microorganismes (CNCM) in Paris on 16 Mar. 2004 under the number I-3188. In certain embodiments, the antibody is an antibody other than 16D10.

The term “binds to substantially the same epitope or determinant as” the monoclonal antibody x means that an antibody “can compete” with x, where x is 16D10, etc. The identification of one or more antibodies that bind(s) to substantially the same epitope as the monoclonal antibody in question can be readily determined using any one of variety of immunological screening assays in which antibody competition can be assessed. Such assays are routine in the art (see, e.g., U.S. Pat. No. 5,660,827, which is herein incorporated by reference). It will be understood that actually determining the epitope to which the antibody binds is not in any way required to identify an antibody that binds to the same or substantially the same epitope as the monoclonal antibody in question.

For example, where the test antibodies to be examined are obtained from different source animals, or are even of a different Ig isotype, a simple competition assay may be employed in which the control (e.g. 16D10) and test antibodies are admixed (or pre-adsorbed) and applied to a sample containing the epitope-containing protein, e.g. a BSDL or FAPP polypeptide. Protocols based upon ELISAs, radioimmunoassays, western blotting and the use of BIACORE (as described, e.g., in the examples section) are suitable for use in such simple competition studies and are well known in the art.

In certain embodiments, one would pre-mix the control antibodies (e.g. 16D10) with varying amounts (e.g., 1:10 or 1:100) of the test antibodies for a period of time prior to applying to the antigen (e.g. a BSDL or FAPP polypeptide) containing sample. In other embodiments, the control and varying amounts of test antibodies can simply be admixed during exposure to the antigen sample. As long as one can distinguish bound from free antibodies (e.g., by using separation or washing techniques to eliminate unbound antibodies) and the control antibody from the test antibodies (e.g., by using species- or isotype-specific secondary antibodies or by specifically labeling the control antibody with a detectable label) one will be able to determine if the test antibodies reduce the binding of the control antibody to the antigen, indicating that the test antibody recognizes substantially the same epitope as the control. The binding of the (labeled) control antibodies in the absence of a completely irrelevant antibody would be the control high value. The control low value would be obtained by incubating the labeled control antibodies (e.g. 16D10) with unlabeled antibodies of exactly the same type (e.g. 16D10), where competition would occur and reduce binding of the labeled antibodies. In a test assay, a significant reduction in labeled antibody reactivity in the presence of a test antibody is indicative of a test antibody that recognizes the same epitope, i.e., one that “cross-reacts” with the labeled control antibody. Any test antibody that reduces the binding of the labeled control to each the antigen by at least 50% or more, preferably 70%, at any ratio of control:test antibody between about 1:10 and about 1:100 is considered to be an antibody that binds to substantially the same epitope or determinant as the control. Preferably, such test antibody will reduce the binding of the control to the antigen by at least 90%.

In one embodiment, competition can be assessed by a flow cytometry test. Cells bearing a given activating receptor are incubated first with a control antibody that is known to specifically bind to the receptor (e.g., cells expressing a BSDL or FAPP polypeptide, and the 16D10 antibody), and then with the test antibody that has been labeled with, e.g., a fluorochrome or biotin. The test antibody is said to compete with the control if the binding obtained with preincubation with saturating amounts of control antibody is 80%, preferably, 50, 40 or less of the binding (mean of fluorescence) obtained by the antibody without preincubation with the control. Alternatively, a test antibody is said to compete with the control if the binding obtained with a labeled control (by a fluorochrome or biotin) on cells preincubated with saturating amount of antibody to test is 80%, preferably 50%, 40%, or less of the binding obtained without preincubation with the antibody.

In one preferred example, a simple competition assay may be employed in which a test antibody is pre-adsorbed and applied at saturating concentration to a surface onto which is immobilized the substrate for the antibody binding, e.g. a BSDL or FAPP polypeptide, or epitope-containing portion thereof, which is known to be bound by 16D10. The surface is preferably a BIACORE chip. The control antibody (e.g. 16D10) is then brought into contact with the surface at a substrate-saturating concentration and the substrate surface binding of the control antibody is measured. This binding of the control antibody is compared with the binding of the control antibody to the substrate-containing surface in the absence of test antibody. In a test assay, a significant reduction in binding of the substrate-containing surface by the control antibody in the presence of a test antibody is indicative of a test antibody that recognizes the same epitope, i.e., one that “cross-reacts” with the control antibody. Any test antibody that reduces the binding of the control antibody to the antigen-containing substrate by at least 30% or more preferably 40% is considered to be an antibody that binds to substantially the same epitope or determinant as the control antibody. Preferably, such test antibody will reduce the binding of the control antibody to the substrate by at least 50%. It will be appreciated that the order of control and test antibodies can be reversed, that is the control antibody is first bound to the surface and the test antibody is brought into contact with the surface thereafter. Preferably, the antibody having higher affinity for the substrate antigens is bound to the substrate-containing surface first since it will be expected that the decrease in binding seen for the second antibody (assuming the antibodies are cross-reacting) will be of greater magnitude. Further examples of such assays are provided in the Examples and in Saunal et al. (1995) J. Immunol. Meth 183: 33-41, the disclosure of which is incorporated herein by reference.

Once an antibody that specifically recognizes a BSDL or FAPP polypeptide is identified, it can be tested using standard methods for its ability to bind to tumor cells such as the SOJ-6 cell line or any other cell taken from patients with cancer such as pancreatic cancer, and its ability to induce apoptosis or inhibit the proliferation of the same cells. The ability of the cells to activate NK cells or induce ADCC of BSDL- or FAPP-expressing target cells can also be assessed.

### Producing Antibodies Suitable for Use in Humans

Once monoclonal antibodies are obtained, generally in non-human animals, that can specifically bind to a BSDL or FAPP polypeptide, the antibodies will generally be modified so as to make them suitable for therapeutic use in humans. For example, they may be humanized, chimerized, or selected from a library of human antibodies using methods well known in the art. Such human-suitable antibodies can be used directly in the present therapeutic methods, or can be further derivatized. Again, depending on the particular embodiment of the invention, antibodies can be tested for pro-apoptotic or anti-cell proliferation activity before and/or after they are made suitable for therapeutic use in humans.

In one, preferred, embodiment, the DNA of a hybridoma producing an antibody of this invention, e.g. a antibody that binds the same epitope as antibody 16D10, can be modified prior to insertion into an expression vector, for example, by substituting the coding sequence for human heavy- and light-chain constant domains in place of the homologous non-human sequences (e.g., Morrison et al. (1984) PNAS 81:6851), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody of the invention. In one particularly preferred embodiment, the antibody of this invention is humanized. “Humanized” forms of antibodies according to this invention are specific chimeric immunoglobulins, immunoglobulin chains or fragments thereof (such as Fv, Fab, Fab′, F(ab′)₂, or other antigen-binding subsequences of antibodies) which contain minimal sequence derived from the murine or other non-human immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody. In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. For further details see Jones et al. (1986) Nature 321: 522; Reichmann et al. (1988) Nature 332: 323; Verhoeyen et al. (1988) Science 239:1534 (1988); Presta (1992) Curr. Op. Struct. Biol. 2:593; each of which is herein incorporated by reference in its entirety.

The choice of human variable domains, both light and heavy, to be used in making the humanized antibodies is very important to reduce antigenicity. According to the so-called “best-fit” method, the sequence of the variable domain of an antibody of this invention is screened against the entire library of known human variable-domain sequences. The human sequence which is closest to that of the mouse is then accepted as the human framework (FR) for the humanized antibody (Sims et al. (1993) J. Immun., 151:2296; Chothia and Lesk (1987) J. Mol. Biol. 196:901). Another method uses a particular framework from the consensus sequence of all human antibodies of a particular subgroup of light or heavy chains. The same framework can be used for several different humanized antibodies (Carter et al. (1992) PNAS 89:4285; Presta et al. (1993) J. Immunol. 51:1993)).

It is further important that antibodies be humanized while retaining their high affinity for FAPP/BSDL, preferably human FAPP/BSDL, most preferably the epitope specifically recognized by 16D10 (e.g., the antibody can compete for epitope binding with 16D10), and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

Human antibodies may also be produced according to various other techniques, such as by using, for immunization, other transgenic animals that have been engineered to express a human antibody repertoire. In this technique, elements of the human heavy and light chain loci are introduced into mice or other animals with targeted disruptions of the endogenous heavy chain and light chain loci (see, e.g., Jakobovitz et al. (1993) Nature 362:255; Green et al. (1994) Nature Genet. 7:13; Lonberg et al. (1994) Nature 368:856; Taylor et al. (1994) Int. Immun. 6:579, the entire disclosures of which are herein incorporated by reference). Alternatively, human antibodies can be constructed by genetic or chromosomal transfection methods, or through the selection of antibody repertoires using phage display methods. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell (see, e.g., Johnson et al. (1993) Curr Op Struct Biol 3:5564-571; McCafferty et al. (1990) Nature 348:552-553, the entire disclosures of which are herein incorporated by reference). Human antibodies may also be generated by in vitro activated B cells (see, e.g., U.S. Pat. Nos. 5,567,610 and 5,229,275, the disclosures of which are incorporated in their entirety by reference).

In one embodiment, “humanized” monoclonal antibodies are made using an animal such as a XenoMouse® (Abgenix, Fremont, Calif.) for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference. An analogous method can be achieved using a HuMAb-Mouse™ (Medarex).

The antibodies of the present invention may also be derivatized to “chimeric” antibodies (immunoglobulins) in which a portion of the heavy and/or light chain is identical with or homologous to corresponding sequences in the original antibody, while the remainder of the chain(s) is identical with or homologous to corresponding sequences in antibodies derived from another species or belonging to another antibody class or subclass, as well as fragments of such antibodies, so long as they exhibit the desired biological activity (see, e.g., Morrison et al. (1984) PNAS 81:6851; U.S. Pat. No. 4,816,567).

### Structural Properties of Recombinant 16D10 Antibodies

In one preferred embodiment, the antibody of the invention is a chimeric or humanized IgG antibody prepared using the variable domain sequences (e.g. the entire variable domain, a portion thereof, or CDRs) of the 16D10 antibody (or another antibody that binds to the same epitope as 16D10). For example, the antibody can be Rec 16d10 or an equivalent antibody, a chimeric antibody in which the Cu2, Cu3, and Cu4 domains of the mouse heavy chain constant region of 16D10 have been replaced by a human IgG1 Fc. In another preferred embodiment, the antibody is a chimeric antibody in which the VH and VL of an anti-FAPP/BSDL antibody such as 16D10 are replaced by human IgG (e.g. IgG1) constant regions for both heavy and light chains.

Preferred antibodies of the invention are the bivalent monoclonal antibodies comprising the variable region or CDRs of 16D10 as produced, isolated, and structurally and functionally characterized and described herein. In one example the antibody is the chimeric antibody described in Example 9 (rec16D10); in another example, the antibody is the alternative bivalent chimeric antibody made of the (two) heavy chain(s) comprising the heavy chain variable region of 16D10 fused to a human IgG1 constant region and the (two) light chain(s) comprising the light chain variable region of 16D10 fused to a human IgL Kappa constant region. Full-length, variable, and CDR sequences of these antibodies are set forth in Table 1.

TABLE 1

| Antibody portion | SEQ ID NO: |
|---|---|
| VH-16D10-HuIgG1 (from rec16D10 | 3 |
| of Example 9) | |
| Rec16D10 L chain (from rec16D10 | 4 |
| of Example 9) | |
| VH-16D10-HuIgG1 (alternative | 5 |
| 16D10 antibody) | |
| VL16D10-HuIgL Kappa (alternative | 6 |
| 16D10 antibody) | |
| 16D10 VH region | 7 |
| 16D10 VL region | 8 |
| 16D10 VH CDR1 | 9 |
| 16D10 VH CDR2 | 10 |
| 16D10 VH CDR3 | 11 |
| 16D10 VL CDR1 | 12 |
| 16D10 VL CDR2 | 13 |
| 16D10 VL CDR3 | 14 |

Accordingly, in one aspect, the invention provides an isolated monoclonal antibody, or antigen binding portion thereof, comprising: (a) a VH region comprising an amino acid sequence selected from the group consisting of SEQ ID NOs: 3, 5, 7 and 9-11, and (b) a VL region comprising an amino acid sequence selected from the group consisting of SEQ ID NOs: 4, 6, 8 and 12-14; wherein the antibody specifically binds a BSDL or FAPP polypeptide. Preferred heavy and light chain combinations include: (a) a heavy chain comprising the amino acid sequence of SEQ ID NO: 3; and (b) a light chain comprising the amino acid sequence of SEQ ID NO: 4; (a) a heavy chain comprising the amino acid sequence of SEQ ID NO: 5; and (b) a light chain comprising the amino acid sequence of SEQ ID NO: 6; and (a) a heavy chain variable region comprising the amino acid sequence of SEQ ID NO: 7; and (b) a light chain variable region comprising the amino acid sequence of SEQ ID NO: 8.

In another aspect, the invention provides antibodies that comprise the heavy chain and light chain CDR1s, CDR2s and/or CDR3s of 16D10, or combinations thereof. The CDR regions are delineated using the Kabat system (Kabat et al. (1991) Sequences of Proteins of Immunological Interest, Fifth Edition, U.S. Department of Health and Human Services, NIH Publication No. 91-3242). The heavy chain CDRs of 16D10 are located at amino acids positions 31-35 (CDR1; Chotia numbering is 26-35 for CDR1), positions 50-67 (CDR2) and positions 97-106 (CDR3) in SEQ ID NO: 7. The light chain CDRs of 16D10 are located at amino acids positions 24-40 (CDR1), positions 56-62 (CDR2) and positions 95-102 (CDR3) in SEQ ID NO: 8.

Accordingly, in another aspect, the invention provides an isolated monoclonal antibody, or antigen binding portion thereof comprising: (a) a VH CDR1 comprising an amino acid sequence of SEQ ID NO: 9; (b) a VH CDR2 comprising an amino acid sequence of SEQ ID NO: 10; (c) a VH CDR3 comprising an amino acid sequence of SEQ ID NO: 11; (d) a VL CDR1 comprising an amino acid sequence of SEQ ID NO: 12; (e) a VL CDR2 comprising an amino acid sequence of SEQ ID NO:13; and (f) a VL CDR3 comprising an amino acid sequence of SEQ ID NO: 14; wherein the antibody specifically binds FAPP or BSDL. Preferably said antibody comprises a heavy chain variable region comprising VH CDR1, VH CDR2 and VH CDR3 fused to a human IgG chain constant region, and a light chain variable region comprising VL CDR1, VH CDR2 and VH CDR3 fused to human kappa chain constant region. Preferably said human IgG chain constant region comprises the amino acid sequence of SEQ ID NO 15, or a portion thereof, or a sequence at least 80%, 90% or 95% identical thereto. Preferably said human kappa chain constant region comprises the amino acid sequence of SEQ ID NO 16, or a portion thereof, or a sequence at least 80%, 90% or 95% identical thereto. Preferably the antibody is a tetramer comprising two of said heavy chains and two of said light chains.

In certain embodiments, an antibody of the invention comprises a VH region from a VH J558.48 murine germline H chain immunoglobulin gene and/or a VL region from a VK 8-27 murine germline L chain immunoglobulin gene.

In one aspect, the invention provides an isolated monoclonal antibody, or antigen binding portion thereof, comprising: (a) a VH region described herein (e.g. a variable region, portion thereof, or a variable region comprising VH CDR1, CDR2 and/or CDR3 described herein) fused to a human IgG chain constant region, and (b) a VL region described herein (i.e. a variable region, portion thereof, or a variable region comprising VH CDR1, CDR2 and/or CDR3 described herein) fused to human kappa chain constant region; wherein the antibody specifically binds a BSDL or FAPP polypeptide. Exemplary IgG chain constant regions include a constant region having the sequence of SEQ ID NO: 15 obtained from the antibody rituximab (Rituxan™, Genentech, Calif.), or a portion thereof. Exemplary to human kappa chain constant regions include a constant region having the sequence of SEQ ID NO: 16 obtained from the antibody rituximab (Rituxan™, Genentech, Calif.), or a portion thereof.

In yet another embodiment, an antibody of the invention comprises heavy and light chain variable regions comprising amino acid sequences that are homologous to the amino acid sequences of the preferred antibodies described herein, and wherein the antibodies retain the desired functional properties of the anti-FAPP/BSDL antibodies of the invention. For example, the invention provides an isolated monoclonal antibody, or antigen binding portion thereof, comprising a heavy chain variable region and a light chain variable region, wherein: (a) the VH region comprises an amino acid sequence that is at least 80% identical to an amino acid sequence selected from the group consisting of SEQ ID NOs: 3, 5, 7 and 9-11; (b) the VL region comprises an amino acid sequence that is at least 80% identical to an amino acid sequence selected from the group consisting of SEQ ID NOs: 4, 6, 8 and 12-14; (c) the antibody specifically binds to a FAPP or BSDL polypeptide and exhibits at least one of the functional properties described herein, preferably several of the functional properties described herein.

In other embodiments, the CDR, VH and/or VL, or constant region amino acid sequences may be 85%, 90%, 95%, 96%, 97%, 98% or 99% identical to the sequences set forth above. An antibody having CDR, VH and/or VL regions having high (i.e., 80% or greater) identity to the CDR, VH and/or VL, or constant region regions of the sequences set forth above, can be obtained by mutagenesis (e.g., site-directed or PCR-mediated mutagenesis) of nucleic acid molecules encoding the CDR, VH and/or VL of SEQ ID NOs: 3 to 14, or the constant regions of SEQ ID NOs: 15 and 16, followed by testing of the encoded altered antibody for retained function (e.g., FAPP/BSDL binding affinity, induction of apoptosis or slowing proliferation of tumor cells, induction of ADCC).

The percent identity between the two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm in a sequence analysis software. Protein analysis software matches similar sequences using measures of similarity assigned to various substitutions, deletions and other modifications, including conservative amino acid substitutions.

The percent identity between two amino acid sequences can be determined, e.g., using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Polypeptide sequences can also be compared using FASTA, applying default or recommended parameters. A program in GCG Version 6.1., FASTA (e.g., FASTA2 and FASTA3) provides alignments and percent sequence identity of the regions of the best overlap between the query and search sequences (Pearson, Methods Enzymol. 1990; 183:63-98; Pearson, Methods Mol. Biol. 2000; 132:185-219).

The percent identity between two amino acid sequences can also be determined using the algorithm of E. Meyers and W. Miller (Comput. Appl. Biosci., 1988; 11-17) which has been incorporated into the ALIGN program (version 2.0), using a PAM120 weight residue table, a gap length penalty of 12 and a gap penalty of 4.

Another algorithm for comparing a sequence to a other sequences contained in a database is the computer program BLAST, especially blastp, using default parameters. See, e.g., Altschul et al., J. Mol. Biol. 1990; 215:403-410; Altschul et al., Nucleic Acids Res. 1997; 25:3389-402 (1997); each herein incorporated by reference. The protein sequences of the present invention can there be used as a “query sequence” to perform a search against public databases to, for example, identify related sequences. Such searches can be performed using the XBLAST program (version 2.0) of Altschul, et al. 1990 (supra). BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., 1997 (supra). When utilizing BLAST and Gapped BLAST programs, the default parameters of the respective programs (e.g., XBLAST and NBLAST) can be used. See http://www. ncbi.nlm.nih.gov.

In certain embodiments, an antibody of the invention comprises a VH region comprising CDR1, CDR2 and CDR3 sequences and a VL region comprising CDR1, CDR2 and CDR3 sequences, wherein one or more of these CDR or variable region sequences comprise specified amino acid sequences based on the preferred antibodies described herein (e.g. 16D10 and any of SEQ ID NOs 3-14), or conservative modifications thereof, and wherein the antibodies retain the desired functional properties of the anti-FAPP/BSDL antibodies of the invention. Conservative sequence modifications can be any amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. “Conservative” amino acid substitutions are typically those in which an amino acid residue is replaced with an amino acid residue having a side chain with similar physicochemical properties. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine).

Thus, one or more amino acid residues within the CDR regions of an antibody of the invention can be replaced with other amino acid residues from the same side chain family and the altered antibody can be tested for retained function (i.e., the functions set forth in (c), (d) and (e) above) using the functional assays described herein.

The nucleic acid sequences encoding the 16D10 antibody heavy chain and light chain variable regions are shown in SEQ ID NOS 1 and 2, respectively. In one embodiment the invention provides a bivalent monoclonal antibody that comprises the variable heavy chain region of 16D10 transcribed and translated from a nucleotide sequence comprising SEQ ID NO 1 or a fragment thereof (e.g. a sequence encoding CDR1, CDR2 and/or CDR3 of 16D10 VH region), and the variable light chain region of 16D10 transcribed and translated from a nucleotide sequence comprising SEQ ID NO 2 or a fragment thereof (e.g. a sequence encoding CDR1, CDR2 and/or CDR3 of the 16D10 VL region). In yet another preferred embodiment, a bivalent antibody comprises in its heavy chain(s) a CDR1, CDR2 and/or CDR3 or heavy chain variable region present in the antibody 16D10 deposited with the Collection Nationale de Culture de Microorganismes (CNCM) in Paris on 16 Mar. 2004 under the number I-3188, and in its light chain(s) a CDR1, CDR2 and/or CDR3 or light chain variable region present in said antibody 16D10 deposited with the Collection Nationale de Culture de Microorganismes (CNCM) in Paris on 16 Mar. 2004 under the number I-3188.

### Constant Region Optimization

In view of the ability of the antibodies of the invention to induce ADCC of cells expressing FAPP or BSDL polypeptides, the antibodies of the invention can also be made with modifications that increase their ability to induce ADCC. Typical modifications include modified human IgG1 constant regions comprising at least one amino acid modification (e.g. substitution, deletions, insertions), and/or altered types of glycosylation, e.g., hypofucosylation. Such modifications can for example increase binding to FcyRIIIa on effector (e.g. NK) cells.

Certain altered glycosylation patterns in constant regions have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 06/133148; WO 03/035835; WO 99/54342 80, each of which is incorporated herein by reference in its entirety.

Generally, such antibodies with altered glycosylation have a particular N-glycan structure that produces certain desirable properties, including but not limited to, enhanced ADCC and effector cell receptor binding activity when compared to non-modified antibodies or antibodies having a naturally occurring constant region and produced by murine myeloma NSO and Chinese Hamster Ovary (CHO) cells (Chu and Robinson, Current Opinion Biotechnol. 2001, 12: 180-7), HEK293T-expressed antibodies as produced herein in the Examples section, or other mammalian host cell lines commonly used to produce recombinant therapeutic antibodies.

Monoclonal antibodies produced in mammalian host cells contain an N-linked glycosylation site at Asn297 of each heavy chain. Glycans on antibodies are typically complex biatennary structures with very low or no bisecting N-acetylglucosamine (bisecting GlcNAc) and high levels of core fucosylation. Glycan temini contain very low or no terminal sialic acid and variable amounts of galactose. For a review of glycosylation on antibody function, see, e.g., Wright & Morrison, Trend Biotechnol. 15:26-31 (1997). Considerable work shows that changes to the sugar composition of the antibody glycan structure can alter Fc effector functions. The important carbohydrate structures contributing to antibody activity are believed to be the fucose residues attached via alpha-1,6 linkage to the innermost N-acetylglucosamine (GlacNAc) residues of the Fc region N-linked oligosaccharides (Shields et al., 2002). FcγR binding requires the presence of oligosaccharides covalently attached at the conserved Asn297 in the Fc region. Non-fucosylated structures have recently been associated with dramatically increased in vitro ADCC activity.

Historically, antibodies produced in CHO cells contain about 2 to 6% in the population that are nonfucosylated. YB2/0 (rat myeloma) and Lec13 cell line (a lectin mutant of CHO line which has a deficient GDP—mannose 4,6-dehydratase leading to the deficiency of GDP-fucose or GDP sugar intermediates that are the substrate of alpha6-fucosyltransferase have been reported to produce antibodies with 78 to 98% non-fucosylated species. In other examples, RNA interference (RNAi) or knock-out techniques can be employed to engineer cells to either decrease the FUT8 mRNA transcript levels or knock out gene expression entirely, and such antibodies have been reported to contain up to 70% non-fucosylated glycan. In other examples, a cell line producing an antibody can be treated with a glycosylation inhibitor; Zhou et al. Biotech. and Bioengin. 99: 652-665 (2008) described treatment of CHO cells with the alpha-mannosidase I inhibitor, kifunensine, resulting in the production of antibodies with non-fucosylated oligomannose-type N-glucans.

Thus, in one embodiment of the invention, an antibody will comprise a constant region comprising at least one amino acid alteration in the Fc region that improves antibody binding to FcyRIIIa and/or ADCC. In another aspect, an antibody composition of the invention comprises a chimeric, human or humanized antibody described herein, wherein at least 20, 30, 40, 50, 60, 75, 85 or 95% of the antibodies in the composition have a constant region comprising a core carbohydrate structure which lacks fucose.

While antibodies in underivatized or unmodified form, particularly of the IgG1 or IgG3 type, or underivatived antibodies comprising a modification in the constant region to improve antibody binding to FcyRIIIa and/or ADCC, are expected to induce the apoptosis of and/or inhibit the proliferation of FAPP or BDSL polypeptide-expressing tumor cells such as in those from a pancreatic cancer patient, it is also possible to prepare derivatized antibodies to make them cytotoxic. When bivalent IgG forms of such derivatived antibodies are used, they can thus target tumor cells in at least three distinct ways: by ADCC (e.g. when the antibodies comprise bind Fc receptors, for example via their constant regions), by inducing apoptosis or inhibiting cell proliferation, and by killing the cell via the cytotoxic moiety. In one embodiment, once the antibodies are isolated and rendered suitable for use in humans, they are derivatized to make them toxic to cells. In this way, administration of the antibody to cancer patients will lead to the relatively specific binding of the antibody to FAPP and/or BDSL polypeptide-expressing cancer cells, thereby providing an additional means for directly killing or inhibiting the cells.

### Use of Compounds in Therapy

The antibodies produced using the present methods are particularly effective at treating pancreatic cancer and/or tumors which express BDSL or FAPP polypeptides (e.g. breast cancers).

In one aspect, when practicing the invention, the cancer in patients can be characterized or assessed. This can be useful to determine whether a cancer can advantageously be treated according to the invention. For example, since the antigen-binding compounds of the invention have pro-apoptotic and anti-cell proliferation activity, they may be used to directly kill tumor cells and/or reduce or limit the volume of a tumor. The antigen-binding compounds may have particular advantageous properties in the treatment of BDSL or FAPP polypeptide-expressing tumors having spread beyond in situ carcinoma, having a size of less than 2 cm in any direction, and/or in the treatment of metastases and/or metastatic tumors.

The compounds of the invention are well adapted to treat pancreatic cancer where it is useful and/or necessary to induce the death of the tumor cells or slow their growth or proliferation. This includes but is not limited to: a pancreatic cancer where the tumor is established or has spread, where the cancer has progressed beyond in situ carcinoma, for example where the pancreatic cancer is classified as at least a Stage I cancer and/or where the size of the tumor in the pancreas is 2 cm or less in any direction, or where the pancreatic cancer is classified as at least a Stage 2 cancer and/or where the size of the tumor in the pancreas is more than 2 cm in any direction, where the pancreatic cancer is classified as a Stage 2 cancer and/or the cancer has started to grow into nearby tissues around the pancreas, but not inside the nearby lymph nodes, where the pancreatic cancer is classified as a Stage 3 cancer and/or may have grown into the tissues surrounding the pancreas, or where the pancreatic cancer is classified as a Stage 4 cancer and/or has grown into nearby organs. The ability to kill or inhibit the growth of tumor cells in tumors that have progressed beyond in situ carcinoma is significant in pancreatic cancers since such cancers are often diagnosed at advanced stage of development.

Any one or more of commonly practiced methods are be used to assess or characterize a pancreatic cancer. Pancreatic cancer is usually diagnosed with tests and procedures that produce pictures of the pancreas and the area around it. The process used to find out if cancer cells have spread within and around the pancreas is called staging. Tests and procedures to detect, diagnose, and stage pancreatic cancer are usually done at the same time. Stage of the disease and whether or not the pancreatic cancer can be removed by surgery can be assessed by procedures such as chest x-ray, physical exam and history, CT scan (CAT scan), MRI (magnetic resonance imaging), PET scan (positron emission tomography scan), endoscopic ultrasound (EUS), laparoscopy, endoscopic retrograde cholangiopancreatography (ERCP), percutaneous transhepatic cholangiography (PTC), and/or by biopsy. In biopsy, cells or tissues are removed so they can be viewed under a microscope by a pathologist to check for signs of cancer, and/or optionally for expression of BDSL or FAPP polypeptides.

As discussed herein, the inventors have demonstrated using SDS-PAGE and western blotting that treatment of cells with 16D10 induces a decrease of the anti-apoptotic protein Bcl-2 as well as an increase of pro-apoptotic Bax protein. It has also been demonstrated that the antibody increases p53 and GSK-313 activity and lowers cyclin D1 levels. The antigen-binding compounds and methods of the invention can therefore be advantageously used in a method of regulating Bcl-2 family member protein activity in a cell, preferably regulating Bcl-2 family member protein levels in a cell, preferably decreasing Bcl-2 protein expression and/or increasing Bax protein expression. Similarly, the antigen-binding compounds and methods of the invention can also be advantageously used in a method of regulating cell cycle activity in a cell and/or blocking cells at the G1/S transition, preferably increasing p53 or GSK-313 activity or and/or decreasing cyclin D1 levels. The cell may be any cell that expresses a BDSL or FAPP polypeptide, preferably a tumor cell (e.g., pancreatic tumor cell), preferably a cell that expresses a BDSL or FAPP polypeptide in a lipid raft. The cell may be a cell (e.g. tumor cell) in which one or more Bcl-2 family members' activity (or p53, cyclin D1, or GSK-3β) (e.g. biological activity and/or protein expression) is dysregulated, that is, activity is increased or decreased compared to a normal cell (e.g. non-tumor cell), and/or characterized by an imbalance with respect to other pro- or anti-apoptotic or pro- or anti-cell cycle proteins.

The members of the human Bcl-2 family share one or more of the four characteristic domains of homology entitled the Bcl-2 homology (BH) domains (named BH1, BH2, BH3 and BH4). The BH domains are known to be crucial for function, and deletion of these domains via molecular cloning affects survival/apoptosis rates. The anti-apoptotic Bcl-2 proteins, such as Bcl-2 and Bcl-xL, conserve all four BH domains. The BH domains also serve to subdivide the pro-apoptotic Bcl-2 proteins into those with several BH domains (e.g. Bax, Bcl-xS and Bak) or those proteins that have only the BH3 domain (e.g. Bid, Bim and Bad).

Bcl-2 is essential to the process of apoptosis because it suppresses the initiation of the cell-death process. Immunohistochemical staining has typically been used to detect levels of Bcl-2 family members' expression in tumors. It has been found that in some cases pancreatic tumors may overexpress Bcl-2; these tumor cells are expected to be resistant to apoptosis. It has also been shown that about 50% of pancreatic tumors overexpress the anti-apoptotic Bcl-xL, and that enhanced expression of Bcl-xL is related to a shorter patient survival, whereas the upregulation of Bax is associated with longer survival.

In one aspect, the invention provides a method of treating or killing a BSDL or FAPP polypeptide-expressing cell having a Bcl-2 family member dysregulation, comprising bringing the cell into contact with an antigen-binding compound of the invention. In another aspect, the invention provides a method of treating a patient having a tumor having a Bcl-2 family member dysregulation, comprising administering to the patient a pharmaceutically effective amount of an antigen-binding compound of the invention.

In one aspect, the invention provides a method of treating or killing a BSDL or FAPP polypeptide-expressing cell, comprising (a) determining whether the cell is characterized by a Bcl-2 family member dysregulation, and (b) if the cell is characterized by a Bcl-2 family member dysregulation, bringing the cell into contact with an antigen-binding compound of the invention. In another aspect, the invention provides a method of treating a patient having a tumor, comprising (a) determining whether a patient has a tumor characterized by a Bcl-2 family member dysregulation, and (b) if the tumor is characterized by a Bcl-2 family member dysregulation, administering to the patient a pharmaceutically effective amount of an antigen-binding compound of the invention.

In another embodiment, the invention provides a method of treating or killing a BSDL- or FAPP-expressing cell, comprising a) determining if the cell is characterized by overexpression of cyclin D1 or lack of p53 or GSK-3β activity, and b) if the cell is characterized by overexpression of cyclin D1 or lack of p53 or GSK-3β activity, bringing the cell into contact with an antigen-binding compound of the invention. In one method, the cell is a tumor cell present in a patient with cancer, e.g., pancreatic cancer, and the method is used to treat the patient.

Determining whether a tumor or cell has a Bcl-2 family member dysregulation (or altered cyclin D1 or p53 or GSK-3β activity or levels) can be carried out by any suitable method, for example immunohistochemistry or nucleic acid probe or primer based approaches, and may detect any of a number of parameters, such as for example determining whether the tumor or cell harbors a mutation capable of giving rise to a Bcl-2 family member dysregulation (or altered cyclin D1 or p53 or GSK-3β activity or levels), a mutated Bcl-2 family member (or cyclin D1 or p53 or GSK-3β), increased or decreased expression of a Bcl-2 family member (or cyclin D1 or p53 or GSK-3β) (e.g. by determining protein level and/or transcripts). In one aspect, a dysregulation comprises an increased activity of an anti-apoptotic Bcl-2 family member (e.g. Bcl-2, Bcl-xL) and/or a decreased activity of a pro-apoptotic Bcl-2 family member (e.g. Bax, etc.).

As summarized in Giovannetti et al. (2006) Mol. Cancer. Ther. 5(6): 1387-1395, it is thought that the modulation of apoptotic pathways might be one of the reasons why pancreatic cancer shows only limited sensitivity to anticancer chemotherapy treatment. Fahy et al. (British Journal of Cancer (2003) 89, 391-397) investigated the regulation of Bcl-2 and Bax in chemosensitization. Activation of the serine/threonine kinase AKT is common in pancreatic cancer; inhibition of which sensitizes cells to the apoptotic effect of chemotherapy. Fahy et al. examined activation of the NF-kB transcription factor and subsequent transcriptional regulation of BCL-2 gene family in pancreatic cancer cells. Inhibition of either phosphatidylinositol-3 kinase or AKT led to a decreased protein level of Bcl-2 and an increased protein level of Bax. Furthermore, inhibition of AKT decreased the function of NF-kB, which is capable of transcriptional regulation of the Bcl-2 gene. Inhibiting this pathway had little effect on the basal level of apoptosis in pancreatic cancer cells, but increased the apoptotic effect of chemotherapy.

The antigen-binding compounds of the invention can therefore be advantageously used to sensitize a BDSL or FAPP polypeptide-expressing cell, particularly a tumor cell (e.g., pancreatic tumor cell), to treatment with a chemotherapeutic agent. The agent may generally be any agent that requires a cell to be able to undergo apoptosis in order to be effective. In a preferred embodiment, the agent is an agent to which pancreatic tumors or tumor cells are known to be or to become partly or completely resistant. In one embodiment, the antigen-binding compounds of the invention can be used to treat a patient having a chemotherapy resistant, BDSL or FAPP polypeptide-expressing tumor. In another embodiment, the antigen-binding compounds of the invention can be used to treat a patient having a BDSL and/or FAPP polypeptide-expressing tumor, in combination with a chemotherapeutic agent, generally an agent which requires as part of its mechanism of action, that its cellular target be able to undergo apoptosis (or not be resistant to apoptosis). Optionally, the tumor or patient has been previously treated with a chemotherapeutic agent and/or the tumor is resistant to treatment with a chemotherapeutic agent (i.e. in the absence of conjoint treatment with an antigen-binding compound of the invention). In one example, particularly for the treatment of pancreatic cancer, the agent is a nucleoside analog (e.g. gemcitabine). In another example, the agent is a taxane (e.g. paclitaxel and docetaxel and analogs thereof, etc.). In another example, the agent is an antimetabolite, an alkylating agent, a cytotoxic antibiotic or a topoisomerase inhibitor. Although it will be appreciated that the antigen-binding compound of the invention and chemotherapeutic agent will often be administered separately, also encompassed is a composition comprising an antigen-binding compound of the invention and a chemotherapeutic agent. Such composition can be used in any of the methods described herein.

The invention also provides compositions, e.g., pharmaceutical compositions, that comprise any of the present compounds, antibodies, including fragments and derivatives thereof, in any suitable vehicle in an amount effective to inhibit the proliferation or activity of, or to kill, cells expressing a BSDL or FAPP polypeptide in patients. The composition generally further comprises a pharmaceutically acceptable carrier. It will be appreciated that the present methods of administering antibodies and compositions to patients can also be used to treat animals, or to test the efficacy of any of the herein-described methods or compositions in animal models for human diseases.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to another embodiment, the antibody compositions of this invention may further comprise one or more additional therapeutic agents, including agents normally utilized for the particular therapeutic purpose for which the antibody is being administered (e.g. pancreatic cancer). The additional therapeutic agent will normally be present in the composition in amounts typically used for that agent in a monotherapy for the particular disease or condition being treated.

In connection with solid tumor treatment, the present invention may be used in combination with classical approaches, such as surgery, radiotherapy, chemotherapy, and the like. The invention therefore provides combined therapies in which compounds which bind a BSDL or FAPP polypeptide are used simultaneously with, before, or after surgery or radiation treatment; or are administered to patients with, before, or after conventional chemotherapeutic, radiotherapeutic or anti-angiogenic agents, or targeted immunotoxins. The compounds which bind a BSDL or FAPP polypeptide and anti-cancer agents may be administered to the patient simultaneously, either in a single composition, or as two distinct compositions using different administration routes.

When one or more agents (e.g., anti-cancer agent) are used in combination with the present therapy, there is no requirement for the combined results to be additive of the effects observed when each treatment is conducted separately. Although at least additive effects are generally desirable, any increased tumor cell proliferation effect above one of the single therapies would be of benefit. Also, there is no particular requirement for the combined treatment to exhibit synergistic effects, although this is certainly possible and advantageous. The treatment with a compound which bind a BSDL or FAPP polypeptide may precede, or follow, the other anti-pancreatic cancer agent treatment by, e.g., intervals ranging from minutes to weeks and months.

Since the compounds which bind a BSDL or FAPP polypeptide of the present invention induce apoptosis or inhibit cell proliferation directly on cells expressing BSDL or FAPP polypeptide rather than depending mainly on an immune mediated mechanism (e.g. ADCC), it is expected that the compounds of the invention can be used in conjunction with agents that have been reported to have a negative or inhibitory effect on the immune system. For example, chemotherapy may be used to treat cancers, including pancreatic cancer. A variety of chemotherapeutic agents may be used in the combined treatment methods disclosed herein. Chemotherapeutic agents such as tyrosine kinase inhibitors have been reported to have adverse effects on patients' immune response in vivo and metalloproteinase inhibitors have been reported to have hematologic toxicity. Further, the compounds can be effectively used in immunocompromised patients, such as patients with AIDS or other immune diseases (e.g., lymphomas, leukemia), or in patients taking immunosuppressive drugs such as cyclosporins, azathioprines (Imuran), or corticosteroids in conjunction with organ transplantation or as treatment for immune disorders such as psoriasis, rheumatoid arthritis, or Crohn's disease.

Chemotherapeutic agents suitable for use in combination with the antigen-binding compounds which bind a BSDL or FAPP polypeptide for the treatment or prevention of disease (e.g. pancreatic cancer) include, for example, cytotoxic antibiotics, agents that interfere with DNA replication (such as nucleoside analogues, i.e. gemcitabine), mitosis and chromosomal segregation, and agents that disrupt the synthesis and fidelity of polynucleotide precursors.

Exemplary suitable chemotherapeutic agents include:
alkylating agents (e.g. cyclophosphamide, fosfamide, melphalan, mitomycine C) including also platinum-based chemotherapy drugs such as cisplatin (Cisplatyl™), paraplatin (Carboplatin™), oxaliplatin (Eloxatine™), satraplatin, nedaplatin (Aqupla), triplatin tetranitrate;
antimetabolites (e.g. purine analogues such as fludarabine, thioguanine, mercaptopurine, azathioprine, pyrimidine analogues such as 5-fluorouracil, cytarabine, gemcitabine, floxuridine, antifolates such as methotrexate, pemetrexed, raltitrexed, nitrosoureas);
taxanes (e.g. docetaxel, larotaxel, ortataxel, paclitaxel, tesetaxel);
Epothilones (e.g. ixabepilone);
vinca alkaloids (e.g. vinblastine, vincristine, vinflunine, vindesine, vinorelbine);
tyrosine kinase inhibitors (e.g. erlotinib, sorafenib, sunitinib);
topoisomerase inhibitors (e.g. topoisomerase I or II inhibitors, anthracyclines such as aclarubicin, daunorubicin, doxorubicin, epirubicin, idarubicin, amrubicin, pirarubicin, valrubicin, zorubicin, anthracenediones such as mitoxantrone, pixantrone, podophyllum-derived compounds such as etoposide, teniposide, camptotheca based agents such as camptothecin, topotecan, irinotecan, rubitecan, belotecan);
metalloproteinase inhibitors; and
COX-2 inhibitors.

In one embodiment, the chemotherapeutic agent is a tyrosine kinase inhibitor. Tyrosine kinase inhibitors are able to antagonize numerous kinds of cellular receptors. For example, some of the receptors that tyrosine kinase inhibitors are able to antagonize, include, but are not limited to, platelet-derived growth factor receptors (PDGFRα and PDGFRβ), vascular endothelial growth factor receptors (VEGFR1, VEGFR2 and VEGFR3), epidermal growth factor receptor (EGFR), stem cell factor receptor (KIT), Fms-like tyrosine kinase-3 (FLT3), colony stimulating factor receptor Type 1 (CSF-IR), Raf kinase, the Src family of kinases, and the glial cell-line derived neurotrophic factor receptor (RET). Examples of small molecule organic compounds that inhibit tyrosine kinases are: bis-monocylic, bicyclic and heterocyclic aryl compounds, vinyleneazaindole derivatives, 1-cyclopropyl-4-pyridylquinolones, styryl compounds, styryl-substituted pyridyl compounds, quinazoline derivatives, selenaindoles and selenides, tricyclic polyhydroxylic compounds, benzylphosphonic acid compounds, and pyrrole substituted 2-indolinones. These compounds, their preparation and use are disclosed in, among other references, International Appl. Publ. Nos. WO 92/20642, WO 94/14808, WO 94/03427, WO 92/21660, WO 91/15495; U.S. Pat. Nos. 5,330,992, 5,217,999, 5,302,606, 6,573,293, 7,125,905; and European Pat. Appl. Publ. No. EP 0 566 266; each of which is incorporated herein by reference in its entirety. Additional examples of small molecule organic compounds that inhibit tyrosine kinases are: sorafenib, which is known commercially as Nexavar®; dasatinib, which is known commercially as Sprycel®; erlotinib, which is known commercially as Tarceva®; gefitinib, which is known commercially as Iressa®; imatinib, which is known commercially as Gleevec®; lapatinib, which is known commercially as Tykerb®; nilotinib; sunitinib, which is known commercially as Sutent®; and vandetanib, which is known commercially as Zactima®, and masatinib (AB Science).

In one embodiment, the chemotherapeutic agent is a platinum-containing drug, e.g. cisplatin. Cisplatin (cis-diaminedichloridoplatinum(II) (CDDP, (SP-4-2)-diaminedichloridoplatinum) is a platinum-based chemotherapy drug used to treat various types of cancers, including sarcomas, some carcinomas (e.g. small cell lung cancer, and ovarian cancer), lymphomas and germ cell tumors. It is the member of a class including carboplatin and oxaliplatin. Platinum complexes are formed in cells, which bind and cause Crosslinking of DNA—ultimately triggering apoptosis, or programmed cell death. Cisplatin is administered through i.v. route, in a standard dosage comprised between 20 to 100 mg/m². Cisplatin can be administered in a number of ways, from daily for 5 days to once weekly every 3 to 4 weeks.

In one embodiment, the chemotherapeutic agent is gemcitabine. Gemcitabine (4-amino-1-[3,3-difluoro-4-hydroxy-5-(hydroxymethyl)tetrahydrofuran-2-yl]-1H-pyrimidin-2-one) is a nucleoside analogue. Nucleoside analogues act by being substituted to regular nucleic acids in DNA, during DNA replication, thereby leading to a dysfunctional DNA strand. This mechanism leads to arresting tumor growth, ultimately resulting in apoptosis of the targeted cells. There are different classes of nucleoside analogues, which are able to be substituted with functional nucleosides. Pyrimidine analogues such as 5-fluorouracil (5FU), floxuridine (FUDR), cytosine arabinoside (cytarabine) and gemcitabine. Gemcitabine is usually administered through i.v. route, generally in a 30 minutes infusion. Standard dosage for gemcitabine is given in an 8-week treatment cycle (one iv injection per week for 7 weeks, one week wash out), followed by one or more 4-week treatment cycle (one iv injection per week for 3 weeks, one week wash out).

The invention therefore provides a method of sensitizing a BSDL or FAPP polypeptide-expressing cell to a chemotherapeutic agent, comprising bringing the cell into contact with an antigen-binding compound of the invention. In one aspect, the invention provides a method of treating or killing a BSDL or FAPP polypeptide-expressing cell, comprising bringing the cell into contact with an antigen-binding compound of the invention and chemotherapeutic agent. In another aspect, the invention provides a method of treating a patient having a tumor, comprising conjointly administering to the patient a pharmaceutically effective amount of an antigen-binding compound of the invention and a chemotherapeutic agent.

The present invention also concerns a pharmaceutical composition comprising with an antigen-binding compound of the invention and a chemotherapeutic agent. The pharmaceutical composition can further comprise a pharmaceutically carrier.

The present invention also concerns kits comprising an antigen-binding compound of the invention and a chemotherapeutic agent. In addition, the present invention concerns a product containing an antigen-binding compound of the invention and a chemotherapeutic agent as a combined preparation for simultaneous, separate or sequential use in the treatment of a disease.

Compositions of this invention may comprise any pharmaceutically acceptable carrier or excipient, typically buffer, isotonic solutions, aqueous suspension, optionally supplemented with stabilizing agents, preservatives, etc. Typical formulations include a saline solution and, optionally, a protecting or stabilizing molecule, such as a high molecular weight protein (e.g., human serum albumin).

The invention also concerns the use of an antigen-binding compound of the invention and a chemotherapeutic agent for the preparation of a medicament for treating a disease. The present invention further concerns a method for treating a disease (e.g. pancreatic cancer) in a subject comprising administering an antigen-binding compound of the invention and a chemotherapeutic agent to the subject. The administration of the antigen-binding compound of the invention and the chemotherapeutic agent can be simultaneous, separate or sequential.

According to the methods and compositions of the present invention, compounds, preferably antigen-binding compounds of the invention and chemotherapeutic agents are administered in an “efficient” or “therapeutically effective” amount. Preferably, the therapeutically effective amount will be an amount of a therapy (e.g., a therapeutic agent) which is sufficient to ameliorate a disease or condition, or one or more symptoms thereof, or prevent the advancement of the disease or condition, or improve the therapeutic effect(s) of another therapy (e.g., a therapeutic agent or other physical treatment). Effective doses will also vary, as recognized by those skilled in the art, depending on the diseases treated, route of administration, excipient usage, and the possibility of co-usage with other therapeutic treatments such as use of other agents.

Preferably, treating an individual or subject comprises the reduction or amelioration of the progression, severity, and/or duration of a disease or condition, or one or more symptoms thereof that results from the administration of one or more therapies (e.g., one or more prophylactic and/or therapeutic agents).

Preferably, preventing a disease or condition in an individual or subject comprises the prevention of the recurrence, onset, or development of a disease or condition, or one or more symptoms. thereof in a subject, said prevention resulting from a therapy (e.g., the administration of a prophylactic or therapeutic agent), or a combination therapy (e.g., the administration of a combination of prophylactic or therapeutic agents).

In preferred embodiments, treating a cancer comprises preventing the development of a cancer, reducing the symptoms of cancer, and/or inhibiting the growth or recurrence, or reducing the size and/or inducing the destruction of an established cancer. In other aspects, a medicament is administered to a subject at risk of developing a cancer for the purpose of reducing the risk of developing a cancer.

The present invention also concerns a method of killing target cells in a subject comprising administering to the subject an antigen-binding compound of the invention and a chemotherapeutic agent. The target cells are preferably cancer cells, e.g. cells that express FAPP and/or BSDL.

The present invention also concerns a method for increasing the efficacy of a treatment with an antigen-binding compound of the invention in a subject, wherein a chemotherapeutic agent is administered to the subject prior to, simultaneously with, or following the administration of an antigen-binding compound of the invention.

In one embodiment, the chemotherapeutic agent enhances the ability of the antigen-binding compound of the invention to destroy the target cells by 10%, 20%, 30%, 40% or 0%, or more.

The present invention also comprises a method for reducing the dosage of an antigen-binding compound of the invention, by administering to an individual a chemotherapeutic agent. For example, co-administration of an antigen-binding compound of the invention and a chemotherapeutic agent allows a lower dose of the chemotherapeutic agent to be used. Such chemotherapeutic agent can be used at a 20%, 30%, 40%, 50%, 60%, 70%, 80%, 90% lower dose than the recommended dose in the absence of the compound. In another example, co-administration of an antigen-binding compound of the invention and a chemotherapeutic agent allows a lower dose of the therapeutic antibody to be used. Such therapeutic antibody can be used at a 20%, 30%, 40%, 50%, 60%, 70%, 80%, 90% lower dose than the recommended dose in the absence of the compound.

In one embodiment of the invention, the use of the antigen-binding compound of the invention allows therapeutic efficacy to be achieved with reduced doses of chemotherapeutic agent. The use (e.g., dosage, administration regimen) of chemotherapeutic agent can be limited by side effects, e.g. fever, headaches, wheezing, drop in blood pressure, and others. Accordingly, while in many patients a standard dose of the chemotherapeutic agents will be administered in combination with an antigen-binding compound of the invention, thereby enhancing the efficacy of the standard dose in patients needing ever greater therapeutic efficacy, in other patients, e.g., those severely affected by side effects, the administration of an antigen-binding compound of the invention will allow therapeutic efficacy to be achieved at a reduced dose of chemotherapeutic agents, thereby avoiding side effects. In practice, a skilled medical practitioner will be capable of determining the ideal dose and administrative regimen of the antigen-binding compound of the invention and the chemotherapeutic agent for a given patient, e.g. the therapeutic strategy that will be most appropriate in view of the particular needs and overall condition of the patient. Numerous references are available to guide in the determination of proper dosages for chemotherapeutic agents, e.g., Remington: The Science and Practice of Pharmacy, by Gennaro (2003), ISBN: 0781750253; Goodman and Gilmans The Pharmacological Basis of Therapeutics, by Hardman, Limbird & Gilman (2001), ISBN: 0071354697; Rawlins E. A., editor, “Bentley's Textbook of Pharmaceutics”, London: Bailliere, Tindall and Cox, (1977); and others.

In one embodiment, a medical practitioner can gradually lower the amount of the chemotherapeutic agent given in conjunction with the therapeutic antibody; either in terms of dosage or frequency of administration, and monitor the efficacy of the chemotherapeutic agent, monitor the presence of target cells in the patient, monitor various clinical indications, or by any other means, and, in view of the results of the monitoring, adjust the relative concentrations or modes of administration of the therapeutic antibodies and/or chemotherapeutic agent to optimize therapeutic efficacy and limitation of side effects.

Suitable doses of the chemotherapeutic agent and/or antigen-binding compound of the invention can also generally be determined in vitro or in animal models, e.g. in vitro by incubating various concentrations of antigen-binding compound of the invention in the presence of target cells, pancreatic cancer cells, and varying concentrations of chemotherapeutic agents, and assessing the extent or rate of target cell depletion under the various conditions, using standard assays (e.g. as described in the examples section). Alternatively, varying dosages of the antigen-binding compound of the invention can be given to animal models of disease (e.g. an animal model for pancreatic cancer), along with varying dosages of the chemotherapeutic agent, and the efficacy of the antibodies (e.g. as determined by any suitable clinical, cellular, or molecular assay or criterion) in treating the animals can be assessed.

The composition or product according to the present invention may be injected directly to a subject, typically by intra-venous, intra-peritoneal, intra-arterial, intra-muscular or transdermic route. Several monoclonal antibodies have been shown to be efficient in clinical situations, such as Rituxan (Rituximab) or Xolair (Omalizumab), and similar administration regimens (i.e., formulations and/or doses and/or administration protocols) may be used with the composition of this invention. The chemotherapeutic agents and antigen-binding compound of the invention can be administered by the same route or by different routes.

In another aspect, the present invention provides a method of selecting a chemotherapeutic agent for administration in combination with an antigen-binding compound of the invention, said method comprising: i) providing a candidate chemotherapeutic agent; ii) incubating the antigen-binding compound of the invention with target cells specifically recognized by the antigen-binding compound of the invention in the presence of the candidate compound; and iii) assessing the effect of the candidate compound on its to eliminate the target cells; wherein a detection that the candidate compound enhances the elimination of the target cells indicates that the candidate compound is suitable for use in the method.

Within the context of the present invention, a subject or patient includes any mammalian subject or patient, more preferably a human subject or patient.

In particular, one object of the present invention is to provide an efficient combination treatment with an antigen-binding compound of the invention according to the invention and a chemotherapeutic agent which is more effective for the elimination of pancreatic cancers cells than chemotherapy alone. In particular, another object of the present invention is to provide an efficient combination treatment with an antigen-binding compound of the invention and a chemotherapeutic agent.

In one embodiment, the therapeutic antibody and the chemotherapeutic agent are administered to the subject simultaneously. In another embodiment, the chemotherapeutic agent is administered to the subject within several week (e.g. 2, 3, 4, 5, or 6 weeks), preferably within one week of the administration of the antigen-binding compound. In one embodiment, the chemotherapeutic agent is administered to the subject before the antigen-binding compound of the invention. In a second embodiment, the antigen-binding compound of the invention is administered to the subject before chemotherapeutic agent. The chemotherapeutic agent and the antigen-binding compound of the invention are administered so that the synergic effect is obtained.

### Use of Compounds in Diagnostics or Prognostics

As demonstrated herein, the bivalent antibodies of the invention are particularly effective at detecting cells which express BDSL or FAPP polypeptides (e.g. breast cancers), because the antibodies have high affinity when in bivalent form, and without non-specific staining on tissues that do not express BDSL or FAPP polypeptides. The antibodies will therefore have advantages for use in the diagnosis, prognosis and/or prediction of pathologies involving cells which express BDSL or FAPP polypeptides, including pancreatic pathologies such as pancreatic cancer, pancreatitis and type I diabetes, and also breast cancer and cardiovascular diseases. For example, pancreatic (or breast) cancer in patients can be characterized or assessed using an antibody of the invention. This can be useful to determine whether a patient has a pathology involving cells which express BDSL or FAPP polypeptides. The method can also be useful to determine whether a patient having such a pathology can be treated with a therapy effective in cells which express BDSL or FAPP. For example the method can be used to determine if a patient will respond to an antigen binding compound that binds BDSL or FAPP (e.g. any antibody of the present invention).

The antibodies described herein can therefore be used for the detection, preferably in vitro, of a pancreatic pathology, particularly in particular pancreatic cancer. Such a method will typically involve contacting a biological sample from a patient with an antibody according to the invention and detecting the formation of immunological complexes resulting from the immunological reaction between the antibody and the biological sample. Preferably, the biological sample is a sample of pancreatic tissue as obtained by biopsy (tissue slice for a immunohistochemistry assay) or a biological fluid (e.g. serum, urine, pancreatic juices or milk). The complex can be detected directly by labelling the antibody according to the invention or indirectly by adding a molecule which reveals the presence of the antibody according to the invention (secondary antibody, streptavidin/biotin tag, etc.). For example, labelling can be accomplished by coupling the antibody with radioactive or fluorescent tags. These methods are well known to those skilled in the art. When detecting cancer, a positive determination that a FAPP or BDSL polypeptide is present in the biological sample will generally indicate that the patient is positive for the pancreatic pathology (e.g. pancreatic cancer). Accordingly, the invention also relates to the use of an antibody according to the invention for preparing a diagnostic composition that can be used for detecting a pancreatic pathology in vivo or in vitro.

The antibodies of the invention will also be useful for determining whether a subject is suitable for, or for predicting the response of a subject to, treatment with a therapeutic agent directed to a cell that expresses FAPP or BSDL polypeptide, or which is directed to a FAPP or BSDL polypeptide itself. Preferably the therapeutic agent is an antigen-binding fragment (e.g. an antibody, an antibody of the invention) that binds FAPP or BSDL polypeptide.

The antibodies of the invention will also be useful for assessing the response of a subject having cancer to a treatment with an antibody that binds FAPP or BSDL polypeptide; such a method will typically involve assessing whether the patient has cancer cells that express a FAPP or BSDL polypeptide bound by an antibody of the invention, the expression of FAPP or BSDL polypeptide being indicative of a responder subject. A positive determination that a patient has cancer cells that express FAPP or BDSL indicates that the patient will be a positive responder to treatment with an antibody that binds FAPP or BSDL polypeptide (e.g. an antibody of the invention).

Identification of responder subjects also enables methods for treating a subject having a cancer. It will be possible to assess whether the patient has cancer cells that express a FAPP or BSDL polypeptide bound by an antibody of the invention, the expression of FAPP or BSDL polypeptide bound by an antibody of the invention being indicative of a responder subject, and treating said subject whose cancer cells express a FAPP or BSDL polypeptide with an antibody that binds FAPP or BSDL polypeptide (e.g. an antibody of the invention). Assessing whether the patient has cancer cells that express a FAPP or BSDL polypeptide can be carried out for example using the diagnostic methods described herein, such as by obtaining a biological sample from a patient and contacting the sample with an antibody according to the invention and detecting the formation of immunological complexes resulting from the immunological reaction between said antibody and said biological sample. The biological sample can be a sample of pancreatic tissue (biopsy) or a biological fluid (e.g. serum, urine, pancreatic juices and milk).

Also encompassed is a diagnostic or prognostic kit for a pancreatic pathology, in particular pancreatic cancer, comprising an antibody according to the invention. Optionally the kit comprises an antibody of the invention for use as a diagnostic or progrnostic, and an antibody of the invention for use as a therapeutic. Said kit can additionally comprise means by which to detect the immunological complex resulting from the immunological reaction between the biological sample and an antibody of the invention, in particular reagents enabling the detection of said antibody.

As will be understood by those of ordinary skill in the art, the appropriate doses of chemotherapeutic agents will be generally around those already employed in clinical therapies wherein the chemotherapeutics are administered alone or in combination with other chemotherapeutics.

Further aspects and advantages of this invention are disclosed in the following experimental section, which should be regarded as illustrative and not limiting the scope of this application.

### EXAMPLES

### Materials and Methods

### Antibodies and Other Reagents

POD-labelled anti-rabbit IgG, POD-labelled anti-mouse IgG, and other antibodies to rabbit and mouse immunoglobulins were from Roche Diagnostics (Manheim, Germany), Calbiochem (San Diego, Calif.) or Cell Signaling (Beverly, Mass.). Peroxidase (POD)-conjugated goat anti-rabbit IgG and anti-mouse IgG were respectively from Cell Signaling and Calbiochem (San Diego, Calif.). Antibodies to actin and irrelevant mouse Kappa IgM, cocktail of protease inhibitors, propidium iodide and aphidicolin were from Sigma (St Louis, Mo.). Antibodies to Bcl-2 were obtained from Santa Cruz Biotechnology (Santa Cruz, Calif.). Other antibodies (cleaved caspase-3 (Asp175), caspase-3, caspase-7 (Asp175), caspase-7, cleaved caspase-9 (Asp330), caspase-9, cleaved PARP (Asp 214), PARP and Bcl-2 were from Cell Signaling (Beverly, Mass.). RPMI 1640, DMEM media, penicillin, streptomycin, trypsin-EDTA and liquid dissociation non-enzymatic were purchased from Cambrex (Cambrex Biosciences, Emerainville, France) or Lonza (Le Vallois-Perret, France). Caspase inhibitors came from Alexis (San Diego, Calif.) or Calbiochem. Antibodies directed against Bax and E-cadherin were obtained from Santa Cruz Biotechnology (Santa Cruz, Calif.). The antibody to β-catenin was obtained from Abcam (Cambridge, UK). Fluorescein isothiocyanate (FITC) conjugated goat anti-mouse IgM was from Sigma. Alexa-conjugated goat anti-rabbit IgG and anti-mouse IgG were from Molecular probes (Carlsbad, Calif.). Fumonisin B1, Fumonisin B2, L-cycloserine, Phenyl-2-decanoylamino-3-morpholino-1-propanol (PDMP), methyl-β-cyclodextrin (MβCD), filipin, Triton X-100, 3-(4,5-Dimethyl-2-thiazolyl)-2,5-diphnyl-2H-tetrazolium bromide (MTT), propidium iodide (PI) and aphidicolin were obtained from Sigma. Protease inhibitor cocktail tablets were from Roche Diagnostic (Meylan, France). DAPI (4′,6-diamidino-2-phenylindole, dihydrochloride) was from Promega (Madison, Wis.).

The monoclonal antibody mAb16D10, which recognizes peptides and may recognize the O-glycosylated C-terminal domain of the feto-acinar pancreatic protein (FAPP), an oncofetal glycolsoform of the pancreatic bile salt-dependent lipase (BSDL) and the polyclonal antibody (pAbL64), directed against human BSDL (and FAPP), were generated in our laboratory as described in WO2005/095594. All other products were of the best available grade.

### Cells and Reagents

HEK293T cells were cultured in DMEM (Gibco) supplemented with sodium pyruvate (1 mM), penicillin (100 U/ml), streptomycin (100 μg/ml) and 10% heat-inactivated FCS (PAN biotech). SOJ-6 cells were cultured in RPMI (Gibco) supplemented with sodium pyruvate (1 mM), penicillin (100 U/ml), streptomycin (100 μg/ml) and 10% heat inactivated FCS (PAN biotech). Lipofectamine 2000 reagent, Trizol, SuperScript II reverse Transcriptase and pcDNA3.1 vectors were purchased from Invitrogen.

### Cell Culture

Cell lines (SOJ-6 and Pane-1) originate from human pancreatic (adeno)carcinoma. SOJ-6 cells, which constitutively express FAPP, were grown at 37° C. in RPMI 1640 medium supplemented with 10% FCS, penicillin (100 U/ml), streptomycin (100 μg/ml), and fongizone (0.1%). PANC-1 cells that do not express FAPP were grown at 37° C. in DMEM medium supplemented with 10% FCS, glutamine (2 Mm), penicillin (100 U/ml), streptomycin (100 μg/ml), and fongizone (0.1%). The 16D10 hydridoma expressing mAb16D10 was grown at 37° C. in RPMI 1640 medium supplemented with 10% inactivated FCS, penicillin (100 U/ml), streptomycin (100 μg/rill), and fongizone (0.1%).

### Cell Death Analysis

After treatment with mAb16D10 or cisplatine in RPMI1640 with inactivated FCS, cells were harvested, washed in ice-cold PBS and resuspended in cold propidium iodide solution (0.5 mg/ml) in Isoflow buffer for 10 min at room temperature in the dark. Flow cytometry analyses were performed using Coulter FACSCalibur.

### Cell Growth and Proliferation

Cell proliferation was determined using MTT assay as previously described (Mosmann et al., 1983 J Immunol Methods, 65(1-2):55-63). Briefly, cells were seeded at subconfluence in appropriate complete media containing 10% FCS in 96-well culture plates. Media were replaced for 24 h by fresh media with 10% inactivated FCS, including increasing concentrations of antibodies directed against FAPP (pAbL64, mAbJ28 and mAb16D10). Cells were washed with PBS and incubated with 100 μl of MTT (0.5 mg/ml) in complete media for 3 h, washed with PBS and finally incubated with DMSO for 30 min at 37° C. Cell growth was determined by measuring absorbance at 550 nm using a MR 5000 microplate spectrophotometer. All independent determinations were done in triplicate and compared to control.

### Flow Cytometric Assay for CD 107 Mobilization and IFN-γ Production

Thawed purified human NK cells stimulated or not overnight with 100 UI/mL of IL-2 were mixed with SOJ-6 or B221 cell lines at an effector/target ratio equal to 1, alone or in the presence of rec16D10 (30 μg/mL) or rituxan (10 μg/mL). Cells were then incubated for 4 hours at 37° C. in the presence of FITC conjugated anti-CD107 mAbs (Becton Dickinson) and monensin (sigma). After incubation, cells were washed in PBS containing 2 mM EDTA to disrupt cell conjugates and stained for extracellular markers (PC5 conjugated anti-CD56 and PC7 conjugated anti-CD3 purchased from Beckman coulter). Cells were then fixed and permeabilized using IntraPrep reagent (Beckman Coulter). Intracellular IFN-γ was revealed using PE conjugated anti-IFN-γ purchased from Becton Dickinson. Samples were then analysed on FACScanto (Becton Dickinson).

### Flow Cytometry

Detection of antigens at the surface of SOJ-6 and PANC-1 cells was carried out by indirect fluorescence under the following conditions: cells were released from culture plates by treatment with a non enzymatic dissociation liquid (Calbiochem) for 15 minutes at 37° C. All subsequent steps were carried out at 4° C. The cells were washed with phosphate buffered saline (PBS), fixed with 2% paraformaldehyde in PBS for 15 minutes, and washed with 1% BSA in PBS for 15 minutes. Antigens were exposed for 2 hours to specific antibodies, washed with PBS, and finally incubated for 45 minutes with appropriate FITC-labeled secondary antibodies. Cells were then washed, resuspended in isoflow buffer, and analyzed on Coulter FACSCalibur device.

Cells were washed twice using cold PBS 1×/BSA 0.2%/Sodium Azide 0.05% buffer. Staining was performed using the different antibodies during 1H at 4° C. into round 96-well plates using 5×10⁴ cells per well. Cells were then washed twice before being incubated with secondary reagents. After two washes, cells were re-suspended before acquisition into PBS1X/Formaldehyde 1%. Stainings were acquired on a FACScan (Becton Dickinson, San Jose, Calif.) and results were analysed using FlowJo Software. SOJ-6 cells pre-treated or not with 5 μg/ml aphidicolin (a reversible inhibitor of eukaryotic nuclear DNA replication, which blocks the cell cycle at early S-phase) for 6 h, were then incubated with mAb16D10 or irrelevant IgM used as a negative control. Cells were released from culture plates with a non-enzymatic cell dissociation solution, washed with PBS^{+/+}, fixed with 70% ethanol at −20° C. and washed with PBS^{+/+}. The cells were resuspended in a solution of 400 μg/ml propidium iodide in isoflow buffer and were incubated for 30 min at room temperature as already described [Mi-Lian et al., 2004]. Cell-cycle distribution was detected by flow cytometry and analyzed by Mod Fit software (Verity Software House, Inc., Topsham, Me.). The red fluorescence of single events was recorded using excitation and emission at 488 nm and at 610 nm respectively, to measure the DNA index.

RNA Extraction and cDNA Preparation from the 16D10 Hybridoma

16D10 hybridoma cells (5×10⁶ cells) were re-suspended in 1 ml of Trizol reagent. RNA extraction was performed by adding 200 μl chloroform. After centrifugation (15 min, 13,000 rpm), RNA was precipitated from the aqueous phase with 500 μl isopropanol. After incubation (10 min, RT) and centrifugation (10 min, 13,000), RNA was washed with 70% ethanol and re-centrifugated (5 min, 13,000 rpm). RNA was re-suspended in H₂O (Rnase-free water). cDNA was obtained using SuperScript II reverse Transcriptase using 2 μg of specific RNA and following manufacturer's instructions. cDNA quality was checked by PCR reaction using 5′ TG AAG GTC GGT GTG AAC GGA TT (SEQ ID NO: 17) and 3′ CTA AGC AGT TGG TGG TGC AGG AT (SEQ ID NO: 18) oligonucleotides to amplify GAPDH.

### Cloning of the VH and VL Domain of the 16D10 Antibody

VL-Ck domains of the 16D10 antibody were amplified by PCR from cDNA using 5′ AAGCTAGCATGGAATCACAGACTCAGGCT (SEQ ID NO: 19) and 3′AAGCGGCCGCCTAACACTCATTTCTGTTGAAG (SEQ ID NO: 20) oligonucleotides. After TA-cloning and sequencing, the sequence was cloned into pcDNA3.1 vector between NheI and NotI restriction sites. VH-CH1 domains of the 16D10 antibody were amplified by PCR from cDNA using 5′ AAGAATTCATGGAATGGAGCTGGGTCTTTC (SEQ ID NO: 21) and 3′ AAGGTACCTGGAATGGGCACATGCAGATC (SEQ ID NO: 22) oligonucleotides. After TA-cloning and sequencing, the sequence was cloned into the 958 cosFClink vector between the EcoRI and KpnI restriction sites.

### Transfection

HEK-293T cells were seeded 24 hours prior to transfection into 75 cm² flasks (5×10⁶ cells/flask) in DMEM without antibiotics. Transfections were performed using 15 μg of the pcDNA3.1/VL-Ck constructs and 15 μg of the 958 cosFClink/VH-CH1 constructs using Lipofectamine 2000 according to manufacturer's instructions. To ensure DNA purity for transfection, the Maxi-prep endotoxin-free kit from Qiagen was used. The Lipofectamine:DNA ratio used was fixed at 2:1. Culture supernatant were harvested after 4, 8, and 12 days of transfection.

### Purification of the Antibody

The 16D10 was purified from the supernatant using protein-A sepharose CL-4B beads (GE Healthcare). Batch purification was performed under rotation at 4° C. The beads were then centrifuged 5 min at 4° C. at 1500 rpm before being loaded onto a column. After extensive washes in 1×PBS, the antibodies were eluted using glycine 0.1 M pH 3 buffer before being dialyzed overnight at 4° C. against 1×PBS buffer.

### SDS-PAGE and Western Blots

SOJ-6 cells were grown in 6-well culture plates in RPMI 1640 medium with 10% FCS. At subconfluency, the medium was removed and replaced for 24 hours by fresh RPMI medium with 10% inactivated FCS. SOJ-6 cells were then incubated with mAb16D10 or Cisplatin for 24 hours. At the end of incubation, cells were washed three times with ice-cold PBS (without Na⁺ and Mg⁺⁺) harvested and pelleted by centrifugation. Pellets were washed twice and lysed at 4° C. in 0.5 ml of lysis buffer (10 mM Tris-HCl pH 7.4, 150 mM NaCl, 1% Triton X-100, 1 mM benzamidine and phosphatase inhibitors). After lysis, homogenates were clarified by centrifugation at 10,000 g for 10 min at 4° C. An aliquot was saved for protein determination using the bicinchoninic acid assay (Pierce, Rockford, Ill.). Proteins (50 μg/lane) in reducing SDS buffer were separated onto 10, 12, or 15% polyacrylamide with 0.1% SDS (according to the molecular weight range of proteins to be separated). After electrophoretic migration, proteins were silver stained. Alternatively, proteins were transferred onto nitrocellulose membranes using a Mini Transblot electrophoretic cell (BioRad, Hercule, Oreg.), and transferred proteins were immunodetected by using appropriate primary and secondary antibodies. After washes, membranes were developed with a chemoluminescent substrate according to the manufacturer's instructions (Roche Diagnostics, Switzerland). In each experiment, a control was included by omitting primary antibodies or by using a non-immune serum.

### Apoptosis and Caspase Activities

Cells grown in 8-well plates (Polystyrene vessel, BD Falcon) were treated with mAb16D10 or mouse IgM in RPMI with inactivated FCS for 24 hours prior to the addition of CaspACE FITC-VAD-fmk in situ marker (Promega) at a final concentration of 10 μM in the culture medium according to manufacturer's instructions. Then cells were washed in PBS, fixed for 15 minutes in 2% paraformaldehyde, and washed once again. Upon caspase action on FITC-VAD-fmk, apoptotic cells become fluorescent and the number of fluorescent cells was determined in triplicate on collections of 10 fields randomly examined under the fluorescent microscope.

Apoptosis assay for Example 14 (Apoptosis by Chimeric Recombinant 16D10)

SOJ-6 cells (20.10⁴ cells per well) were seeded onto 24 wells plates 72H before to start the experiment. Cells were incubated either with 20 μg/ml 16D10 IgM, or 20 μg/ml of a further recombinant 16D10 IgG1 antibody which contained the 16D10 variable regions linked fused to a human IgG1 constant region and human kappa light region for the heavy and light chains respectively, 25 μg/ml Tunicamycin or 50 μg/ml Tunicamycin. The AnnexinV/PI stainings were performed after 24H of culture using the AnnexinV-FITC Apoptosis Detection Kit I (BD Pharmingen) according to the manufacturer instructions. Stainings were acquired on a FACScan (Becton Dickinson, San Jose, Calif.) and results were analysed using FlowJo Software.

### Nuclear Staining

After treatment with mAb16D10 or cisplatin, cells were washed in ice-cold PBS, fixed and permeablized with 70% ethanol for 5 min at ±20° C. and staining with diluted 1/1000 DAPI solution in PBS for 1 min at room temperature. The cells were then washed with PBS. The nuclear morphology of cells was examined by fluorescence microscopy.

### Statistical Analysis

All data are presented as mean±SD. Significant differences among the groups were determined using the unpaired Student's t-test. Values of *P<0.01 were accepted as statistically significant.

### Example 1

### Pancreatic SOJ-6 Cells Treated with mAb 16D10 Undergo Cellular Death by Apoptosis Over 24H

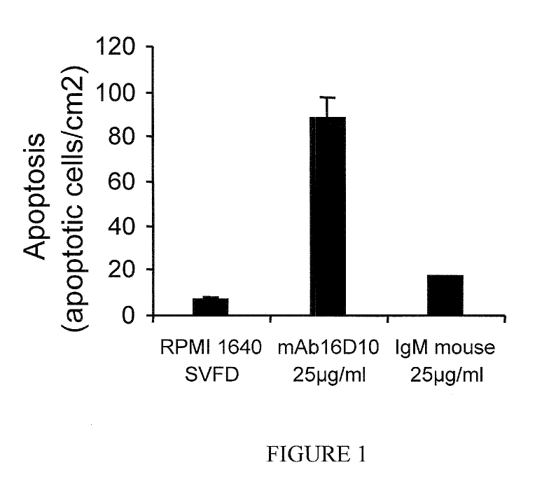

The ability of mAb16D10 to stimulate apoptotic cellular death of SOJ-6 cells was investigated as described herein. It was observed that antibody 16D10 leads to the apoptosis of SOJ-6 cells (compared to RPMI and mouse IgM, as shown in FIG. 1, the y-axis representing the number of apoptotic cells/cm2).

### Example 2

### 16D10 Induced Apoptosis is Mediated by Caspase-3, Caspase-8, and Caspase-9 Activation

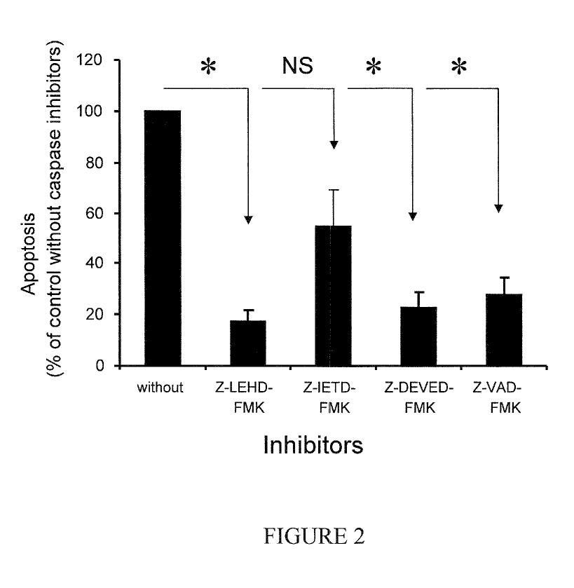

In this experiment, apoptosis induced by 16D10 was measured with CaspAce FITC-VAD-fmk on Pancreatic SOJ-6 cells pre-treated with or without caspase inhibitors, (caspase 9: Z-LEHD-fmk, caspase8: Z-IEDT-fmk, caspase3: Z-DEVED-fmk, and caspase mix: Z-VAD-fink), and then treated with mAb16D10. FIG. 2 shows that mAb 16D10 stimulates apoptosis through the caspase-3, caspase-8, and caspase-9.

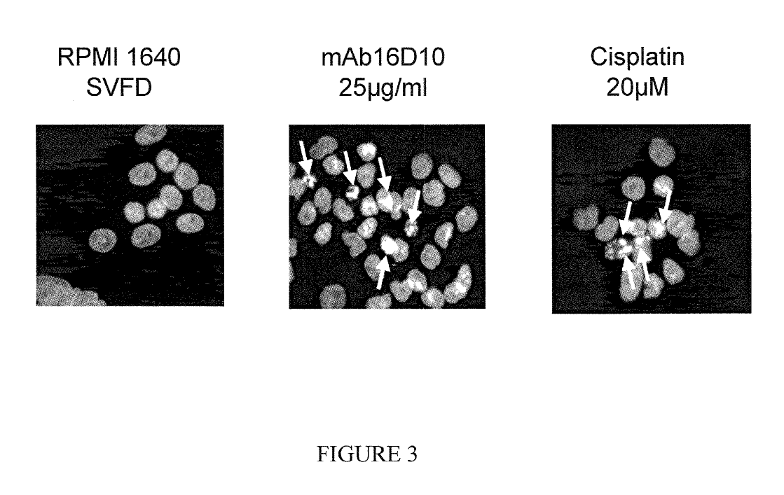

Apoptosis of SOJ-6 cells induced by mAb16D10 was also observed by DAPI staining. Results are shown in FIG. 3, where RPMI induced no apoptosis on cells, Cisplatin induced a low level of apoptosis, and antibody 16D10 induced significant levels of apoptosis, as observed by light coloration on cells in FIG. 3 corresponding to nuclear fragmentation.

### Example 3

### 16D10 is Controlled by the Bcl-2 Family of Proteins

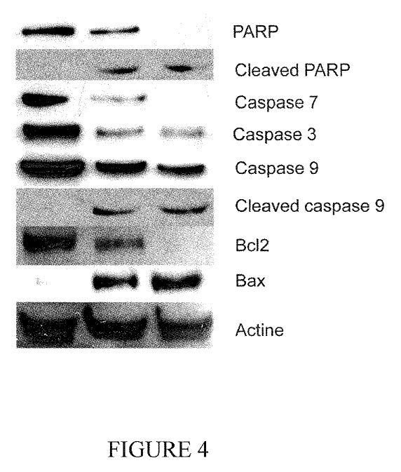

Using SDS-PAGE and western blotting as described herein it was observed that treatment of cells with 16D10 induces a decrease of the anti-apoptotic protein Bcl-2 associated with an increase of Bax protein, indicating that the caspase activation is controlled by the Bcl-2 family of proteins. The experiment also demonstrated that 16D10-induced apoptosis is mediated via caspases 8 and 9, and poly-ADP ribose polymerase (PARP) cleavage. FIG. 4 shows the results on a gel, where in the leftmost lane represents SOJ-6 cells in RPMI, the middle lane represents SOJ-6 cells incubated with antibody 16D10, and the rightmost lane represents SOJ-6 cells incubated with cisplatin.

### Example 4

### mAb 16D10, but not Antibodies pAbL64 and mAb J28, all of Which are Directed Against BDSL and/or FAPP, can Inhibit Pancreatic Tumor Cell Growth

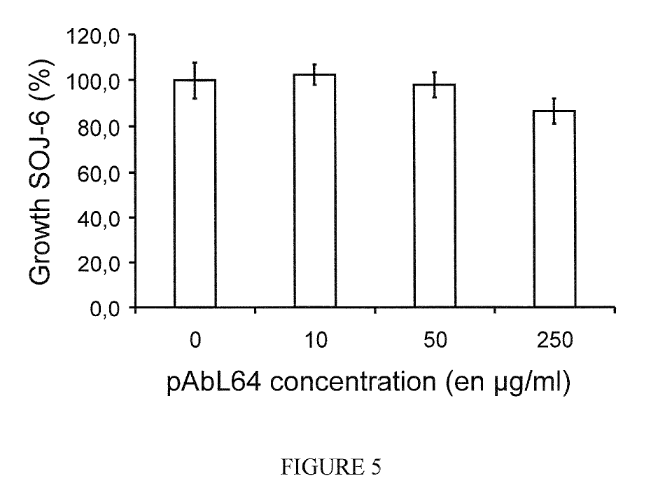

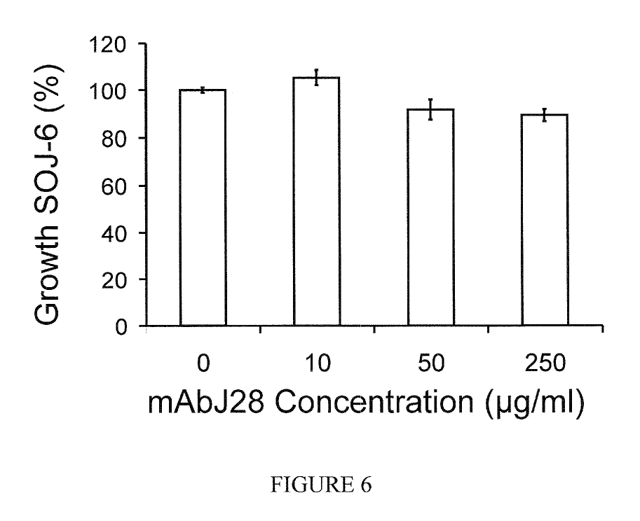

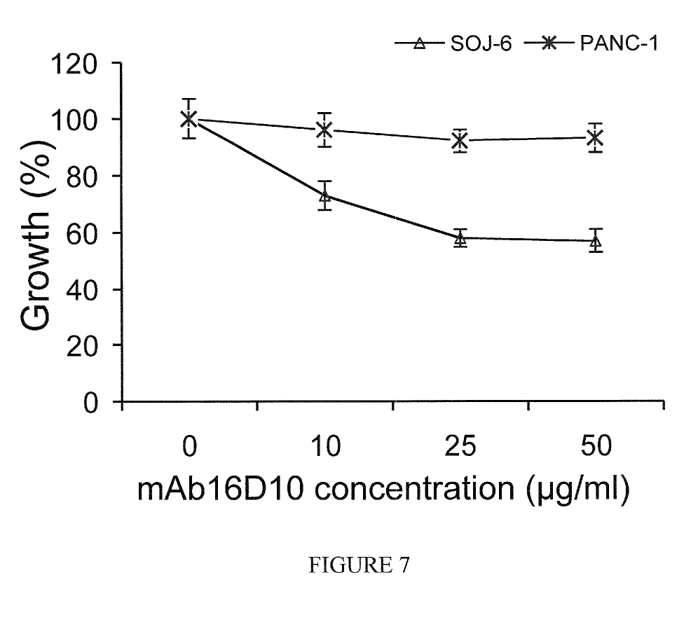

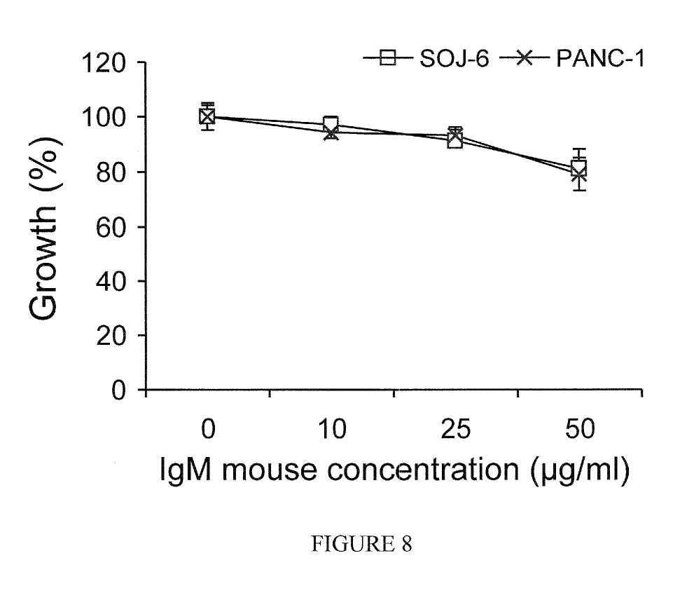

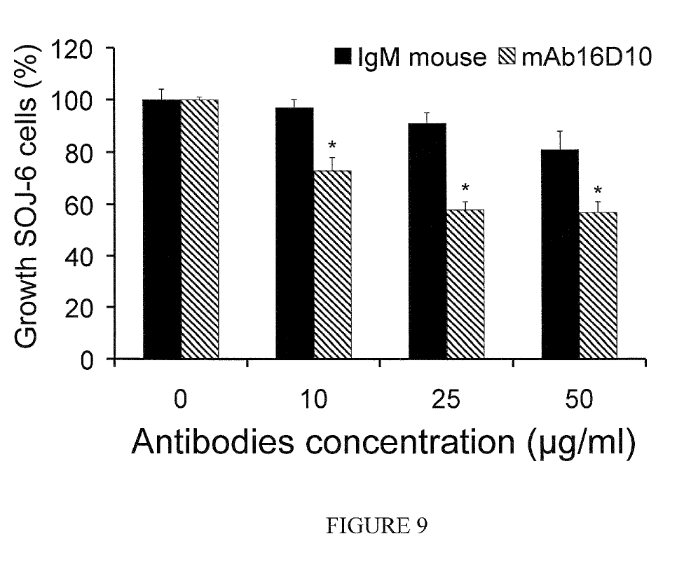

Cell growth was assessed using the MTT assay described herein. FIG. 5 shows treatment of SOJ-6 pancreatic tumor cells with increasing concentrations of polyclonal antibody pAbL64 which recognizes human BDSL and/or FAPP. FIG. 5 shows that pAbL64 is unable to cause a decrease in growth or number of cells (x-axis is mAb concentration and y-axis is % growth of cells). FIG. 6 shows treatment of SOJ-6 pancreatic tumor cells with increasing concentrations of polyclonal antibody J28 which recognizes human BDSL and/or FAPP, but which has been demonstrated previously by the inventors to bind a different epitope on BDSL and/or FAPP from antibody 16D10. FIG. 6 shows that J28 is unable to cause a decrease in the growth or number of cells (x-axis is mAb concentration and y-axis is % growth of cells). FIG. 7 shows treatment of SOJ-6 or PANC-1 pancreatic tumor cells with increasing concentrations of polyclonal antibody 16D10 (IgM) which recognizes human BDSL and/or FAPP. FIG. 7 shows that 16D10 is unable to cause a decrease in growth or number of PANC-1 cells which do not express 16D10 antigen but does cause a decrease in SOJ-6 cells which do express FAPP (x-axis is mAb concentration and y-axis is % growth of cells). FIG. 8 shows treatment of SOJ-6 or PANC-1 pancreatic tumor cells with increasing concentrations of a control IgM antibody showing that control IgM antibody is unable to cause a decrease in growth or number of neither PANC-1 nor SOJ-6 cells (x-axis is mAb concentration and y-axis is % growth of cells). FIG. 9 shows treatment of SOJ-6 pancreatic tumor cells with increasing concentrations of either antibody 16D10 or control IgM antibody, demonstrating that 16D10 causes decrease in cells while control IgM antibody does not (x-axis is mAb concentration and y-axis is % growth of cells).

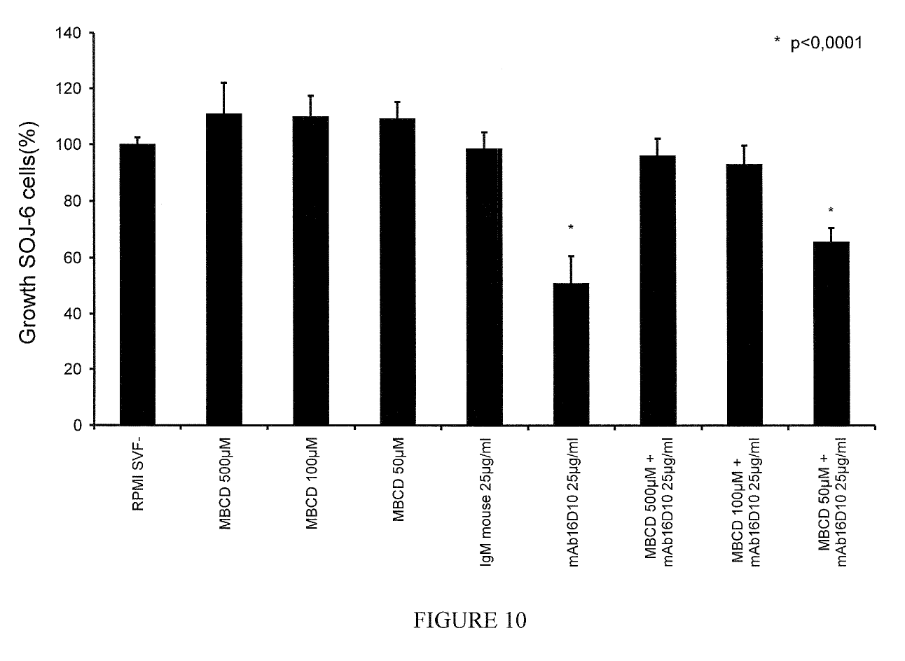

FIG. 10 shows treatment of SOJ-6 pancreatic tumor cells with increasing concentrations of either antibody 16D10 or control IgM antibody, and methyl-b-cyclodextrin (MBCD) at various concentrations with or without antibody 16D10, demonstrating that 16D10 causes decrease in cells while control IgM antibody does not (x-axis is mAb concentration and y-axis is % growth of cells). MBCD when used in combination with 16D10 decreases or abolishes the cell growth inhibiting activity of antibody 16D10. These data indicate that the ability of mAb to stimulate apoptotic cellular death is dependent on the localization of the 16D10 antigen in membrane lipid raft microdomains.

### Example 5

### mAb16D10 Regulates the Cell Cycle of SOJ-6 Cells and the Expression of Cell Cycle Regulatory Proteins

We next wished to know whether mAb16D10-induced apoptosis was due, in part, to the arrest of the cell cycle. Cell cycle distribution of the SOJ-6 cells after treatment was assessed by observing DNA profiles following SOJ-6 cells pre-treated not or with aphidicolin, treated not or with mAb16D10. Each experiment was carried out in triplate. We used a specific DNA marker, Propidium Iodide, to determine the different phases of the cell cycle by flow cytometry. Treatment of SOJ-6 cells with mAb16D10 resulted in both a G1/S arrest (G1/S: 96%) and an increase in apoptotic cells (6%), whereas the percentage of cells in G2/M phase decreased (4%). These results were confirmed when cells were synchronized in G1/S phase by aphidicolin. Indeed, the shift of cells from the G2/M to the G1/S phase and from the G1/S to the apoptosis was also observed following aphidicolin treatment. In this latter case, since cells were blocked in G1/S phase, the shift occurred from the S phase to the G0/G1 phase and from the G0/G1 phase to the apoptosis.

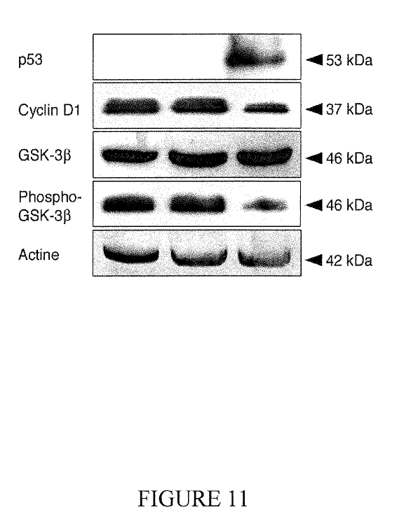

We performed the same experiment on PANC-1 cells, and no effect on the cell cycle was observed upon mAb treatment. The expression of different cell cycle regulatory proteins, specifically p53 and cyclin D1, was next analyzed. As expected, treatment of cells with mAb16D10 increased the expression of p53 and decreased that of cyclin D1 (FIG. 11). Since cyclin D1 expression may be directly regulated by GSK-3β (Diehl et al., 1998 Genes Dev. 12(22):3499-511), we next focused on the expression level of this kinase in SOJ-6 cells once challenged with mAb16D10. Although the expression of total GSK-3β was constant, a decrease in the phospho-GSK-3β (inactive form) was observed upon incubation of cells with mAb16D10 (FIG. 11). Together, these results suggest that treatment of cells with mAb16D10 induces an activation of GSK-3β leading to the degradation of cyclin D1 and resulting in arrest of cells in G1/S phase.

### Example 6

### Disorganization of Membrane Raft Structure Decreases The Antiproliferative Effect of mAb16D10

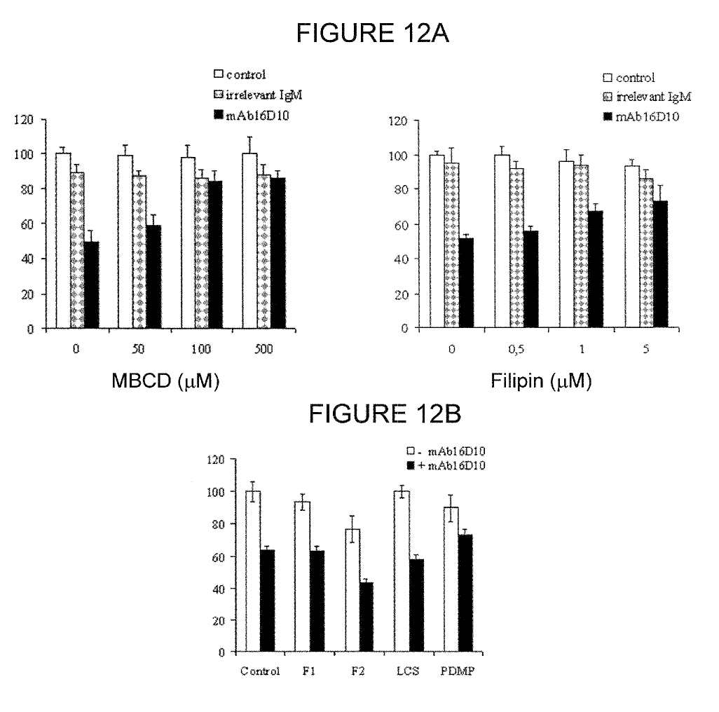

Several studies have shown that BSDL is associated with raft lipid domains on human pancreatic SOJ-6 tumoral cell surface (Aubert-Jousset et al., 2004 Structure, 12(8):1437-47). Pharmacological manipulation of membrane lipid domains with well-documented drugs has been used to address the role of lipid rafts in many systems. For this purpose we used methyl-β-cyclodextrin (MβCD) and Filipin, drugs described to deplete cholesterol in membrane rafts or to sequester cholesterol, respectively (Chen et al., 2002 J Biol. Chem.; 277(51):49631-7). As illustrated in FIG. 12A, the antiproliferative effect of mAb16D10 decreased in presence of methyl-β-cyclodextrin and Filipin.

Sphingolipids also participate in raft structures; therefore, we used metabolic inhibitors of (glyco)sphingolipid biosynthesis (Aubert-Jousset et al., 2004). Although tested at an efficient concentration (10 μM), neither L-cyclo-serine (LCS) (an inhibitor of serine palmitoyltransferase) nor Fumonisin 1 or 2 (both inhibitors of dihydroceramide synthetase) interfered with the antiproliferative effect of mAb16D10 (FIG. 12B). We next tested Phenyl-Decanoyalimino-Morpholino-Propanol (PDMP), an inhibitor of glycosphingolipid synthesis, acting on the last step of sphingolipid synthesis (Lefrancois et al., 2002, J Biol. Chem. 277(19):17188-99). As illustrated in FIG. 12B, PDMP impaired the effect of mAb16D10 on SOJ-6 cell proliferation. These results indicate that the 16D10 antigen is likely located in cholesterol-rich microdomains and that this association of 16D10 antigen with these raft microdomains could be necessary to induce apoptosis. However, the neo-synthesis of these microdomains did not appear to be involved in this pathway. Consequently, the integrity of cholesterol-rich microdomains is a prerequisite to the presence of 16D10 antigen at the surface of pancreatic tumoral cells.

### Example 7

### mAb16D10 Regulates E-Cadherin Expression and B-Catenin Localization in SOJ-6 Cells

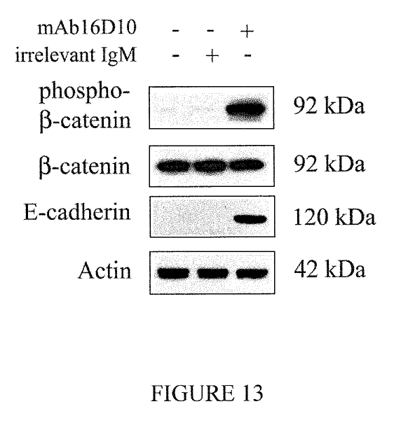

Roitbak et al. (2005) showed that β-catenin and E-cadherin complexes are associated with the lipid raft marker Caveolin-1 in human kidney epithelial cells. These molecules might confer to these lipid raft domains the role of signalling. Immunoblottings were performed to examine the expression of E-cadherin and of β-catenin by pancreatic tumoral cells. As illustrated in FIG. 13, lysate from SOJ-6 cells treated with mAb16D10 exhibited high E-cadherin protein expression in contrast to cells treated with irrelevant IgM. The overexpression of E-cadherin at the plasma membrane of the SOJ-6 cells in response to mAb16D10 treatment was demonstrated by immunofluorescence microscopy, where SOJ-6 cells were treated with or without mAb16D10 for 24 h, washed, fixed with paraformaldehyde and saturated with 1% BSA and 0.05% saponin in PBS, further incubated with primary antibodies (anti-E-cadherin, anti-β-catenin, anti-phospho-(β-catenin), following secondary antibodies FITC 488 nm or Alexa 594 nm. PANC-1 cells treated or not with mAb16D10 did not express E-cadherin at their plasma membrane (data not shown). This result suggests that overexpression of E-cadherin is dependent on the presence of 16D10 antigen at the cell surface and the treatment with mAb16D10. However, mAb16D10 did not induce a significant change in the expression of β-catenin (FIG. 13).

To determine whether treatment with mAb16D10 could affect the localization of J3-catenin, fluorescence microscopy analysis was performed next. β-catenin was found in the cytosolic compartment after SOJ-6 cell treatment with mAb16D10 whereas it was localised at the plasma membrane in untreated cells. Several studies have shown that, in the absence of Wnt signalling, the phosphorylation of residues of β-catenin addressed this protein to degradation by the ubiquitin-dependent proteasome pathway (Aberle et al., 1997 EMBO J. 16(13): 3797-804 and Orford et al., 1997 Biol. Chem. 272(40): 24735-8). Indeed, β-catenin was phosphorylated in cells treated with mAb16D10 (FIG. 13), suggesting that β-catenin cannot translocate to the nucleus to activate target genes such as cyclin D1 and instead should be degraded. Furthermore, β-catenin may be regulated by GSK-3β, which itself is activated in cells once treated with mAb16D10 (FIG. 11). These results confirm our previous experiments showing that mAb16D10 evokes a cell cycle arrest in phase G1/S. These results show that mAb16D10 inactivates β-catenin and restores directly or indirectly expression of E-cadherin in human pancreatic tumoral cells. Lastly, we wanted to determine whether the association of E-cadherin, β-catenin, and 16D10 antigen in the rafts microdomains is required for mAb16D10 to induce apoptosis (FIG. 13).

### Example 8

### SOJ-6 but not PANC-1 Cells Express the Antigen Recognized by 16D10

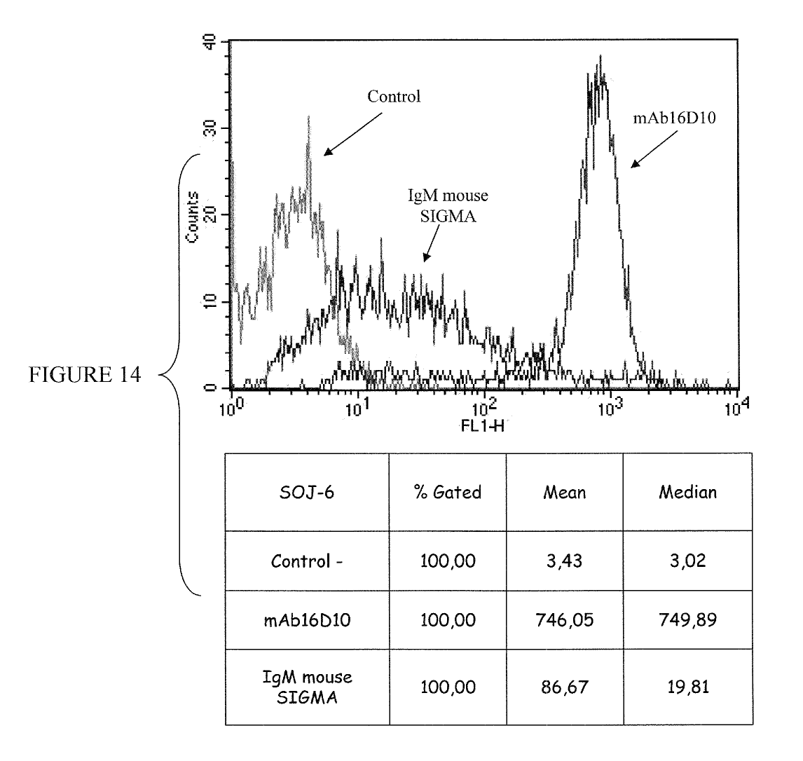

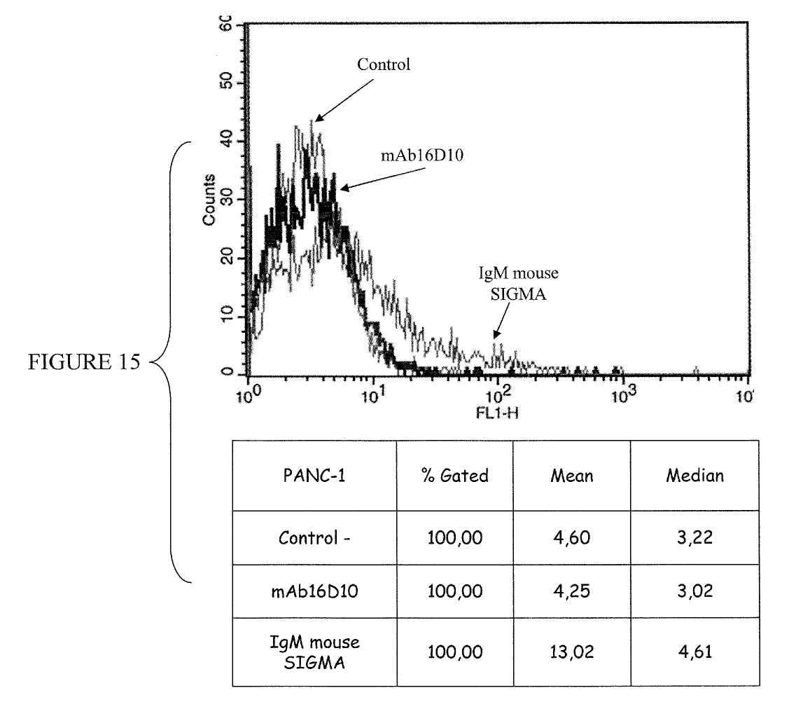

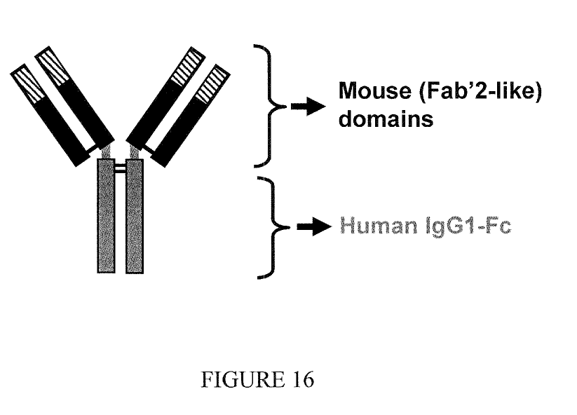

As demonstrated herein, antibody 16D10 is able to inhibit cell growth in SOJ-6 cells but not in PANC-1 cells. Flow cytometry experiments were carried out to investigate whether the antigen recognized by 16D10 was found on the surface of cells. Results are shown in FIGS. 14 and 15, representing SOJ-6 and PANC-1 cells, respectively. In FIG. 14, antibody 16D10 was found to bind antigen present on SOJ-6 cells, and in FIG. 15§, antibody 16D10 did not bind antigen present on PANC-1 cells. In each case, 16D10 binding was compared to a negative control and a control mouse IgM (Sigma). The x-axis shows fluorescent intensity and the y-axis shows counts.

### Example 9

### Production of a Bivalent 16D10 Chimeric Antibody in HEK293T Cells

cDNAs corresponding to the VH and VL chains of the mouse 16D10 antibody were obtained by RT-PCR amplification of hybridoma DNA. H: VH and CH1 domains were amplified, cloned, sequenced and subcloned into the COS-fc-link vector in frame with human IgG1-Fc. L: VL and Ck domains were amplified, cloned, sequenced and sub-cloned into the pcDNA3 expression vector.

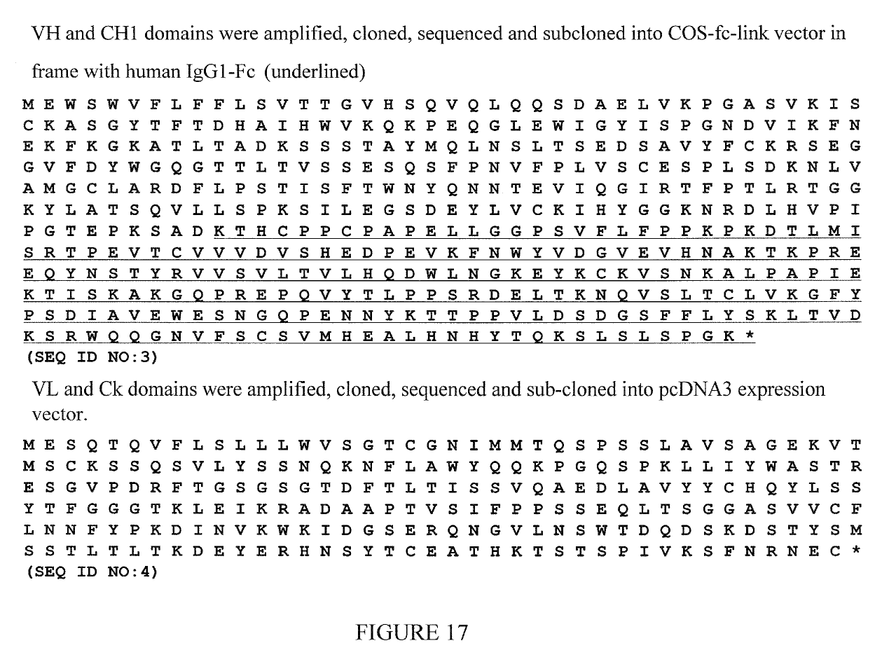

A chimeric antibody was produced comprising the variable (Fab2′-like) domains of the mouse 16D10 antibody and the constant (Fc) domains of a human IgG1 antibody. This antibody is referred to as rec16D10. Antibodies were produced in HEK293T cells, either transiently (by co-transfection of 958COS-Fc-link-VH-16D10 and pcDNA3-VL-16D10 vectors) or stably (by co-transfection using pcDNA6-Fc-VH-16D10 and pcDNA3-VL-16D10 vectors). The purity and yield of the produced antibodies were confirmed by SDS-PAGE analysis after Prot-A purification, and the activity was confirmed by FACS on SOJ-6 cells. See FIGS. 16, 17.

The IgM 16D10 antibody and the chimeric rec16D10 were each incubated with trypsin to investigate its effect on binding to SOJ-6 cells. Trypsin was found to substantially decrease the binding of both antibodies to the cells.

### Example 10

### Internalization of IgM 16D10

A pulse-chase experiment using confocal microscopy was used to assess the interaction of the IgM 16D10 antibody with living cells in culture. (Pulse: 30 min at 4° C.; Chase: 0 or 2 h at 37° C. in culture medium.) It was observed that virtually all of the mAb was internalized within 2 h. A fraction of the mAbs co-localized with LAMP1.

### Example 11

### Effect of Antibodies on Cell Proliferation

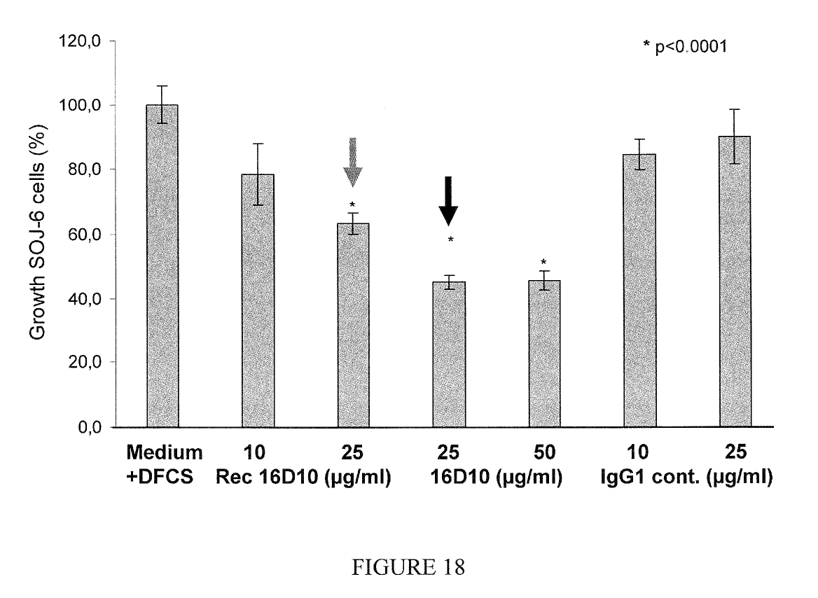

The effects of the IgM antibody 16D10 and the chimeric antibody rec16D10 on the proliferation of SOJ-6 cells were examined. Cells were incubated in culture with no antibodies, with various amounts of 16D10 or rec16D10 antibodies, or with an irrelevant IgG antibody. Antibody 16D10 reduced cell proliferation by approximately 50% at either 25 or 50 μg/ml, while rec16D10 reduced proliferation by more than 20% and more than 35% at 25 and 50 μg/ml, respectively (FIG. 18). Accordingly, both Rec 16D10 and 16D10 IgM had a direct negative effect on SOJ-6 proliferation.

### Example 12

### Examining the Ability of Rec16D10 to Activate NK Cells (ADCC)

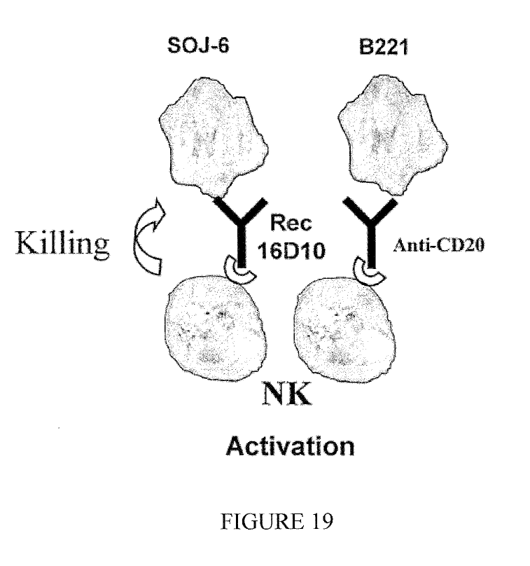

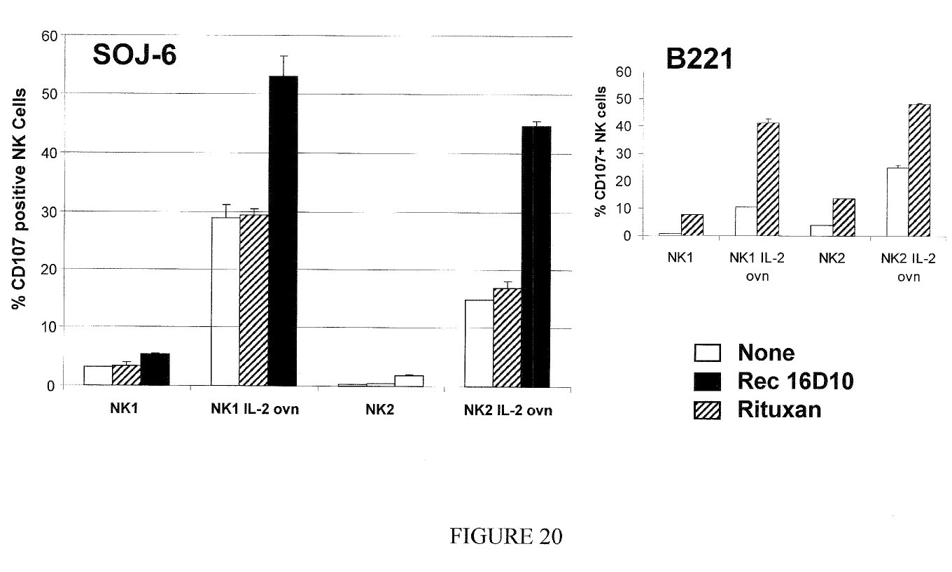

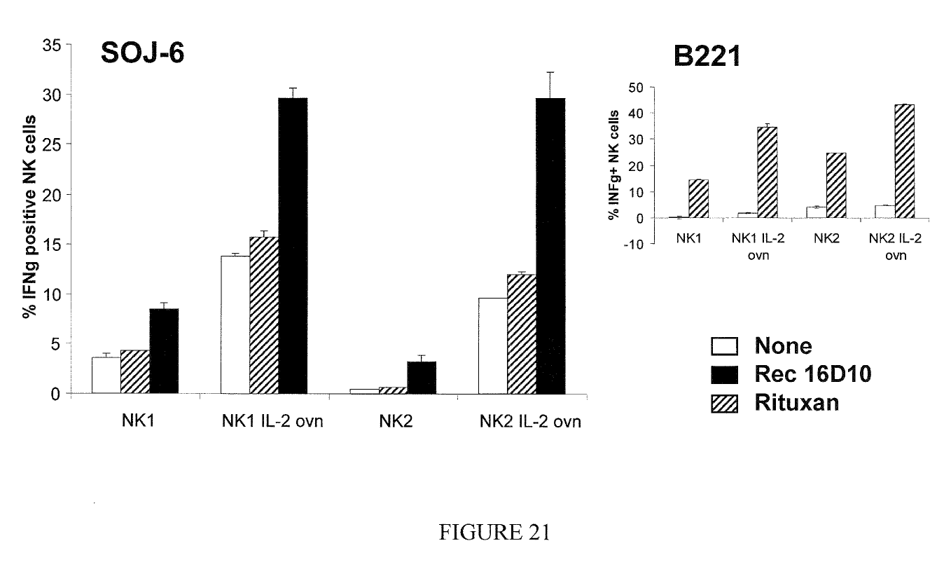

The chimeric antibody rec16D10 (30 μg/ml) was incubated with target SOJ-6 cells together with NK cells, with or without overnight treatment with IL-2 (100 U/ml) (FIG. 19). As a control, the Rituxan antibody (10 μg/ml) was used with target B221 cells. Effector cells and target cells were used at a ratio of: 1/1 (100000 NK/well). Thawed purified NK cells from two donors (NK1 and NK2) were incubated with the antibodies and target cells. Activation of NK cells was examined by virtue of CD 107 staining and IFN-γ secretion. Following IL-2 treatment and in the presence of SOJ-6 cells, rec16D10 induced CD 107-positive staining in approximately 53% and 45% of NK1 and NK2 cells, respectively (vs. <30% and <20% in controls with no antibody or with Rituxan) (FIG. 20). Under similar conditions, approximately 30% of NK1 and NK2 cells secreted IFN-γ (vs. less than 16% and 13% in the absence of antibody or with Rituxan in NK1 and NK2 cells, respectively) (FIG. 21). These results demonstrate that the bivalent antibody (with human IgG Fc portion) rec16D10 can effectively activate NK cells in the presence of target cells and can thus induce cell mediated killing of target cells (ADCC).

### Example 13

### Tissue Specificity of IgM 16D10 and IgG Rec16D10 Antibodies

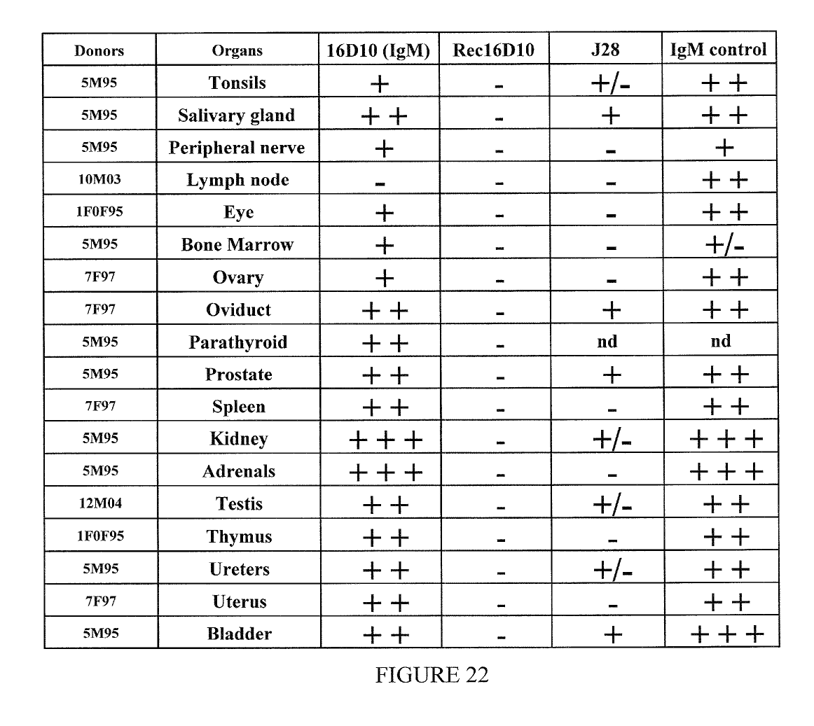

The staining specificity of the IgM 16D10 and chimeric IgG rec16D10 antibodies was assessed by examining their respective staining of various healthy tissues. The IgM antibody 16D10 exhibited positive staining on a number of tissues, including tonsils, salivary gland, peripheral nerve, eyes, bone marrow, ovary, oviduct, parathyroid, prostate, spleen, kidney, adrenals, testes, thymus, ureters, uterus, and bladder. Staining with the chimeric antibody reel 6D10, in contrast, was negative on each of these healthy tissues. Therefore, the chimeric IgG antibody rec16D10 is superior to the IgM antibody 16D10 with respect to the lack of non-specific crossreactivity (FIG. 22).

### Example 14

### Binding of 16D10 and Rec16D10 to Fixed SOJ-6 Cells

In preliminary FACS experiments, it was observed that the regular 16D10 mAb staining seemed stable on fixed SOJ-6 cells, indicating that the IgM form binds to cell surface antigens with good avidity. For the bivalent 16D10 form, cell surface binding was less stable, with an average half-life of about 80 minutes. Taking in account that most of the antibodies that have been studied so far at Innate Pharma, have kₒₙ rate association constants ranging from 5×10⁵ to 5×10⁶ M⁻¹s⁻¹, one can estimate that the recombinant 16D10 antibody bivalent affinity is in the nanomolar order (e.g., 10 to 1 nanoM) which is compatible with the industrial development of a therapeutic antibody.

### Example 15

### Induction of Apoptotis of SOJ-6 Cells Using a Recombinant 16D10 IGG1

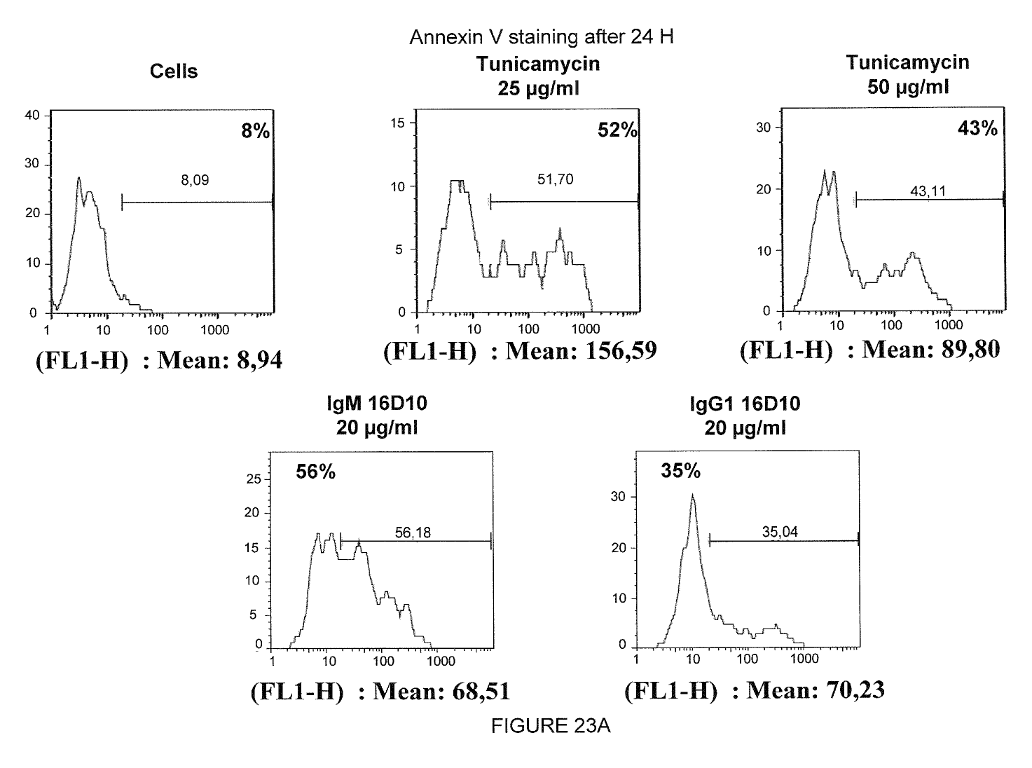

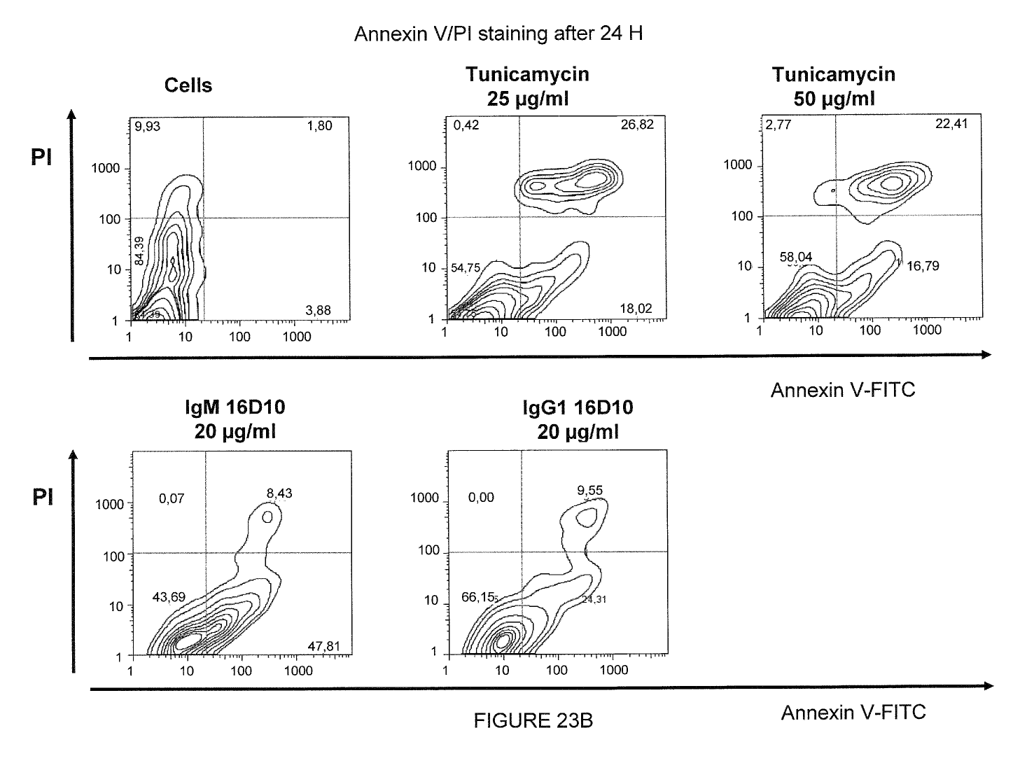

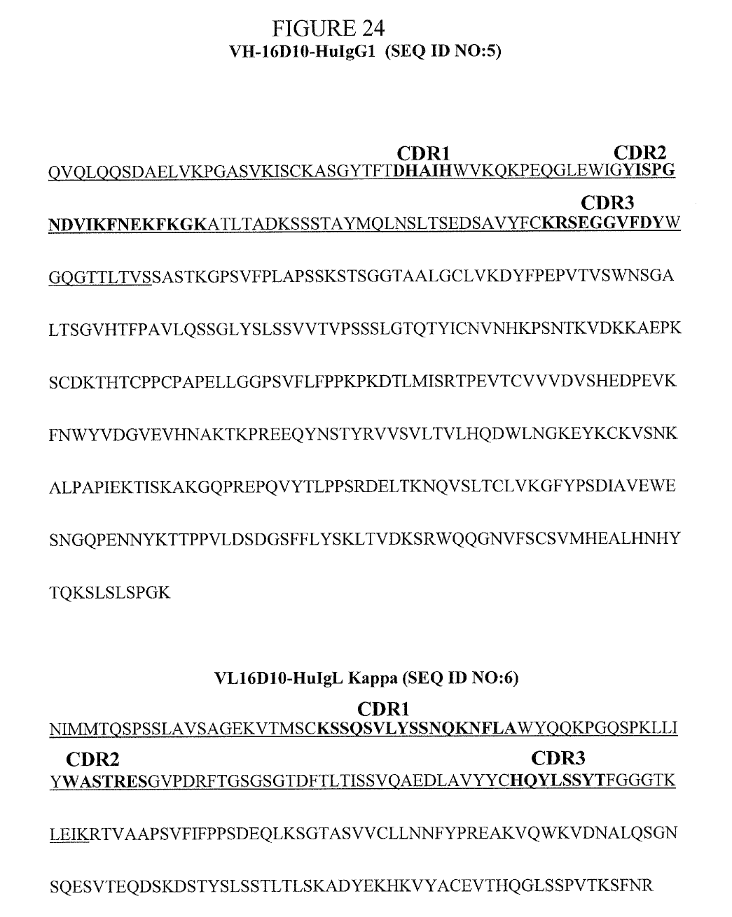

In preliminary experiments, it was observed that a bivalent, chimeric recombinant 16D10 antibody is capable of inducing apoptosis of SOJ-6 cells, as assessed by Annexin V and Annexin V/PI staining following culture for 2 hours. Both the IgG1 and IgM forms of 16D10 induced apoptosis of SOJ-6 cells. Apoptotic activity of the two antibodies was compared with tunicamycin and without treatment as a control. Results are shown in FIG. 23.

### Example 16

### Effect of Gemcitabine and Cisplatin on Proliferation of Human Pancreatic Tumor Cells

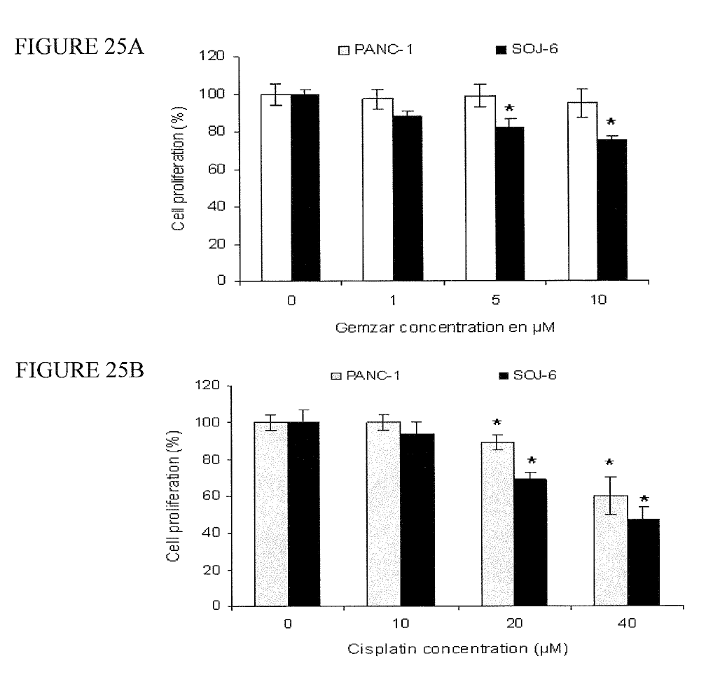

SOJ-6 and PANC-1 cells were treated with gemcitabine (Gemzar™) or cisplatin and proliferation was assessed by MTT assay after 24 hours, using increasing concentrations of gemcitabine (0 to 10 μM) or cisplatin (0 to 40 μM). Results for gemcitabine and cisplatin are shown in FIGS. 25A and 25B, respectively. The results show that a dose dependent decrease in proliferation is seen for both cell lines with cisplatin. For gemcitabine, however, a decrease in proliferation is seen only in SOJ-6 cells, as PANC-1 cells are relatively resistant to gemcitabine.

### Example 17

### Expression of the 16D10 Epitope on the Surface of Tumor Cells Treated with Gemcitabine or Cisplatin

Antibody 16D10 binding to SOJ-6 cells following treatment with chemotherapeutic agents was tested in order to assess whether gemcitabine or cisplatin affect the expression of the 16D10 epitope on the BSDL/FAPP polypeptide. Antibody 16D10 binding was assessed by immunofluorescence and flow cytometry following treatment of SOJ-6 cells with 20 μM cisplatin or 5 μM gemcitabine.

For immunofluorescence, SOJ-6 cells were cultured on glass slides and treated or not with gemcitabine or cisplatin, fixed and incubated in the presence of 16D10 and a secondary antibody coupled to fluorescein. Slides were assessed with fluorescence microscope. Results showed staining with 16D10 on slides treated or not with gemcitabine or cisplatin; cells treated with gemcitabine or cisplatin additionally showed clear morphological changes.

Flow cytometry experiments were consistent with immunofluorescence results, as cells treated with each of the drugs continued to be bound on their surface by 16D10. Cisplatin-treated cells however showed a decrease in expression of the 16D10 epitope compared to gemcitabine-treated cells. Results are shown in Table 2.

TABLE 2

| | | 16D10 epitope |
|---|---|---|
| Treatment | Average | expression |
| Control | 418.7 +/− 50.5 | 0 |
| Cisplatin | 324.6 +/− 26.3 | −32.5 |
| Gemcitabine | 419.0 +/− 36.2 | 0 |

The results of the study support treating pancreatic cancer with one or both of cisplatin and gemcitabine together with antibodies that bind the 16D10 epitope as tumor cells do not lose 16D10 epitope expression upon treatment with the chemotherapeutic agent. The study also supports treating pancreatic cancer which has previously been treated with cisplatin and/or gemcitabine, since these cells continue to be target-able by an anti-16D10 epitope antibody.

### Example 18

### Combined Effect of Cisplatin or Gemcitabine with Antibody 16D10 on Human Pancreatic Tumor Cells

SOJ-6 and PANC-1 cells were treated first with gemcitabine or cisplatin, followed by 16D10, and then in the reverse order. In each case proliferation was assessed by MTT assay after 24 hours, expressed as % of control, using increasing concentrations of gemcitabine (5 μM) or cisplatin (20

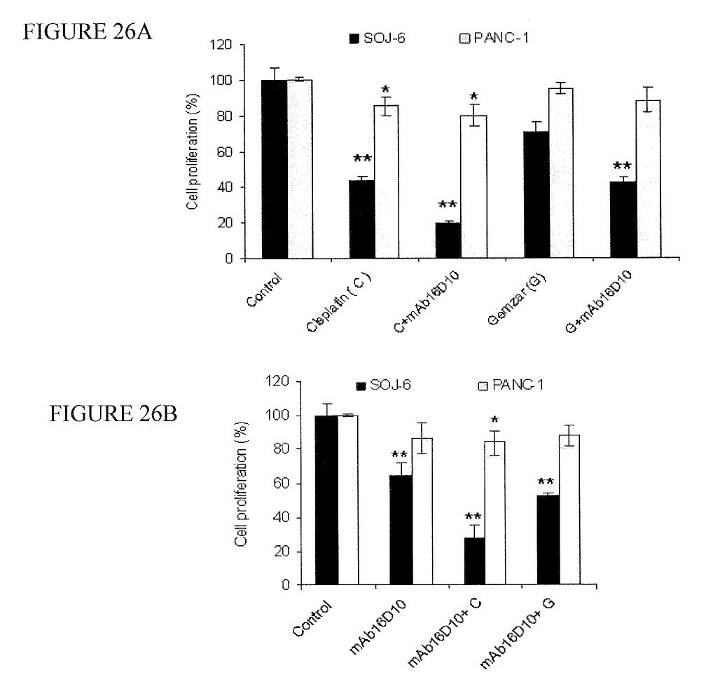

Results for pretreatment with gemcitabine or cisplatin for two hours followed by 16D10 (25 μg/ml) are shown in FIG. 26A. Results for pretreatment with 16D10 (25 μg/ml) for two hours followed by gemcitabine or cisplatin are shown in FIG. 26B. The drug-antibody combination amplifies the anti-proliferative effect by about 25% on SOJ-6, irrespective of the order of treatment. PANC-1 cells used as control shows that these cells remain resistant to all treatments.

### Example 19

### The Combination of Cisplatin or Gemcitabine with Antibody 16D10 Amplifies Apoptosis

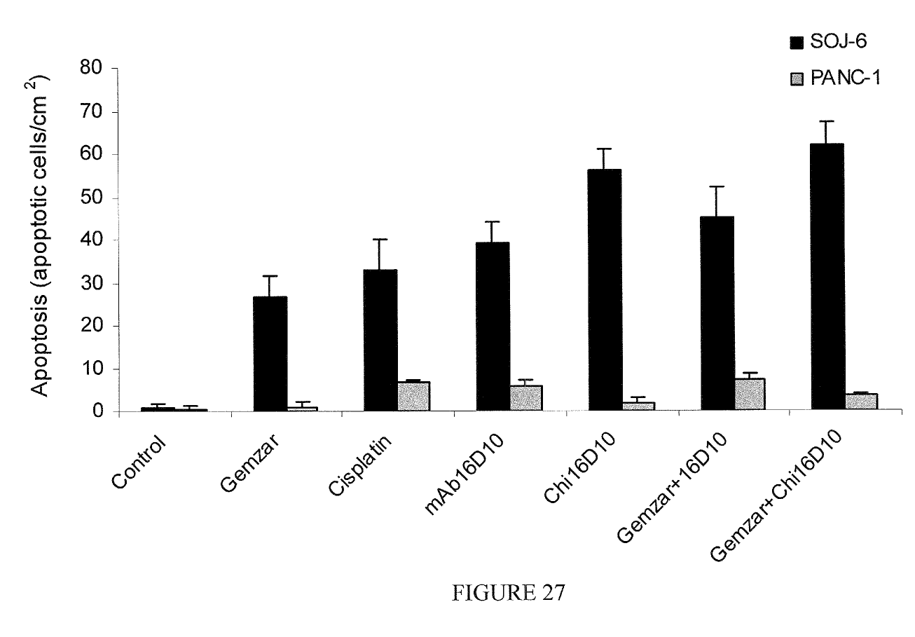

Apoptotic activity was assessed by monitoring caspase activity. SOJ-6 and PANC-1 cells were incubated with gemcitabine and/or antibody 16D10 (25 μg/ml) or recombinant chimeric 16D10 (5 μg/ml). After 24 hours of incubation, caspase activity was measured. Results are shown in FIG. 27, expressed as number of apoptotic cells compared to control (untreated cells). The results showed that gemcitabine alone or with antibody 16D10 or in combination with recombinant chimeric 16D10 induce apoptosis on SOJ-6 cells.

In order to confirm these results by biochemical methods, SOJ-6 were next incubated for 24 hours with gemcitabine or cisplatin and/or antibody 16D10 (25 μg/ml) or recombinant chimeric 16D10 (5 μg/ml) and lysed. Proteins from the cellular lysate were separated by PAGE and transferred to nitrocellulose membrane. Immunodetection was carried out by chemoluminesence in the presence of anti-caspase 9, anti-cleaved caspase 9, anti-PARP and anti-cleaved PARP. Beta-actin served as control. Results showed that caspase 9 as well as PARP was activated in cells treated with gemcitabine and cisplatin alone or in combination with antibody 16D10 or recombinant chimeric 16D10, and that the combination of gemcitabine and cisplatin and 16D10 antibodies amplifies apoptotic by the mitochondrial pathway.

### Example 20

### Activation of Bcl-2 Family Members (Bax and Bcl-2)

In order to assess whether apoptosis of drug combinations is regulated by Bcl-2 family proteins, SOJ-6 cells were incubated for 24 hours with a combination of gemcitabine and cisplatin and 16D10 antibodies, and Bcl-2 family proteins were assessed in proteins from cellular lysate using anti-Bcl-2 and anti-Bax antibodies. An accumulation of Bax together with inhibition of Bcl-2 expression was observed in SOJ-6 cells treated with gemcitabine and cisplatin and 16D10 antibodies, compared to control untreated SOJ-6 cells. The results therefore support activation of the mitochondrial apoptotic pathway by the drug-antibody combination treatment.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

We claim:

1. A method of producing an antigen-binding compound suitable for use in the treatment of cancer, said method comprising:
i) providing an antigen-binding compound that specifically binds to a BSDL or FAPP polypeptide;
ii) testing said antigen-binding compound for pro-apoptotic activity on BSDL- or FAPP-expressing cells;
iii) selecting said antigen-binding compound If it is determined that it has pro-apoptotic activity on BSDL- or FAPP-expressing cells; and
iv) producing a quantity of the selected antigen-binding compound.

2. The method of claim 1, further comprising a step which comprises testing said antigen-binding compound for the ability to induce immune cell mediated killing of BSDL- or FAPP-expressing cells, and selecting said antigen-binding compound If it is determined that it has the ability to induce immune cell mediated killing of BSDL- or FAPP-expressing cells.

3. The method of claim 1, further comprising a step in which said antigen-binding compound is prepared for administration to a human.

4. The method of claim 3, wherein the preparation for administration to a human comprises formulating said compound with a pharmaceutically acceptable carrier.

5. The method of claim 1, wherein said BSDL- or FAPP-expressing cells are tumor cells.

6. The method of claim 1, wherein step is carried out in the absence of immune effector cells.

7. The method of claim 1, wherein step iv) comprises culturing a host cell producing said antigen-binding compound in a suitable medium and recovering said antigen-binding compound.

8. The method of claim 1, wherein said antigen-binding compound is an antibody that specifically binds a BSDL or FAPP polypeptide.

9. The method of claim 1, wherein said antigen-binding compound competes for binding with antibody 16D10 to a BSDL or FAPP polypeptide.

10. The method of claim 9, wherein said antibody has a heavy chain constant region of an IgG isotype.

11. The method of claim 10, wherein said IgG isotype is a human IgG1 isotype.

12. The method of claim 11, wherein said antibody is a chimeric, human or humanized antibody.

13. An antigen-binding compound produced according to the method of claim 1.

14. A pharmaceutical composition comprising the antigen-binding compound of claim 13, and a pharmaceutically acceptable carrier.

15. A bivalent antibody comprised of two heavy chains and two light chains, wherein the heavy chains comprise an IgG heavy chain constant region capable of binding to an Fc receptor, and wherein the antibody:
(a) is capable of inducing apoptosis or inhibiting the proliferation of cells expressing a BSDL or FAPP polypeptide;
(b) is capable of inducing cell-mediated killing (ADCC) of BSDL- or FAPP-expressing cells; and
(c) competes for binding with antibody 16D10 to a BSDL or FAPP polypeptide.

16. A bivalent antibody comprising: (a) a heavy chain comprising a variable region comprising one or more CDRs derived from the amino acid sequence of SEQ ID NO: 7 fused to a human IgG chain constant region; and (b) a light chain comprising a variable region comprising one or more CDRs derived from the amino acid sequence of SEQ ID NO: 8, optionally fused to human kappa chain constant region.

17. The antibody of claim 15, wherein the heavy chain comprises CDR1, CDR2 and CDR3 derived from the amino acid sequence of SEQ ID NO: 7, and the light chain comprises CDR1, CDR2 and CDR3 derived from the amino acid sequence of SEQ ID NO: 8.

18. The antibody of claim 17, wherein the heavy chain comprises the amino acid sequence of SEQ ID NO: 7.

19. The antibody of claim 17, wherein the light chain comprises the amino acid sequence of SEQ ID NO: 8.

20. The antibody of claim 16, wherein the heavy chain comprises CDR1, CDR2 and CDR3 derived from the amino acid sequence of SEQ ID NO: 7, and the light chain comprises CDR1, CDR2 and CDR3 derived from the amino acid sequence of SEQ ID NO: 8.

21. The antibody of claim 20, wherein the heavy chain comprises the amino acid sequence of SEQ ID NO: 7.

22. The antibody of claim 20, wherein the light chain comprises the amino acid sequence of SEQ ID NO: 8.

23. The antibody of claim 15, wherein said heavy chain constant region is a human IgG1.

24. The antibody of claim 23, wherein said antibody is hypofucosylated.

25. The antibody of claim 16, wherein said heavy chain constant region is a human IgG1.

26. The antibody of claim 25, wherein said antibody is hypofucosylated.

27. The antibody of claim 15, wherein said antibody does not comprise a cytotoxic agent selected from the group consisting of a radioactive isotope, a toxic polypeptide, and a toxic small molecule.

28. The antibody of claim 15, wherein said antibody comprises a cytotoxic agent selected from the group consisting of a radioactive isotope, a toxic polypeptide, and a toxic small molecule.

29. The antibody of claim 16, wherein said antibody does not comprise a cytotoxic agent selected from the group consisting of a radioactive isotope, a toxic polypeptide, and a toxic small molecule.

30. The antibody of claim 16, wherein said antibody comprises a cytotoxic agent selected from the group consisting of a radioactive isotope, a toxic polypeptide, and a toxic small molecule.

31. The antibody of claim 15, wherein said antibody is a chimeric, human or humanized antibody.

32. The antibody of claim 16, wherein said antibody is a chimeric, human or humanized antibody.

33. A method of inducing apoptosis of a cell which expresses a BSDL or FAPP polypeptide, comprising exposing the cell to an antigen-binding compound of claim 15 in an amount effective to induce apoptosis of the cell.

34. The method of claim 33, wherein the antigen-binding compound is administered to a subject having pancreatic cancer.

35. The method of claim 34, wherein the subject has an established tumor.

36. The method of claim 35, wherein the antigen-binding compound is administered to a subject in combination with a chemotherapeutic agent.

37. The method of claim 36, wherein said chemotherapeutic agent is selected from the group consisting of: an alkylating agent, an antimetabolite, a cytotoxic antibiotic, a vinca alkaloid, a tyrosine kinase inhibitor, a metalloproteinase inhibitor and a COX-2 inhibitor.

38. The method of claim 36, wherein said chemotherapeutic agent is gemcitabine.
